(19) United States
(12) Patent Application Publication
(10) Pub. No.: US 2009/0029423 A1
(43) Pub. Date: Jan. 29, 2009

(54) METHOD FOR SEPARATING TARGET COMPONENT

(75) Inventors: Takeshi Imamura, Chigasaki-shi (JP); Tetsuya Yano, Atsugi-shi (JP); Tsutomu Honma, Atsugi-shi (JP); Shinya Kozaki, Tokyo (JP); Tsuyoshi Nomoto, Tokyo (JP); Akiko Tsuchitani, Yamato-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/201,338

(22) Filed: Aug. 29, 2008

Related U.S. Application Data
(62) Division of application No. 10/544,942, filed on Aug. 9, 2005.

(30) Foreign Application Priority Data
May 2, 2003 (JP) 2003-127363
May 2, 2003 (JP) 2003-127508
May 2, 2003 (JP) 2003-127592

(51) Int. Cl. C12P 19/34; C12P 7/42

(52) U.S. Cl. 435/91.5; 435/146

(57) ABSTRACT

A construct in which at least a part of the magnetic material is coated with a polyhydroxyalkanoate (PHA), and a method for producing a construct by immobilizing a PHA synthesizing enzyme on the surface of the magnetic material, thereby biosynthesizing and coating a PHA.

This application is a division of application Ser. No. 10/544,942, which is a national stage of International Application No. PCT/JP2004/006420, filed May 6, 2004, and which is incorporated herein by reference.

### TECHNICAL FIELD

The present invention relates to a construct characterized by including a polyhydroxyalkanoate and a magnetic member and by having a structure such that the polyhydroxyalkanoate covers at least a part of the magnetic member, and a producing method therefor.

The present invention also relates to a method for separating a specified target component contained in a specimen, a method for detecting such a target component, and a method for screening a specified component utilizing the construct. More specifically, the present invention relates to a method for a selective separation, detection or screening of a specified target component contained in a specimen by forming a construct bearing, on a carrier surface, a molecule having a specific affinitive coupling property to a specified target component, for example, a nucleic acid molecule, a protein, a peptide, a sugar, a lipid, a low-molecular weight compound or a composite thereof of natural origin or artificially modified nature, and causing such a construct to bond with the specified target component. The present invention also relates to an apparatus exclusively utilizable for executing the afore-described methods.

### BACKGROUND ART

Polymer materials are indispensable for modern industries and lives, and, owing to their features such as inexpensiveness, light weight and satisfactory molding properties, are utilized in various fields, such as a casing of an electric appliance, a packaging material, a cushioning material, a fiber material, etc. Further, utilizing the stability of such polymer materials, various functional materials, such as a liquid crystal material or a coating material, are obtained by introducing substituents capable of exhibiting various functions into the molecular chain of a polymer material. Such a functional material, having a higher added value than in the polymer itself as the structural material, can expect a large market demand even with a small-scale production. Such a functional polymer material has been obtained by methods based on organic synthetic chemistry, in a polymer synthesis process or by a modification of a synthesized polymer with a substituent. The polymer, constituting the basic skeletal structure of the functional polymer material, is obtained, in most cases, from petroleum-based raw materials by methods based on organic synthetic chemistry. Typical examples of such a polymer include polyethylene, polyethylene terephthalate, polyester, polystyrene, polyvinyl chloride and polyacrylamide.

Multi-Layered Construct Containing Magnetic Member

The present inventors have investigated a multi-layered construct in which a magnetic material is coated by a polymer compound, as an elementary technology for providing a polymer compound with a high added value. Such coating of a specified magnetic material with a polymer compound can provide a composite construct having an extremely useful functionality. Applications of such a construct include, for example, a carrier being a microcapsule construct containing a magnetic material in a polymer compound for separating, purifying or screening a biological substance, and a magnetic recording medium formed by coating a sheet-shaped magnetic material with a polymer compound.

Since such a microcapsule construct containing magnetic material can be easily collected by a magnetic force, and excellent effects can be expected principally in biochemical fields if it is used, for example, as a carrier for a medical diagnostic drug, a carrier for separating germs or cells, a carrier for separating and purifying nucleic acid or protein, a carrier for drug delivery, a carrier for an enzyme reaction, or a carrier for a cell culture. The capsule construct containing a magnetic material can be synthesized, for example, by a method of dispersing an oleophilized magnetic material in a polymerizable monomer and executing suspension polymerization (Japanese Patent Application Laid-Open No. S59-221302), a method of dispersing an oleophilized magnetic material in a polymerizable monomer, and executing polymerization under homogenization in water with a homogenizer, thereby obtaining magnetic particles of a relative small particle size (Japanese Patent Publication No. H04-3088), or a method of precipitating and oxidizing an iron compound in the presence of porous polymer particles having a specified functional group to introduce a magnetic material into the interior of the porous polymer particles, thereby obtaining magnetic particles of a large and uniform particle size (Japanese Patent Publication No. H05-10808).

However, in the case where such a magnetic material-containing capsule construct, obtained by these synthesizing methods, is applied, for example, to the carrier for the medical diagnostic drug, a practically acceptable performance is often not obtained because of a significant loss in the sensitivity or by a non-specific reaction even when most of the magnetic material is present inside the capsule construct. This is presumably because the magnetic material is partially exposed on the surface of the capsule construct or a micropath is formed between the surface of the construct and the internal magnetic material, whereby the magnetic component is dissolved out to hinder the practical performance. The magnetic material generally is more hydrophilic than the polymer particles, and, in the conventional synthesizing methods, the magnetic material tends to be localized at the surface of the capsule construct or in the vicinity thereof, and such a fact constitutes a major reason for deterioration of the practical performance. As explained above, in the conventional magnetic material-containing capsule construct, it is difficult to suppress the elution of the magnetic component due to the exposure of the magnetic material on the surface or the micropath formation, and the application of such a capsule construct is in fact limited to fields where such elution is not problematic.

Also, various attempts have been reported to improve the surface characteristics of the magnetic material, thereby improving dispersibility of the magnetic material in a polymerized toner. Japanese Patent Application Laid-Open Nos. S59-200254, S59-200256, S59-200257 and S59-224102 propose treating a magnetic material with various silane coupling agents. Also, Japanese Patent Application Laid-Open Nos. S63-250660 and H10-239897 disclose a technology for treating silicon-containing magnetic particles with a silane coupling agent.

However, though these technologies can improve the dispersibility of the magnetic particles to a certain extent, it is difficult to attain uniform hydrophobicity of the surface of the magnetic material, and a further improvement has been desired in order to prevent mutual uniting of the magnetic particles and generation of non-hydrophobic magnetic particles and to improve the dispersibility of the magnetic material to a satisfactory level.

Also, as an example of utilizing a hydrophobicized magnetic iron oxide, Japanese Patent Publication No. S60-3181 proposes a toner containing magnetic iron oxide treated with an alkyltrialkoxy silane. Though the addition of such a magnetic iron oxide provides a certain improvement in the electrophotographic properties of the toner, further improvement is still desirable because the originally low surface activity of magnetic iron oxide tends to cause fused particles or uneven hydrophobicity in the course of treatment.

PHA

Meanwhile, research for producing a polymer compound by a biological method has been actively carried out in recent years and the results are being commercialized in part. Examples of polymer compounds derived from microorganisms include a polyhydroxyalkanoate, such as poly-3-hydroxy-n-butyric acid (hereinafter also abbreviated as PHB) or a copolymer of 3-hydroxy-n-butyric acid and 3-hydroxy-n-valeric acid (hereinafter also abbreviated as PHB/V), a polysaccharide, such as bacterial cellulose or purlan, a polyamino acid, such as poly-γ-glutamic acid and polylysine. A polyhydroxyalkanoate (hereinafter also referred to as PHA) means a polyhydroxyalkanoate containing a hydroxy alkanoic acid unit. In particular, a PHA can be utilized in various products, for example, by melt-forming, like conventional plastics. Also, it shows satisfactory biocompatibility and is expected as a soft material for medical use.

It has been reported that many microorganisms produce PHA and accumulate it within cells. For example, microbial production of PHB/V by Alcaligenes eutrophus H16 (ATCC No. 17699), Methylobacterium sp., Paracoccus sp., Alcaligenes sp., and Pseudomonas sp. has been reported (for example, Japanese Patent Application Laid-Open No. H05-7492, Japanese Patent Publication Nos. H06-15604, H07-14352, and H08-19227). Furthermore, Comamonas acidovorans IFO 13852 produces PHA comprised of monomer units of 3-hydroxy-n-butyric acid and 4-hydroxy-n-butyric acid (Japanese Patent Application Laid-Open No. H09-191893), and Aeromonas caviae produces a copolymer of 3-hydroxy-n-butyric acid and 3-hydroxyhexanoic acid (Japanese Patent Application Laid-Open Nos. H05-93049 and H07-265065).

A polyhydroxyalkanoate constituted of a 3-hydroxyalkanoic acid unit of a short-chain-length, such as PHB or PHB/V (such PHA is hereinafter also abbreviated as scl-PHA), is synthesized by an enzymatic polymerization reaction using as a substrate at least one of (R)-3-hydroxybutyryl CoA, (R)-3-hydropropionyl CoA and (R)-3-hydroxyvaleryl CoA, which are synthesized from various carbon sources through various in vivo metabolic pathways. The enzyme that catalyzes this polymerization reaction is called scl-PHA synthetase in the present invention. For example, an enzyme synthesizing PHB is called PHB synthetase (also called PHB polymerase or PHB synthase). CoA is an abbreviation for coenzyme A, and its chemical structure is represented by the following chemical formula:

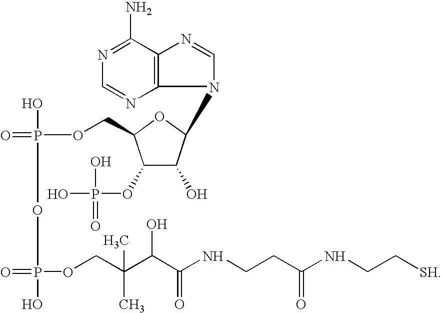

Recently, active studies on polyhydroxy alkanoate comprised of 3-hydroxyalkanoic acid units of medium-chain-length (about 3 to 12 carbon atoms) (mcl-PHA) have been conducted.

For example, Japanese Patent No. 2642937 discloses that Pseudomonas oleovorans ATCC 29347 can produce PHA comprised of 3-hydroxyalkanoic acid monomer units of 6 to 12 carbon atoms from non-cyclic aliphatic hydrocarbons. In addition, it has been reported, in Appl. Environ. Microbiol., 58, 746 (1992), that Pseudomonas resinovorans produces PHA of which monomer units are 3-hydroxy-n-butyric acid, 3-hydroxyhexanoic acid, 3-hydroxyoctanoic acid, and 3-hydroxydecanoic acid using octanoic acid as a sole carbon source, and it also produces PHA of which monomer units are 3-hydroxy-n-butyric acid, 3-hydroxyhexanoic acid, 3-hydroxyoctanoic acid, and 3-hydroxydecanoic acid using hexanoic acid as sole carbon source. Here, the 3-hydroxyalkanoic acid monomer units longer than the raw material fatty acid are considered derived from the fatty acid synthesizing pathway described below.

Int. J. Biol. Macromol., 16 (3), 119 (1994) reported that Pseudomonas sp. Strain 61-3 produces PHA comprised of monomer units of 3-hydroxyalkanoic acids, such as 3-hydroxy-n-butyric acid, 3-hydroxyhexanoic acid, 3-hydroxyoctanoic acid, and 3-hydroxydecanoic acid, and 3-hydroxyalkanoic acids such as 3-hydroxy-5-cis-decenoic acid and 3-hydroxy-5-cis-dodecenoic acid, using sodium gluconate as a sole carbon source.

The above-described PHAs are PHAs comprised of monomer units having alkyl groups as the side chain (hereinafter also called usual-PHAs), or analogs thereof (for example, including an alkenyl group having a double bond on a side chain other than at the end portion). However, when a wider application of a PHA, e.g., as a functional polymer, is intended, a PHA having side chains including a substituent other than an alkyl group (for example, a phenyl group, an unsaturated hydrocarbon, an ester group, an allyl group, a cyano group, a halogenated hydrocarbon, or an epoxide) is extremely useful (unusual-PHA).

As for biosynthesis of unusual-PHA having a phenyl group, it is reported that Pseudomonas oleovorans produces PHA having 3-hydroxy-5-phenylvaleric acid units from 5-phenylvaleric acid (Macromolecules, 24, 5256-5260 (1991), Macromol. Chem., 191, 1957-1965 (1990), Chirality, 3, 492-494 (1991)). Also, Macromolecules, 29, 1762-1766 (1996) reports that Pseudomonas oleovorans produces PHA having 3-hydroxy-5-(4-tolyl)valeric acid units from 5-(4-tolyl)valeric acid (5-(4-methylphenyl)valeric acid). Further, Macromolecules, 32, 2889-2895 (1999) reports that Pseudomonas oleovorans produces PHA having 3-hydroxy-5-(2,4-dinitrophenyl)valeric acid units and 3-hydroxy-5-(4-nitrophenyl)valeric acid units from 5-(2,4-dinitrophenyl)valeric acid.

As for unusual-PHA having a phenoxy group, Macromol. Chem. Phys., 195, 1665-1672 (1994) reports that Pseudomonas oleovorans produces PHA having a 3-hydroxy-5-phenoxyvaleric acid unit and a 3-hydroxy-9-phenoxynonanoic acid unit from 11-phenoxyundecanoic acid. Also, Macromolecules, 29, 3432-3435 (1996) reports that Pseudomonas oleovorans produces a PHA having a 3-hydroxy-4-phenoxybutyric acid unit and a 3-hydroxy-6-phenoxyhexanoic acid unit from 6-phenoxyhexanoic acid, a PHA having a 3-hydroxy-4-phenoxybutyric acid unit, a 3-hydroxy-6-phenoxyhexanoic acid unit, and a 3-hydroxy-8-phenoxyoctanoic acid unit from 8-phenoxyoctanoic acid, and a PHA having a 3-hydroxy-5-phenoxyvaleric acid unit and a 3-hydroxy-7-phenoxyheptanoic acid unit from 11-phenoxyundecanoic acid. Further, Can. J. Microbiol., 41, 32-43 (1995) reports that Pseudomonas oleovorans ATCC 29347 and Pseudomonas putida KT 2442 produce PHA having a 3-hydroxy-p-cyanophenoxyhexanoic acid unit and PHA having a 3-hydroxy-p-nitrophenoxyhexanoic acid unit from p-cyanophenoxyhexanoic acid and p-nitrophenoxyhexanoic acid respectively. Japanese Patent No. 2989175 describes a homopolymer comprised of a 3-hydroxy-5-(monofluorophenoxy)valeric acid unit or a 3-hydroxy-5-(difluorophenoxy)valeric acid unit and a copolymer containing at least a 3-hydroxy-5-(monofluorophenoxy)pentanoate unit or a 3-hydroxy-5-(difluorophenoxy)pentanoate unit and a method for producing such homopolymer or copolymer, reciting that such homopolymer and copolymer can provide water-repellency and stereoregularity maintaining high melting point and good workability.

As an example of unusual-PHA having a cyclohexyl group, Macromolecules, 30, 1611-1615 (1997) reports that Pseudomonas oleovorans produces such a PHA from cyclohexylbutyric acid or cyclohexylvaleric acid.

These mcl-PHA and unusual-PHA are synthesized through an enzymatic polymerization reaction using (R)-3-hydroxyacyl CoA as a substrate. (R)-3-hydroxyacyl CoA is generated through metabolic pathways, such as a β-oxidationpathway. The enzyme that catalyzes this polymerization reaction is called, in the present invention, a PHA synthetase. In the present invention, the afore-described scl-PHA synthetase and the mcl-synthetase are collectively called a PHA synthetase, but the mcl-PHA synthetase is often called a PHA synthetase (also called PHA polymerase or PHA synthase). The following is the reaction route from an alkanoic acid to a PHA via the β-oxidation pathway and a polymerization reaction by the PHA synthetase.

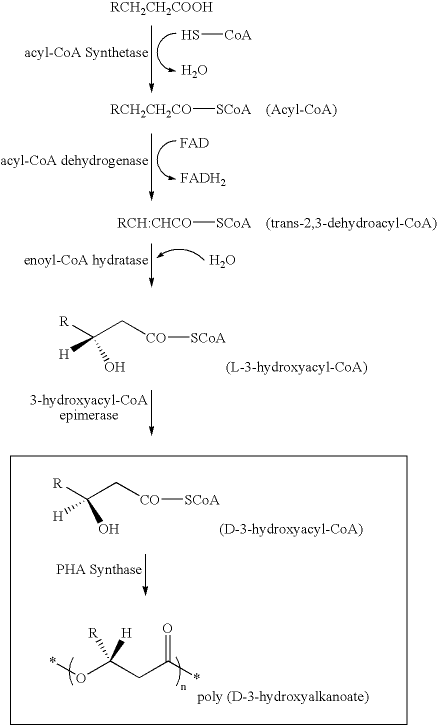

On the other hand, when the production is performed through the fatty acid synthesis pathway, it is considered that (R)-3-hydroxyacyl-ACP (ACP means acyl carrier protein) generated in this pathway is converted to (R)-3-hydroxyacyl CoA from which PHA is synthesized by the PHA synthetase.

In Vitro PHA Synthesis Utilizing Enzyme

Recently, attempts have been made to synthesize PHA in vitro using a PHA synthetase (scl-PHA synthetase or mcl-PHA synthetase) isolated from cells.

For example, Proc. Natl. Acad. Sci. USA, 92, 6279-6283 (1995) describes that PHB comprised of 3-hydroxy-n-butyric acid units is successfully synthesized from 3-hydroxybutyryl CoA by the action of scl-PHA synthetase derived from Alcaligenes eutrophus. In addition, Int. J. Biol. Macromol., 25, 55-60 (1999) describes that PHA comprised of a 3-hydroxy-n-butyric acid unit or a 3-hydroxy-n-valeric acid unit is successfully synthesized from 3-hydroxybutyryl CoA or 3-hydroxyvaleryl CoA using scl-PHA synthetase derived from Alcaligenes eutrophus. Further, this report mentions that, when racemic 3-hydroxybutyryl CoA is reacted, PHB comprised of only (R)-3-hydroxy-n-butyric acid units is successfully synthesized due to the stereoselectivity of the enzyme. Macromol. Rapid Commun., 21, 77-84 (2000) reports in vitro PHB synthesis using scl-PHA synthetase derived from Alcaligenes eutrophus.

FEMS Microbiol. Lett., 168, 319-324 (1998) describes that PHB comprised of a 3-hydroxy-n-burytic acid unit is successfully synthesized by reacting PHB synthetase derived from Chromatium vinosum on 3-hydroxybutyryl CoA.

In Appl. Microbiol. Biotechnol., 54, 37-43 (2000), PHA comprised of 3-hydroxydecanoic acid is synthesized by reacting PHA synthetase derived from Pseudomonas aeruginosa on 3-hydroxydecanoyl CoA.

In addition to the foregoing, the present invention cites descriptions of Japanese Patent No. 2989175, Japanese Patent Application Laid-Open Nos. 2001-78753 and 2001-69968, Eur. J. Biochem., 250, 432-439 (1997), J. Biol. Chem., 218, 97-106 (1956), J. Amer. Chem. Soc., 78, 2278 (1956), Appl. Environ. Microbiol., 44, 238-241 (1982), Molecular Cloning, vol. 1, 572, 1989 (Cold Spring Harbor Laboratory), J. Bacteriol., 182, 2753-2760 (2000), and Int. J. Biol. Macromol., 12, 85-91 (1990).

Meanwhile, for separation/recovery, detection and screening of a target component contained in a specimen, particularly a target component effective for medical treatment or diagnosis or useful industrially, there are being developed and utilized various methods of separation/recovery, detection and screening, utilizing fine particles from a micrometer to a nanometer in size as a carrier for a molecule having a coupling affinity to the target component, such as a probe molecule. In particular, a method utilizing fine particles having a magnetic property (hereinafter called magnetic particles) as the afore-described carrier has an advantage in that, at the separation or recovery of the carrier from the specimen, the magnetic particles can be easily separated or recovered by a magnetic force. For this reason, many developments have been made in the method utilizing the magnetic particles.

The magnetic particles utilized as the carrier for immobilizing the afore-described probe molecule or the like are mostly used in a state in which the surface is coated with an organic polymer to improve its stability and control its magnetic properties.

As an application of the magnetic particles coated with the organic polymer as such a carrier, immunoassay utilizing an antigen-antibody reaction has been developed.

As an example, Japanese Patent Application Laid-Open No. H07-151755 discloses an immunoassay utilizing magnetic material-containing polystyrene latex of an average particle size of 0.7 μm, manufactured by Rhone-Poulenc.

Also, Japanese Patent Application Laid-Open No. H10-221341 discloses an immunological measuring method utilizing tocylated magnetic particles (Dynabead M-280, average particle size 2.8 μm), manufactured by Nippon Dynal Co. Also, Japanese Patent Application Laid-Open No. H09-229936 discloses an immunological assay method and an apparatus, utilizing magnetic particles Dynabeads M-450 uncoated, manufactured by Dynal Inc., of a particles size of 4.5 μm, 3% (w/v).

Another application area of the magnetic particles coated with the organic polymer as a carrier is an inspection/diagnosis method for nucleic acid molecules, such as DNA.

Japanese Patent Application Laid-Open No. H05-281230 discloses an antigen-antibody reaction and an inspection/diagnosis method for nucleic acid molecules, such as DNA, utilizing, as magnetic carrier particles, XP-600 manufactured by Dino Industrier A.S., Norway.

In addition, as still another application area of such magnetic particles, there are developed methods for separating and recovering a target component.

Japanese Patent Application Laid-Open No. H09-304385 discloses a method and an apparatus for separation and recovery of basophilic cells, utilizing Dynabeads M-450 uncoated (Dynal Inc., particle size of magnetic particles: 45 μm).

Japanese Patent Application Laid-Open No. H10-068731 discloses a method for magnetically separating an object component in liquid, utilizing magnetic particles manufactured by Rhone-Poulenc, as magnetic particles to which an immunologically active substance or nucleic acid is covalently bonded.

Also U.S. Pat. Nos. 4,230,685, 3,970,518, 5,508,164, 5,567,326 and 4,018,886 disclose methods for using magnetic particles to bind a target component thereto and separating the target component bonded with the magnetic particles.

U.S. Pat. No. 5,900,481 discloses a method of binding DNA using coated magnetic particles to treat the DNA.

U.S. Pat. No. 5,834,197 discloses a method of capturing a certain bacterial strain from liquid, utilizing coated magnetic particles, where a labeled antibody having a selective affinity to an antibody is attached to the beads, thereby coupling the detectable label to the magnetic particles, in order to achieve easy detection and recovery of the antigen reacted with the labeled antibody.

In addition to the afore-described patent references, there are references relating to the manipulation of various molecules to be bonded to the magnetic particles, such as Analytical Chemistry, 68(13), 2122-26 (1996) and Nucleic Acids Research 23(16), 3126-31 (1995).

Also, in addition to the magnetic particles described in the foregoing, there are magnetic particles already commercialized for use in methods of detecting, recovering or screening a target component, such as Ferromagnetic Particles from Spherotech Inc., Cera-Mag from Seradyn Inc. and Esteapor from Bangs Laboratory Inc.

### DISCLOSURE OF THE INVENTION

As explained above, an application of a bioengineering method to the synthesis of a polymer compound is expected to enable synthesis of a novel polymer compound or endowment of novel function or structure that have been difficult to realize in the conventional organosynthetic methods. Also, a biological process may often be a one step process, where conventional organosynthetic methods require multiple steps, and there are expected process simplification, cost reduction, time reduction, etc. It is also rendered possible to reduce the amounts of organic solvent, acid, alkali, surfactant, etc., to employ milder reaction conditions and to achieve synthesis from a non-petroleum raw material or a low-purity raw material, thereby realizing a synthesizing process that leads to a lower environmental impact and recycling. With respect to the synthesis from a low-purity raw material, the bioengineering synthetic process can carry out the desired reaction even with a raw material of a low purity because the enzyme, functioning as a catalyst, generally has a high substrate specificity, so that the utilization of a wasted material or a recycled raw material can also be expected.

On the other hand, as explained in the foregoing, the present inventors have investigated a construct in which a magnetic material is coated with a polymer compound, as an elementary technology for providing a polymer compound of a high added value. Such coating of a specified magnetic material with a polymer compound can provide a composite construct having an extremely useful functionality. Various attempts have been made to produce such a construct through organosynthetic methods, but such methods have certain limitations.

If such a construct can be prepared by a bioengineering method, it is expected to realize utilization of a novel polymer compound and endowment of novel functions and structures, which has not been realized in the conventional organosynthetic methods, and also to realize a manufacturing process of a lower environmental impact and resource recycling with a lower cost. For example, based on extremely strict molecular recognition and stereospecificity inherent to the biological catalytic action, it is possible to produce a polymer compound of a novel functionality that has been difficult to realize in the conventional organosynthetic methods, or a capsule construct or a multi-layered construct coated with a polymer compound of an extremely high chirality, by an extremely simple process of a low environmental impact.

Therefore, an object of the present invention is to provide a polymer compound construct of a high functionality producible by a bioengineering process. The invention also provides an efficient method for producing a construct formed by coating a magnetic material with a polymer compound and usable in various fields as a functional composite construct.

In particular, the invention provides a construct of a coated magnetic material without an oleophilic treatment on a metal or a metal compound having magneticity and with an excellent uniformity in dispersion, and a manufacturing method therefor.

As explained in the foregoing, the magnetic material-containing capsule construct obtained by conventional synthetic methods has a drawback of elution of metal ions to the exterior and is currently usable only in the fields and applications where the metal ion elution does not matter. The invention provides a magnetic material-containing capsule construct that is excellent in dispersibility and magnetic response, and hardly causes elution of the metal ions to the outside, thereby being widely applicable in various fields and application, and a producing method therefor.

Also in the method for separation/recovery, detection or screening of a target component, since such a target component often has a physiological activity or a medical/diagnostic efficacy, it is desirable to conduct the operations in an environment as close as possible to in vivo conditions, so as not to deteriorate the physiological properties. However, in the afore-described methods employed in the conventional carrier including the magnetic particles, since the coating layer on the surface of the magnetic particles is made of a synthetic polymer, such as styrenic, acrylic or vinylic, there still remain drawbacks of a non-specific adsorption and a loss in the functionality resulting from a leakage of a monomer component remaining in the synthetic polymer. Also, not only the target component, but also a target-binding molecule to be borne and immobilized on the carrier including the magnetic particles are often substances obtained or derived from a living organism, and the coupling ability of such a target-binding molecule with the target component may be detrimentally affected by the non-natural polymer surface employed in the coating layer of the carrier containing the magnetic particles.

The present invention is intended to resolve these drawbacks, and to provide, in executing separation/recovery, detection or screening of a target component utilizing a construct formed by bearing and immobilizing a target-binding molecule on a carrier, a method of executing separation/recovery, detection or screening of the target component while maintaining the target component and the target-binding molecule borne and immobilized on the carrier under conditions close to those experienced in vivo.

Also, the invention relates to a construct in which at least a part of magnetic material is coated with a polyhydroxyalkanoate.

Also, the invention relates to a method for producing a construct formed by a magnetic material of which at least a part is coated with a polyhydroxyalkanoate, the method being characterized by including a step of immobilizing a polyhydroxyalkanoate synthetase on a surface of the magnetic material and a step of polymerizing 3-hydroxyacyl co-enzyme A by such an enzyme, thereby synthesizing a polyhydroxyalkanoate, whereby such a synthesizing step coats at least a part of the magnetic material with the polyhydroxyalkanoate.

Furthermore, the invention relates to a method for separating a target component contained in a specimen, characterized in including a step of preparing a carrier in which a molecule having a coupling affinity to the target component is immobilized on a surface, a step of mixing the carrier and the specimen, a step of coupling the target component contained in the specimen, to be mixed in the mixing step, with the molecule immobilized on the carrier surface and having the coupling affinity, and a step of separating the target component, immobilized to the carrier in the coupling step through the coupling with the molecule having the coupling affinity, together with the carrier from the specimen, wherein the carrier is at least partly coated with a polyhydroxyalkanoate.

Furthermore, the invention relates to a method for detecting a target component contained in a specimen, characterized in including a step of preparing a carrier in which a molecule having a coupling affinity to the target component is immobilized on a surface, a step of mixing the carrier and the specimen, a step of coupling the target component contained in the specimen, to be mixed in the mixing step, with the molecule immobilized on the carrier surface and having the coupling affinity, and a step of selectively detecting the target component, immobilized to the carrier in the coupling step through the coupling with the molecule having the coupling affinity, wherein the carrier is at least partly coated with a polyhydroxyalkanoate.

Furthermore, the invention relates to a method for screening a target component contained in a medium, for a mixed specimen containing a mixture including the target component in the medium, characterized by including a step of preparing a carrier in which a molecule having a coupling affinity to the target component is immobilized on a surface, a step of mixing the carrier and the specimen, a step of coupling the target component contained in the specimen, to be mixed in the mixing step, with the molecule immobilized on the carrier surface and having the coupling affinity, and a step of separating the target component, immobilized to the carrier in the coupling step through the coupling with the molecule having the coupling affinity, together with the carrier from the mixed specimen, wherein the carrier is at least partly coated with a polyhydroxyalkanoate.

### BRIEF DESCRIPTION OF THE DRAWINGS

FIG. 1 is a gas chromatography-mass spectroscopy chart of a methyl esterified substance derived from a 3-hydroxyoctanoic acid unit of PHA, identified in Example 11;

FIG. 2 is a gas chromatography-mass spectroscopy chart of a methyl esterified substance derived from a 3-hydroxy-5-phenylvaleric acid unit of PHA, identified in Example 14;

FIG. 3 is a gas chromatography-mass spectroscopy chart of a methyl esterified substance derived from a 3-hydroxy-5-(4-fluorophenyl)valeric acid unit of PHA, identified in Example 15;

FIG. 4 is a gas chromatography-mass spectroscopy chart of a methyl esterified substance derived from a 3-hydroxyoctanoic acid unit contained in PHA and identified in Example 26;

FIG. 5 is a schematic view showing a configuration of a magnetic capsule construct bearing a target-binding molecule on a PHA being a surface coating of a carrier (magnetic material);

FIG. 6 is a view schematically showing a selective binding-forming process between the target component and the target-binding molecule on the magnetic capsule construct bearing the target-binding molecule; and

FIG. 7 is a view schematically showing a magnetic separation process of the invention for the magnetic capsule construct bearing the target-binding molecule and forming a selective binding with the target component, based on magneticity of the construct.

### BEST MODE FOR CARRYING OUT THE INVENTION

Construct

The construct of the present invention has a configuration in which a magnetic material is coated with PHA including a monomer unit of various structures having a substituent in a side chain, and is extremely useful as a carrier for separating, purifying or screening microorganisms, cells, nucleic acid, protein or other biological substances, a carrier for a medical diagnostic agent enabling displacement control in a living organism, a carrier for drug delivery for carrying a drug to a diseased part of a patient, a carrier for immobilizing an enzyme, or a functional carrier for a magnetic toner, a magnetic ink, a magnetic paint or a magnetic recording medium. Now the present invention will be explained in detail in the following.

### PHA

A polyhydroxyalkanoate usable in the present invention includes a short-chain-length polyhydroxyalkanoate (short-chain-length PHA, hereinafter also referred to as scl-PHA), in which monomer units are constituted of a 3-hydroxyalkanoic acid unit with 4 or 5 carbon atoms; and a medium-chain-length polyhydroxyalkanoate (hereinafter also referred to as mcl-PHA), in which monomer units include not only a polyhydroxybutyrate (PHB) with 4 carbon atoms or polyhydroxyvalerate (PHV) with 5 carbon atoms, but also a 3-hydroxyalkanoic acid unit with about 6 to 12 carbon atoms. In addition to the afore-described polyhydroxyalkanoate constituted of a monomer unit having an alkyl group in the side chain (hereinafter also referred to as usual-PHA), the present invention can utilize a polyhydroxyalkanoate including a monomer unit in which various substituents (such as a phenyl group, an unsaturated hydrocarbon group, an ester group, an aryl group, a cyano group, a halogenated hydrocarbon group, or an epoxy (—O—) group) other than an alkyl group are introduced in the side chain in consideration of applications in wider fields, such as a functional polymer (such PHA being hereinafter represented also as unusual-PHA), or a copolymer including these monomer units in an arbitrary unit ratio.

PHA to be employed in the method of the invention is not particularly restricted as long as it is synthesizable by a PHA synthetase (for example, mcl-PHA and unusual-PHA described above). As explained in the foregoing, the PHA synthetase is an enzyme catalyzing a final step in a PHA synthesizing reaction system in vivo, and any PHA known to be synthesized in vivo is synthesized by the catalytic action of such an enzyme. In the invention, it is therefore possible, by reacting a 3-hydroxyacyl CoA corresponding to a desired PHA with the PHA synthetase immobilized to the magnetic material, to prepare a construct in which the magnetic material coated with any PHA known to be synthesized in vivo.

Specific examples of such PHA include PHA including at least monomer units represented by following chemical formulae [1] to [10] and [A] to [D].

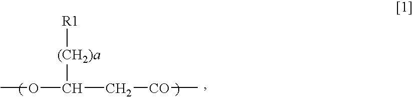

wherein the monomer unit is at least one selected from a group of monomer units having following combinations of R1 and a:

a monomer unit in which R1 is a hydrogen atom (H) and a is any of integers from 3 to 10;

a monomer unit in which R1 is a halogen atom and a is any of integers from 1 to 10;

a monomer unit in which R1 is a chromophore and a is any of integers from 1 to 10;

a monomer unit in which R1 is a carboxyl group or a salt thereof and a is any of integers from 1 to 10; and

a monomer unit in which R1 is

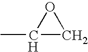

and a is any of integers from 1 to 7.

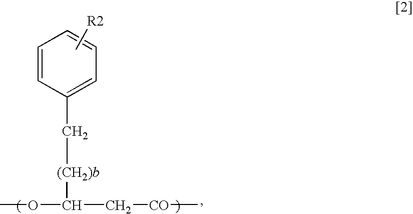

wherein b represents any of integers from 0 to 7; R2 represents any one selected from a group of a hydrogen atom (H), a halogen atom, —CN, —NO₂, —CF₃, —C₂F₅, —C₃F₇, a CH₃ group, a C₂H₅ group, a C₃H₇ group, a vinyl group, an epoxy group and COOR21 (R21 representing an H atom, an Na atom or a K atom); and, in the presence of plural units, the foregoing stands independently for each unit.

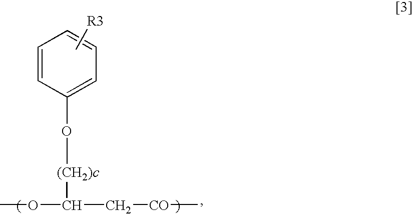

wherein c represents any of integers from 1 to 8 and R3 represents any one selected from a group of a hydrogen atom (H), a halogen atom, —CN, —NO₂, —CF₃,
—C₂F₅, —C₃F₇, a CH₃ group, a C₂H₅ group, a C₃H₇ group and a SCH₃ group; and, in the presence of plural units, the foregoing stands independently for each unit.

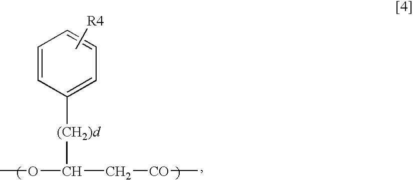

wherein d represents any of integers from 0 to 8; R4 represents any one selected from a group of an H atom, a CN group, a NO₂ group, a halogen atom, a CH₃ group, a C₂H₅ group, a C₃H₇ group, a CF₃ group, a C₂F₅ group, and a C₃F₇ group; and, in the presence of plural units, the foregoing stands independently for each unit.

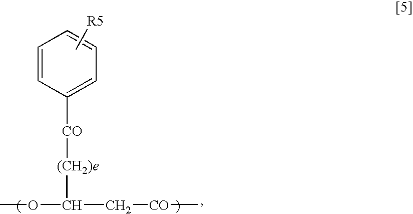

wherein e represents any of integers from 1 to 8 and R5 represents any one selected from a group of a hydrogen atom (H), a halogen atom, —CN, —NO₂, —CF₃, —C₂F₅, —C₃F₇, —CH₃, —C₂H₅, and —C₃H₇.

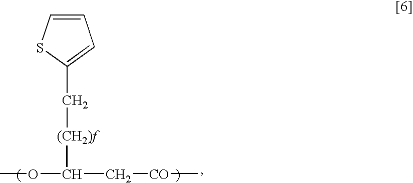

wherein f represents any of integers from 0 to 7.

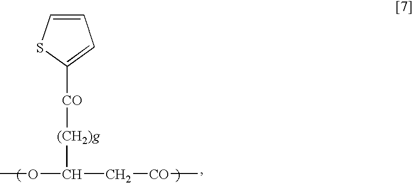

wherein g represents any of integers from 1 to 8.

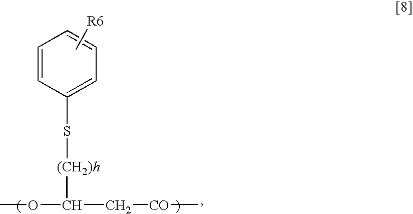

wherein h represents any of integers from 1 to 7; R6 represents any one selected from a group of a hydrogen atom (H), a halogen atom, —CN, —NO₂, —COOR′, —SO₂R″, —CH₃, —C₂H₅, —C₃H₇, —CH(CH₃)₃, and —C(CH₃)₃; R′ represents a hydrogen atom (H), Na, K, —CH₃, or —C₂H₅; and R″ represents —OH, —ONa, —OK, a halogen atom, —OCH₃, or —OC₂H₅.

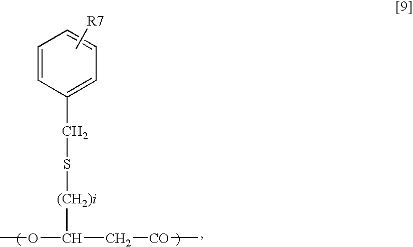

wherein i represents any of integers from 1 to 7; R7 represents any one selected from a group of a hydrogen atom (H), a halogen atom, —CN, —NO₂, —COOR′, and —SO₂R″; R′ represents a hydrogen atom (H), Na, K, —CH₃, or —C₂H₅; and R″ represents —OH, —ONa, —OK, a halogen atom, —OCH₃, or —OC₂H₅).

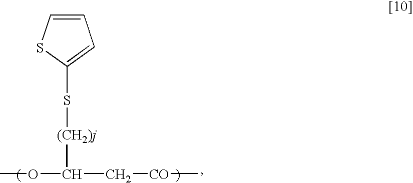

wherein j represents any of integers from 1 to 9.

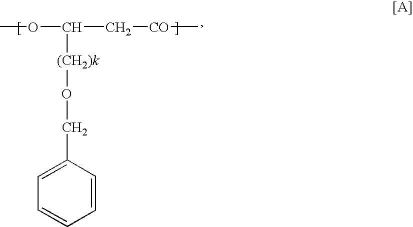

wherein k represents any of integers from 1 to 8.

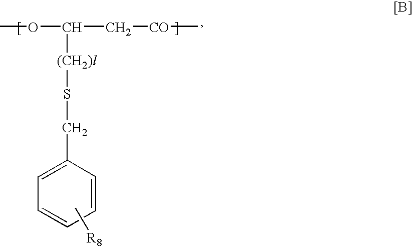

wherein 1 represents any of integers from 1 to 8; R8 represents any one selected from a group of a CH₃ group, a C₂H₅ group, a C₃H₇ group, a (CH₃)₂—CH group and a (CH₃)₃—C group; and, in the presence of plural units, the foregoing stands independently for each unit.

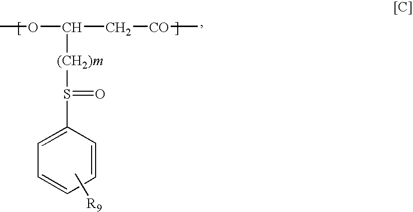

wherein m represents any of integers from 1 to 8; R9 represents an H atom, a halogen atom, a CN group, a NO₂ group, COOR91, SO₂R92 (R91 representing H, Na, K, CH₃ or C₂H₅, and R92 representing OH, ONa, OK, a halogen atom, OCH₃ or OC₂H₅), a CH₃ group, a C₂H₅ group, a C₃H₇ group, a (CH₃)₂—CH group or a (CH₃)₃—C group; and, in the presence of plural units, the foregoing stands independently for each unit.

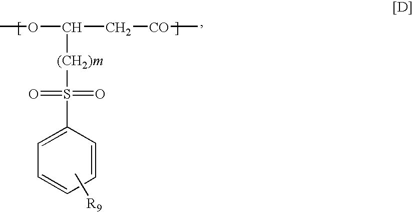

wherein m represents any of integers from 1 to 8; R9 represents an H atom, a halogen atom, a CN group, a NO₂ group, COOR91, SO₂R92 (R91 representing H, Na, K, CH₃ or C₂H₅, and R92 representing OH, ONa, OK, a halogen atom, OCH₃ or OC₂H₅), a CH₃ group, a C₂H₅ group, a C₃H₇ group, a (CH₃)₂—CH group or a (CH₃)₃—C group; and, in the presence of plural units, the foregoing stands independently for each unit.

Specific examples of the afore-described halogen atom include fluorine, chlorine, and bromine. The chromophore is not particularly limited as long as the 3-hydroxyacyl CoA having the chromophore is catalyzed by the PHA synthetase, but it is preferable that a methylene chain of 1 to 5 carbon atoms is present between the chromophore and the carboxyl group to which CoA is bonded, in view of steric hindrance at the time of polymer synthesis. When the light absorption wavelength of the chromophore is within a visible range, a colored construct can be obtained and when the light absorption wavelength is outside the visible range, the construct may be used as various electronic materials. Examples of such a chromophore include nitroso, nitro, azo, diarylmethane, triarylmethane, xanthene, acridine, quinoline, methine, thiazole, indamine, indophenol, lactone, aminoketone, hydroxyketone, stilbene, azine, oxazine, thiazine, anthraquinone, phthalocyanine, and indigoid.

As the PHA in the present invention, a random copolymer or a block copolymer comprised of a plurality of the above described monomer units can be used. Therefore, it becomes possible to control physical properties of PHA and add some functions to the PHA by utilizing properties of each monomer unit or functional groups included therein, and also possible to express new functions due to the interaction between functional groups.

In the present invention, it is also possible to utilize a homopolymer constituted of a 3-hydroxypropionic acid unit, a 3-hydroxy-n-butyric acid unit, a 3-hydroxy-n-valeric acid unit, a 4-hydroxy-n-butyric acid unit, or a hydroxyalkanoic acid unit with 6 to 14 carbon atoms, or a copolymer constituted of plural kinds of these units. Also, if necessary, chemical modification or the like may be applied after or in the course of enzymatic synthesis of PHA.

Thus, for example, in the case of forming a construct coated with a PHA of a low affinity to the magnetic material, it is possible to at first coat the magnetic material with a PHA of a high affinity thereto, and then to change the monomer unit composition of such PHA of a high affinity to the magnetic material to a monomer unit composition of a desired PHA in a direction from the interior to the exterior or in a vertical direction, thereby forming a multi-layered structure or a gradient structure, whereby a PHA coating firmly bound to the magnetic material can be obtained.

In the present invention, it is also possible to utilize a PHA in which monomer units constituting a scl-PHA, such as a 3-hydroxypropionic acid unit, a 3-hydroxy-n-butyric acid unit, a 3-hydroxy-n-valeric acid unit and a 4-hydroxy-n-butyric acid unit, and monomer units of mcl-PHA or unusual-PHA described above are present in a mixture. Also, if necessary, chemical modification may be applied after or in the course of synthesis of PHA. The PHA preferably has a number-averaged molecular weight from about 1,000 to 10,000,000.

In the monomer unit of the chemical formula [2], a monomer unit having a carboxyl group (COOR₂₁) as R2 can be produced from a monomer unit represented by the chemical formula [2] and having a vinyl group as R2, namely having a vinylphenyl group at the end of the side chain, by a selective oxidation cleaving of the double bond of the vinyl group into a carboxyl group, whereby a PHA including a monomer unit represented by the chemical formula [2] and having a carboxyphenyl group at the end of the side chain thereof.

The afore-described conversion from a vinyl group to a carboxyl group, namely for obtaining a carboxylic acid by oxidation cleaving of a carbon-carbon double bond with an oxidant is known in various methods such as a method of utilizing a permanganate (J. Chem. Soc., Perkin, Trans. 1, 806 (1973)), a method of utilizing a bichromate (Org. Synth., 4, 698 (1963)), a method of utilizing a periodide (J. Org. Chem., 46, 19 (1981)), a method of utilizing nitric acid (Japanese Patent Application Laid-Open No. S59-190945), a method of utilizing ozone (J. Am. Chem. Soc., 81, 4273 (1959)), etc., and, in relation to PHA, Macromolecular Chemistry, 4, 289-293 (2001) described before reports a method of obtaining a carboxylic acid from a carbon-carbon double bond at the end of the side chain of PHA by a reaction under an acidic condition with potassium permanganate as the oxidant. In executing a reaction of converting the afore-described vinyl group into a carboxyl group in the invention, reference may be made to such an oxidation cleaving method.

In the oxidation cleaving reaction employing potassium permanganate as the oxidant, various inorganic acids, such as sulfuric acid, hydrochloric acid, acetic acid or nitric acid, or organic acids are employed for realizing an acidic condition in the reaction system. However, an acid such as sulfuric acid, nitric acid or hydrochloric acid may cause a cleaving of an ester bond in the main chain of the PHA, thus resulting in a decrease in the molecular weight. For this reason, an acidic condition is preferably realized with acetic acid. An amount of the acid added to the reaction system is usually selected within a range of 0.2 to 200 mol. equivalent with respect to 1 mole of the monomer unit represented by the chemical formula [2] and having a vinyl group as R2, preferably 0.4 to 100 mol. equivalent. An amount of acid added to the reaction system less than 0.2 mol. equivalent results in a low yield of the oxidation cleaving reaction, while an amount exceeding 200 mol. equivalent results in a generation of by-products from the added acid, so that either case is undesirable. Also, a crown ether may be employed for the purpose of accelerating the oxidation cleaving reaction. In such a case, the crown ether and a permanganate salt form a complex to increase the reaction activity. Examples of the crown ether employable for the afore-described purpose include dibenzo-18-crown-6-ether, dicyclo-18-crown-6-ether, and 18-crown-6-ether. An amount of the crown ether to be added to the reaction system is selected within a range of 1.0 to 2.0 mol. equivalent with respect to 1 mole of permanganate salt, preferably 1.0 to 1.5 mol. equivalent.

In executing the afore-described oxidation reaction in the invention, a construct coated with PHA including a unit represented by the chemical formula [2] and having a vinyl group as R2, a permanganate salt and an acid may be collectively charged and reacted in the reaction system from the beginning, or individually charged continuously or intermittently into the reaction system. It is also possible to dissolve or suspend the permanganate salt only in the reaction system, and to add the construct covered with PHA and the acid into the reaction system either continuously or intermittently, or to suspend only the construct covered with PHA in the reaction system and to add the permanganate salt and the acid into the reaction system either continuously or intermittently. It is furthermore possible to at first charge the construct covered with PHA and the acid and to add the permanganate salt either continuously or intermittently, or to at first charge the permanganate salt and the acid and to add the construct covered with PHA either continuously or intermittently, or to at first charge the construct covered with PHA and the permanganate salt and to add the acid either continuously or intermittently.

In the oxidation cleaving reaction to be carried out on the construct covered with a PHA, including a unit represented by the chemical formula [2] and having a vinyl group as R2, utilizing potassium permanganate as an oxidant, a reaction temperature is usually selected within a range of −20 to 40° C., preferably 0 to 30° C. A reaction speed depends on a stoichiometric ratio of the unit represented by the chemical formula [2] and having a vinyl group as R2, and the permanganate salt, and on the reaction temperature, and a reaction time is selected according to a target ratio of conversion of the vinyl group into the carboxyl group, and is usually selected within a range of 2 to 48 hours in the case where such a target ratio is selected at about 100%.

It is also possible, in a unit represented by the chemical formula [1] and having a vinyl group as R1, to convert a vinyl group at an end of the side chain into a carboxyl group, by applying a similar oxidation cleaving reaction.

Also, PHA including a monomer unit represented by the chemical formula [C] or a monomer unit represented by the chemical formula [D] can be prepared by at first producing PHA including a monomer unit represented by the chemical formula [8] and then selectively oxidizing a sulfanyl group (—S—) thereof into a sulfinyl group (—SO—) or a sulfonyl group (—SO₂—). Such a selective oxidation of the sulfanyl group (—S—) can be attained, for example, by an oxidation with a peroxide, and, in such operation, there can be employed any peroxide that can contribute to the oxidation of the sulfanyl group (—S—). In consideration of oxidation efficiency, influence on the skeleton of the main chain of the PHA and on other monomer units contained therein, and simplicity of process, it is particularly preferable to employ a peroxide selected from a group of hydrogen peroxide, sodium percarbonate, m-chloroperbenzoic acid, performic acid and peracetic acid.

For example, as a peroxide for the oxidation of sulfanyl group (—S—), m-chloroperbenzoic acid (MCPBA) allows a stoichiometric oxidation of the sulfanyl group (—S—) present in the monomer unit represented by the chemical formula [8], thereby facilitating control of the proportion of the monomer unit represented by the chemical formula [C] or that represented by the chemical formula [D]. Also, because of mild reaction conditions, there hardly occurs an unnecessary side-reaction, such as cleavage of the main chain skeleton of the PHA or a cross-linking reaction of active sites.

As a general reaction condition for oxidizing a sulfanyl group (—S—) to a sulfinyl group (—SO—), MCPBA is employed in a somewhat excess amount, specifically in an amount of 1.1 to 1.4 moles with respect to 1 mole of the monomer unit represented by the chemical formula [8] and having a sulfanyl group (—S—) in PHA, and a reaction is carried out in chloroform at a temperature of 0 to 30° C. Within the afore-described reaction conditions, the oxidation to sulfinyl group (—SO—) proceeds by about 90% of the theoretical value at a reaction time of about 10 hours, and by about 100% of the theoretical value at a reaction time of about 20 hours. Also, for oxidizing all the sulfanyl group (—S—) into a sulfonyl group (—SO₂—), MCPBA is employed in an amount somewhat in excess of 2 moles, specifically in an amount of 2.1 to 2.4 moles with respect to 1 mole of the monomer unit represented by the chemical formula [8] and having a sulfanyl group (—S—) in the PHA, and a reaction is carried out by selecting a solvent, a temperature and a time similarly as explained above. In such an oxidation process employing MCPBA as the oxidant, a molecule of MCPBA acts on the sulfanyl group (—S—) to convert it into a sulfinyl group (—SO—), and another molecule of MCPBA acts on the sulfinyl group (—SO—) to convert it into a sulfonyl group (—SO₂—), but the conversion from sulfanyl (—S—) to sulfinyl (—SO—) has a higher reaction activity than in the conversion from sulfinyl (—SO—) to sulfonyl (—SO₂—).

Also, a monomer unit represented by the chemical formula [2] and having an epoxy group as R2 can be produced from a monomer unit represented by the chemical formula [2] and having a vinyl group as R2 by a selective oxidation cleaving of the double bond of the vinyl group in a vinylphenyl group at the end of the side chain, thereby introducing an epoxy group. Thus, a PHA including a monomer unit represented by the chemical formula [2] and having a vinyl group as R2 is subjected to a selective oxidation on the vinyl group to provide a PHA represented by the chemical formula [2] and having a 1,2-epoxyethyl group as R2.

Also, in such an oxidation process of epoxylation from the vinyl group to the epoxy group, a peroxide can be utilized, and there can be utilized any peroxide that can contribute to a selective partial oxidation of the vinyl group. In consideration of oxidation efficiency, influence on the skeleton of the main chain of the PHA and on other monomer units contained therein, and simplicity of process, it is particularly preferable to employ a peroxide selected from a group of hydrogen peroxide, sodium percarbonate, m-chloroperbenzoic acid, performic acid and peracetic acid. In the case of employing a peroxide in the epoxylating oxidation from the vinyl group to the epoxy group, reaction conditions can refer to those in the afore-described selective partial oxidation of the sulfanyl group with the peroxide.

The PHA employed in the construct of the present invention, synthesized by the PHA synthetase, is usually an isotactic polymer comprised of R bodies alone.

### 3-hydroxyacyl CoA

3-hydroxyacyl CoA usable as the substrate for the PHA synthetase in the present invention can be, as the substrate for scl-PHA synthetase, 3-hydroxypropionyl CoA, 3-hydroxybutyryl CoA or 3-hydroxyvaleryl CoA, and, as the substrate for mcl-PHA synthetase, 3-hydroxyacyl CoAs represented by the following chemical formulas [11] to [20] and [A′] to [D′].

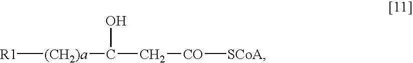

wherein —SCoA represents a coenzyme A bonded to alkanoic acid, in which a combination of R1 and a is at least one selected from a following group, corresponding to the combination of R1 and a in the monomer unit represented by the chemical formula [1]:

a monomer unit in which R1 is a hydrogen atom (H) and a is any of integers from 3 to 10;

a monomer unit in which R1 is a halogen atom and a is any of integers from 1 to 10;

a monomer unit in which R1 is a chromophore and a is any of integers from 1 to 10;

a monomer unit in which R1 is a carboxyl group or a salt thereof and a is any of integers from 1 to 10; and

a monomer unit in which R1 is

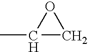

and a is any of integers from 1 to 7.

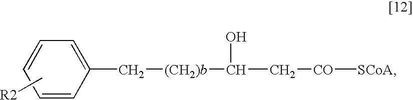

wherein —SCoA represents a coenzyme A bonded to alkanoic acid; b corresponds to b in the monomer unit represented by the afore-described chemical formula [2] and represents any of integers from 0 to 7; and R2 corresponds to R2 in the monomer unit represented by the chemical formula [2] and represents a hydrogen atom (H), a halogen atom, —CN, —NO₂, —CF₃, —C₂F₅, —C₃F₇, a CH₃ group, a C₂H₅ group, a C₃H₇ group, a vinyl group, an epoxy group or COOR21 (R21 representing an H atom, an Na atom or a K atom).

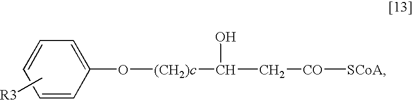

wherein —SCoA represents a coenzyme A bonded to alkanoic acid; c corresponds to c in the monomer unit represented by the afore-described chemical formula [3] and represents any of integers from 1 to 8; and R3 corresponds to R3 in the monomer unit represented by the afore-described chemical formula [3] and represents any one selected from a group of a hydrogen atom (H), a halogen atom, —CN, —NO₂, —CF₃, —C₂F₅, —C₃F₇, a CH₃ group, a C₂H₅ group, a C₃H₇ group and a SCH₃ group.

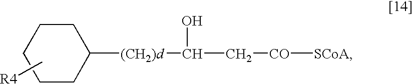

wherein —SCoA represents a coenzyme A bonded to alkanoic acid; d corresponds to d in the monomer unit represented by the afore-described chemical formula [4] and represents any of integers from 0 to 8; and R4 represents any one selected from a group of an H atom, a CN group, a NO₂ group, a halogen atom, a CH₃ group, a C₂H₅ group, a C₃H₇ group, a CF₃ group, a C₂F₅ group, and a C₃F₇ group.

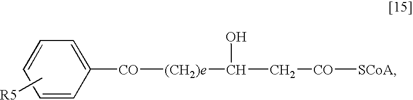

wherein —SCoA represents a coenzyme A bonded to alkanoic acid; e corresponds to e in the monomer unit represented by the afore-described chemical formula [5] and represents any of integers from 1 to 8; and R5 corresponds to R5 in the monomer unit represented by the afore-described chemical formula [5] and represents any one selected from a group of a hydrogen atom (H), a halogen atom, —CN, —NO₂, —CF₃, —C₂F₅, —C₃F₇, —CH₃, —C₂H₅, and —C₃H₇.

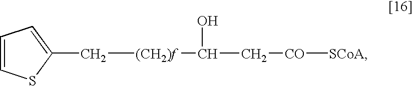

wherein —SCoA represents a coenzyme A bonded to alkanoic acid; and f corresponds to f in the monomer unit represented by the afore-described chemical formula [6] and represents any one of integers from 0 to 7.

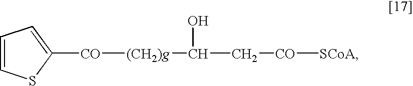

wherein, —SCoA represents a coenzyme A bonded to alkanoic acid; and g corresponds to g in the monomer unit represented by the afore-described chemical formula [7] and represents any one of integers from 1 to 8.

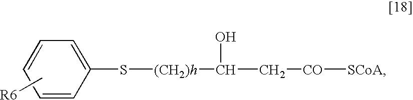

wherein, —SCoA represents a coenzyme A bonded to alkanoic acid; h corresponds to h in the monomer unit represented by the afore-described chemical formula [8] and represents any of integers from 1 to 7; R6 corresponds to R6 in the monomer unit represented by the afore-described chemical formula [8] and represents any one selected from a group of a hydrogen atom (H), a halogen atom, —CN, —NO₂, —COOR′, —SO₂R″, —CH₃, —C₂H₅, —C₃H₇, —CH(CH₃)₂, and —C(CH₃)₃; R′ represents a hydrogen atom (H), Na, K, —CH₃, or —C₂H₅; and R″ represents —OH, —ONa, —OK, a halogen atom, —OCH₃, or —OC₂H₅.

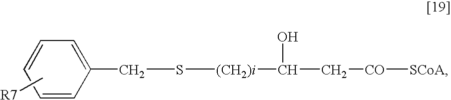

wherein, —SCoA represents a coenzyme A bonded to alkanoic acid, i corresponds to i in the monomer unit represented by the afore-described chemical formula [9] and represents any of integers from 1 to 7; R7 corresponds to R7 in the monomer unit represented by the afore-described chemical formula [9] and represents any one selected from a group of a hydrogen atom (H), a halogen atom, —CN, —NO₂, —COOR′, and —SO₂R″; R′ represents a hydrogen atom (H), Na, K, —CH₃, or —C₂H₅; and R″ represents —OH, —ONa, —OK, a halogen atom, —OCH₃, or —OC₂H₅.

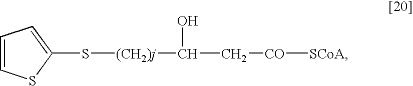

wherein, —SCoA represents a coenzyme A bonded to alkanoic acid, and j corresponds to j in the monomer unit represented by the afore-described chemical formula [10] and represents any of integers from 1 to 9.

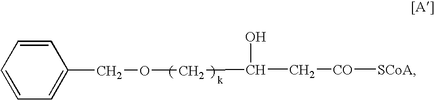

wherein, —SCoA represents a coenzyme A bonded to alkanoic acid, and k corresponds to k in the monomer unit represented by the afore-described chemical formula [A] and represents any of integers from 1 to 8.

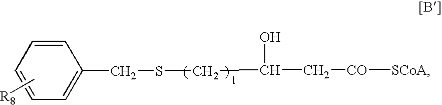

wherein —SCoA represents a coenzyme A bonded to alkanoic acid; 1 corresponds to 1 in the monomer unit represented by the afore-described chemical formula [B] and represents any of integers from 1 to 8; and R₈ corresponds to R₈ in the monomer unit represented by the afore-described chemical formula [B] and represents any one selected from a group of a CH₃ group, a C₂H₅ group, a C₃H₇ group, a CF₃ group, a (CH₃)₂—CH group and a (CH₃)₃-C group.

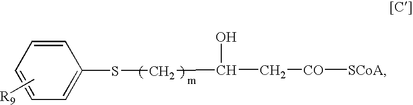

wherein —SCoA represents a coenzyme A bonded to alkanoic acid; m corresponds to m in the monomer unit represented by the afore-described chemical formula [α] and represents any of integers from 1 to 8; and R₉ corresponds to R₉ in the monomer unit represented by the afore-described chemical formulas [α] and [D] and represents an H atom, a halogen atom, a CN group, a NO₂ group, COOR91, SO₂R92 (R91 representing H, Na, K, CH₃ or C₂H₅, and R92 representing OH, ONa, OK, a halogen atom, OCH₃ or OC₂H₅), a CH₃ group, a C₂H₅ group, a C₃H₇ group, a (CH₃)₂—CH group or a (CH₃)₃—C group.

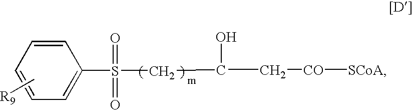

wherein —SCoA represents a coenzyme A bonded to alkanoic acid; m corresponds to m in the monomer unit represented by the afore-described chemical formula [D] and m represents any of integers from 1 to 8; R₉ represents an H atom, a halogen atom, a CN group, a NO₂ group, COOR91, SO₂R92 (R91 representing H, Na, K, CH₃ or C₂H₅, and R92 representing OH, ONa, OK, a halogen atom, OCH₃ or OC₂H₅), a CH₃ group, a C₂H₅ group, a C₃H₇ group, a (CH₃)₂—CH group or a (CH₃)₃—C group; and, in the presence of plural units, the foregoing stands independently for each unit.

These 3-hydroxyacyl CoAs can be synthesized by a suitable method selected from, for example, in vitro synthesis using an enzyme, in vivo synthesis using living organisms, such as microorganisms and plants, and chemical synthesis. Enzymatic synthesis, especially, is commonly used to synthesize these substrates. For example, it is known to use a commercially available acyl CoA synthetase (acyl CoA ligase, E.C.6.2.1.3) to catalyze in the following reaction:

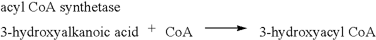

(Eur. J. Biochem., 250, 432-439 (1997), Appl. Microbiol. Biotechnol., 54, 37-43 (2000) etc.). The synthesis process using the enzyme or organism may be a batch process or a continuous process using immobilized enzyme or cells.

### PHA Synthetase and Producing Microorganisms Therefor>

The PHA synthetase used in the present invention can be produced by using a microorganism selected from the microorganisms known to produce a PHA synthetase, or by using a transformant to which the PHA synthetase gene of such a microorganism has been introduced.

Biosynthesis of scl-PHA is an enzymatic polymerization reaction using as a substrate at least one of (R)-3-hydroxypropionyl CoA, (R)-3-hydroxybutyryl CoA and (R)-3-hydroxyvaleryl CoA that is synthesized from various carbon sources through various metabolic pathways in a living organism. In the invention, the enzyme catalyzing the scl-PHA polymerization reaction is called scl-PHA synthetase. Among such scl-PHA synthetases, a PHA synthetase executing a biosynthesis of a PHB is usually called PHB synthetase (also called PHB polymerase or PHB synthase).

The scl-PHA synthetase used in the present invention can be produced by using a microorganism selected from the microorganisms known to produce such a synthetase, or by using a transformant to which the scl-PHA synthetase gene of such a microorganism has been introduced.

For producing scl-PHA synthetase, there can be utilized microorganisms known as PHB or PHB/V producing bacteria. Such a microorganism includes not only those of Aeromonas sp., Alcaligenes sp., Chromatium sp., Comamonas sp., Methylobacterium sp., Paracoccus sp. and Pseudomonas sp., but also Burkholderia cepacia KK01, Ralstonia eutropha TB64 and Alcaligenes sp. strain TL2 separated by the present inventors. The strains KK01, TB64 and TL2 have been deposited, under the Budapest Treaty on the International Recognition of the Deposit of Microorganism for the Purpose of Patent Procedure, under the respective accession numbers: FERM BP-4235, FERM BP-6933 and FERM BP-6913 in International Patent Organism Depositary of Institute of Advanced Industrial Science and Technology (former National Institute of Bioscience and Human Technology, Agency of Industrial Science and Technology), which is located at 1-3, Higashi 1-chome, Tsukaba-shi, Ibaraki-ken, 305 Japan.

In addition to such wild type strains, it is also possible to use a transformant for producing scl-PHA synthetase. Cloning of the scl-PHA synthetase gene, construction of expression vectors and transformants can be done according to the conventional methods. As to the cloning of scl-PHA synthetase gene, the scl-PHA synthetase gene (phbC) of Ralstonia eutropha was cloned. Also, the present inventors have cloned phbC of Burkholderia cepacia KK01 and that of Ralstonia eutropha TB64. The transformant can be prepared by introducing a vector including such phbC into a host. The vector including phbC can be obtained by introducing phbC, for example, into a plasmid vector or a phage vector. As the host, for example, Escherichia coli is often utilized.

Biosynthesis of mcl-PHA and unusual-PHA is also an enzymatic polymerization reaction using as a substrate (R)-3-hydroxyacyl CoA, that is synthesized from various alkanoic acids through various metabolic pathways in vivo (such as β oxidation pathway or fatty acid synthesis pathway). As the microorganism for producing synthetase of mcl-PHA or unusual-PHA (mcl-PHA synthetase), there can be utilized microorganisms known as mcl-PHA or unusual-PHA producing bacteria. Such microorganisms include, in addition to the above described Pseudomonas oleovorans, Pseudomonas resinovorans, Pseudomonas sp. strain 61-3, Pseudomonas putida KT 2442, and Pseudomonas aeruginosa, strains of Pseudomonas sp., such as Pseudomonas putida P91, Pseudomonas cichorii H45, Pseudomonas cichorii YN2, and Pseudomonas jessenii P161, all of which were isolated by the present inventors, strains belonging to Burkholderia sp., such as Burkholderia sp. OK3, FERM P-17370 described in Japanese Patent Application Laid-Open No. 2001-78753 and Burkholderia sp. OK4, FERM P-17371, described in Japanese Patent Application Laid-Open No. 2001-69968. In addition to the above-described microorganisms, it is possible to use microorganisms of genus Aeromonas and Comamonas that can produce mcl-PHA and unusual-PHA.

Strains P91, H45, YN2 and P161 have been deposited with respective accession numbers: FERM BP-7373, FERM BP-7374, FERM BP-7375, and FERM BP-7376 in International Patent Organism Depositary of Institute of Advanced Industrial Science and Technology (former National Institute of Bioscience and Human Technology, Agency of Industrial Science and Technology), which is located at 1-3, Higashi 1-chome, Tsukaba-shi, Ibaraki-ken, 305 Japan.

Microbiological properties of the above-described P91, H45, YN2 and P161 are as follows. As for the strain P161, the base sequence of 16S rRNA is shown as SEQ ID NO: 5.

Bacteriological Properties of Pseudomonas putida P91

### (1) Morphology

Form and size of the cell: rod, 0.6 μm×1.5 μm
Polymorphism of the cell: −

### Mobility: +

Spore formation: −
Gram stain: negative
Colony shape: circular, smooth edge, low convex, smooth surface, lustrous, cream color

### (2) Physiological Properties

Catalase: positive
Oxidase: positive
O/F test: oxidizing type
Reduction of nitrate: negative
Production of indole: negative
Acidification of glucose: negative
Arginine dihydrolase: positive
Urease: negative
Esculin hydrolysis: negative
Gelatin hydrolysis: negative
β-galactosidase: negative
Fluorescent dye production on King's B agar: positive

### (3) Substrate Assimilation

Glucose: positive
L-arabinose: negative
D-mannose: negative
D-mannitol: negative
N-acetyl-D-glucosamine: negative
Maltose: negative
Potassium gluconate: positive
n-capric acid: positive
Adipic acid: negative
dl-malic acid: positive
Sodium citrate: positive
Phenyl acetate: positive

Bacteriological Properties of Pseudomonas cichorii H45

### (1) Morphology

Form and size of the cell: rod, 0.8 μm×1.0 to 1.2 μm
Polymorphism of the cell: −

### Mobility: +

Spore formation: −
Gram stain: negative
Colony shape: circular, smooth edge, low convex, smooth surface, lustrous, cream color

### (2) Physiological Properties

Catalase: positive
Oxidase: positive
O/F test: oxidizing type
Reduction of nitrate: negative
Production of indole: negative
Acidification of glucose: negative
Arginine dihydrolase: negative
Urease: negative
Esculin hydrolysis: negative
Gelatin hydrolysis: negative
β-galactosidase: negative
Fluorescent dye production on King's B agar: positive
Growth in 4% NaCl: negative
Accumulation of poly-β-hydroxybutyric acid: negative

### (3) Substrate Assimilation

Glucose: positive
L-arabinose: negative
D-mannose: positive
D-mannitol: positive
N-acetyl-D-glucosamine: positive
Maltose: negative
Potassium gluconate: positive
n-capric acid: positive
Adipic acid: negative
dl-malic acid: positive
Sodium citrate: positive
Phenyl acetate: positive
Bacteriological Properties of Pseudomonas cichorii YN2

### (1) Morphology

Form and size of the cell: rod, 0.8 μm×1.5 to 2.0 μm
Polymorphism of the cell: −

### Mobility: +

Spore formation: −
Gram stain: negative
Colony shape: circular, smooth edge, low convex, smooth surface, lustrous, translucent

### (2) Physiological Properties

Catalase: positive
Oxidase: positive
O/F test: oxidizing type
Reduction of nitrate: negative
Production of indole: positive
Acidification of glucose: negative
Arginine dihydrolase: negative
Gelatin hydrolysis: negative
β-galactosidase: negative
Fluorescent dye production on King's B agar: positive
Growth in 4% NaCl: positive (weakly growth)
Accumulation of poly-β-hydroxybutyric acid: negative
Hydrolysis of Tween 80: positive

### (3) Substrate Assimilation

Glucose: positive
L-arabinose: positive
D-mannose: negative
D-mannitol: negative
N-acetyl-D-glucosamine: negative
Maltose: negative
Potassium gluconate: positive
n-capric acid: positive
Adipic acid: negative
dl-malic acid: positive
Sodium citrate: positive
Phenyl acetate: positive

Bacteriological Properties of Pseudomonas jessenii P161

### (1) Morphology

Form and size of the cell: spherical: φ 0.6 μm, rod: 0.6 μm×1.5 to 2.0 μm
Polymorphism of the cell: + (elongation)

### Mobility: +

Spore formation: −
Gram stain: negative
Colony shape: circular, smooth edge, low convex, smooth surface, pale yellow

### (2) Physiological Properties

Catalase: positive
Oxidase: positive
O/F test: oxidizing type
Reduction of nitrate: positive
Production of indole: negative
Arginine dihydrolase: positive
Urease: negative
Esculin hydrolysis: negative
Gelatin hydrolysis: negative
β-galactosidase: negative
Fluorescent dye production on King's B agar: positive

### (3) Substrate Assimilation

Glucose: positive
L-arabinose: positive
D-mannose: positive
D-mannitol: positive
N-acetyl-D-glucosamine: positive
Maltose: negative
Potassium gluconate: positive
n-capric acid: positive
Adipic acid: negative
dl-malic acid: positive
Sodium citrate: positive
Phenyl acetate: positive

The PHA producing bacteria as described above may be employed singly or in a combination of two or more kinds, if necessary.

For routine culture of the PHA synthetase-producing microorganisms, for example, to prepare cell stocks, to obtain sufficient cells for enzyme production or to maintain active state of the cells, one can select a suitable culture medium containing ingredients necessary for the growth of the microorganism. Any culture medium can be used as long as it does not interfere with microbial growth or vitality, including common natural media, such as nutrient broth and yeast extracts, and synthetic media supplemented with nutrients.

The culture can be carried out by any culture method, such as liquid culture or solid culture, in which the employed microorganisms can proliferate. Also, there may be employed any of batch culture, fed batch culture, semi-continuous culture or continuous culture. For example, for a liquid batch culture, there can be employed an oxygen supply method by shaking in a shaking flask or by agitated aeration in a jar fermenter. Also, there may be employed a multi-step process in which a plurality of these steps are connected.

To produce a PHA synthetase by using the above-described PHA-producing microorganism, in the case of scl-PHA synthetase, there can be employed a method of growing the microorganism in an inorganic medium containing, for example, a yeast extract, then harvesting the cells in the logarithmic to early stationary growth phase by centrifugation or the like and extracting the enzyme from the cells. Also, in the case of mcl-PHA synthetase, there can be employed a method of growing the microorganism in an inorganic medium containing an alkanoic acid, such as octanoic acid and nonanoic acid, and harvesting the cells in the logarithmic to early stationary growth phase by centrifugation or the like to extract the enzyme from the cells. When cells are cultured as above, scl-PHA derived from the yeast extract etc., or mcl-PHA derived from the added alkanoic acid is synthesized in the cells. In this case, it has been considered that the PHA synthetase exists in a bound form to the fine particles of PHA synthesized in the cell. However, the inventors have found that substantial enzyme activity is present in the supernatant when the cultured cells were disrupted and centrifuged. Presumably, a certain amount of a free PHA synthetase is present because this enzyme is actively synthesized during this relatively early growth phase of the logarithmic to early stationary phase.

Any inorganic culture medium can be used for the above-described culture process as long as the medium contains ingredients such as phosphorus source (phosphate etc.) and nitrogen source (ammonium salt, nitrate etc.) to support microbial growth. Therefore, MSB medium, E medium (J. Biol. Chem., 218, 97-106 (1956)), or M9 medium can be used as the inorganic salt medium, for example. Composition of the M9 medium, which is used in the Examples, is shown in the following.

M9 Culture Medium

| | |
|---|---|
| Na₂HPO₄: | 6.2 g |
| KH₂PO₄: | 3.0 g |
| NaCl: | 0.5 g |
| NH₄Cl: | 1.0 g |

(per liter, pH 7.0).

In addition, it is preferable to add, to the afore-described inorganic culture medium, a stock solution of trace ingredients of the following composition to about 0.3% (v/v), for good proliferation and production of the PHA synthetase.

Trace Ingredient Solution

| | |
|---|---|
| Nitrilotriacetic acid: | 1.5 g |
| MgSO₄: | 3.0 g |
| MnSO₄: | 0.5 g |
| NaCl: | 1.0 g |
| FeSO₄: | 0.1 g |
| CaCl₂: | 0.1 g |
| CoCl₂: | 0.1 g |
| ZnSO₄: | 0.1 g |
| CuSO₄: | 0.1 g |
| AlK(SO₄)₂: | 0.1 g |
| H₃BO₃: | 0.1 g |
| Na₂MoO₄: | 0.1 g |
| NiCl₂: | 0.1 g |

(per liter).

Culture temperature is chosen to be favorable for proliferation of the above strains, so that, for example, it is in the range of 14 to 40° C., preferably about 20 to 35° C.

It is also possible to produce a PHA synthetase by using a transformant to which the PHA synthetase gene of the above PHA-producing strain has been introduced. Cloning of the PHA synthetase gene and construction of expression vectors and transformants can be done according to the conventional methods. To culture a transformant obtained using a bacterial host, such as Escherichia coli, a natural medium such as aN LB medium or a synthetic medium, such as M9 medium, can be used. Cells are cultured with aeration for 8 to 27 hours at 25 to 37° C. After the culture, cells are collected to recover the PHA synthetase accumulated in the cells. Antibiotics, such as kanamycin, ampicillin, tetracycline, chloramphenicol, and streptomycin, may be added to the culture as required. In addition, if an inducible promoter is used in the expression vector, expression may be promoted by adding a corresponding inducer to the culture medium when the transformant is cultured. Such an inducer may be isopropyl-β-D-thiogalactopyranoside (IPTG), tetracycline, or indoleacrylic acid (IAA).

The PHA synthetase may be a cell lysate, a crude enzyme, such as protein components precipitated with ammonium sulfate, or a purified enzyme purified by various methods. The enzyme preparation may be added with a stabilizer or activator, such as metal salts, glycerin, dithiothreitol, EDTA, and bovine serum albumin (BSA) as required.

The PHA synthetase may be isolated and purified by any method as long as the enzyme activity is maintained. For example, the enzyme can be purified as follows: a crude enzyme or ammonium sulfate precipitate thereof, prepared by disrupting microbial cells by using a French press or an ultrasonic homogenizer, lysozyme, or various surfactants and by centrifuging the cell lysate, is purified by affinity chromatography, cation or anion exchange chromatography, gel filtration, or a certain combination thereof. Recombinant proteins, those expressed as a fusion protein having a tag, such as histidine residue at N-terminus or C terminus, can be purified easily by binding through this tag to the affinity resin. The protein of interest may be isolated from the affinity resin binding the fusion protein by treating with a protease, such as thrombin and blood coagulation factor Xa, by lowering the pH, or by adding high concentration imidazole as a binding competitive agent. Alternatively, when pTYB1 (made by New England Biolabs Inc.) was used as an expression vector, and the tag contains inteins, the bonding may be broken under reducing conditions by using dithiothreitol. In addition to the histidine tag, glutathione S-transferase (GST), chitin binding domain (CBD), maltose binding protein (MBP), and thioredoxin are known to allow affinity purification of fusion proteins. The GST fusion protein can be purified by a GST affinity resin.

Enzyme activity of the PHA synthetase can be measured by various known methods. For example, the following method measures CoA released from 3-hydroxyacyl CoA during the PHA polymerization reaction catalyzed by the PHA synthetase utilizing color development with 5,5′-dithiobis-(2-nitrobenzoic acid):

Reagent 1: a 3.0 mg/ml solution of bovine serum albumin (Sigma) dissolved in 0.1 M Tris-HCl buffer (pH 8.0), Reagent 2: a 3.0 mM solution of 3-hydroxyoctanoyl CoA in 0.1 M Tris-HCl buffer (pH 8.0); Reagent 3: a 10 mg/ml solution of trichloroacetic acid in 0.1 M Tris-HCl buffer (pH 8.0), Reagent 4: a 2.0 mM solution of 5,5′-dithiobis-(2-nitrobenzoic acid) in 0.1 M Tris-HCl buffer (pH 8.0). First reaction (PHA synthesizing reaction): 100 μl of Reagent 1 is added to and mixed with 100 μl of the sample (enzyme) solution, then the mixture is pre-incubated for one minute at 30° C., to which 100 μl of Reagent 2 is added and mixed. The resultant mixture is incubated for 1 to 30 minutes at 30° C., and the reaction is stopped by adding Reagent 3. Second reaction (color development of free CoA): the resulting first reaction solution is centrifuged (15,000×g, for 10 minutes). To 500 μl of the supernatant, 500 μl of Reagent 4 is added and incubated for 10 minutes at 30° C. Then, the absorbance at 412 nm is measured. Calculation of enzyme activity: the amount of enzyme that releases 1 μmol of CoA within one minute is defined as one unit (U).

Also, in the case of scl-PHA, a similar measurement is possible by employing s-hydroxybutyryl CoA instead of 3-hydroxyoctanoyl CoA described above.

Generally, the PHA synthesized by the above described enzyme is an isotactic polymer made with R bodies alone.

Magnetic Material

In the present invention, any magnetic material capable of immobilizing the PHA synthetase can be suitably selected and used. Also, the kind and the structure of the magnetic material can be suitably selected according to the immobilizing method for the PHA synthetase and the form of application of the prepared construct.

A magnetic material constituting the construct of the invention can be, for example, a metal or a metal compound having magneticitiy, more specifically, magnetite (Fe₃O₄), γ-hematite (γ-Fe₂O₃), a ferrite, such as MnZn ferrite, NiZn ferrite, YFe garnet, GaFe garnet, Ba ferrite or Sr ferrite, a metal, such as iron, manganese, cobalt, nickel or chromium, or an alloy such as of iron, manganese, cobalt or nickel, and such examples are not restrictive. In the case of immobilizing a biological substance or administering a magnetic construct into a living organism, there can be advantageously employed, in addition to magnetite (Fe₃O₄) having satisfactory biocompatibility, a ferrite composition formed by substituting a part of the metal element of the magnetite with at least another metal element. Such a magnetic material has a shape that varies depending on the generation conditions, e.g., polyhedral, octahedral, hexahedral, spherical, rod-shaped or flake-shaped, but a shape with low anisotropy is preferable for a stable expression of functionality. A primary particle size of the magnetic material constituting the construct of the invention can be suitably selected according to the application thereof, but it is, for example, preferably within a range of 0.001 to 10 μm.

As the magnetic material of the invention, a material showing super paramagnetism can also be employed advantageously. For example, a ferrite having a particle size as small as about 20 nm or less shows super paramagnetism under thermal perturbation to lose retentive magnetization or coercive force. A material with super paramagnetism can be magnetically manipulated by applying a magnetic field, and is free from magnetic coagulation in the absence of the magnetic field due to the absence of retentive magnetization or coercive force.

The magnetic material can also be a composite material, such as a matrix including a metal or a metal compound, and such a matrix can be constituted of various organic or inorganic materials. Also, a material formed by coating a surface of an organic polymer with a magnetic material, for example, by ferrite plating, or a material formed by dispersing a magnetic material in an organic polymer, can also be utilized in the case where the magnetic material is exposed in a part of the surface.

Furthermore, a magnetic material subjected to a hydrophobic treatment by various methods, such as a method of coating the particle surface with a fatty acid or a treatment with various coupling agents, represented by a silane coupling agent, can also be employed as the magnetic material of the invention.

Preparation of Construct

A method for producing the construct of the invention includes a step of immobilizing the PHA synthetase on the magnetic material, and a step of reacting 3-hydroxyacyl CoA with thus immobilized PHA synthetase to synthesize the PHA.

For immobilizing the PHA synthetase to the magnetic material, an ordinary enzyme immobilizing method may be arbitrarily selected as long as it can ensure the activity of the enzyme and is applicable to the desired magnetic material. Examples of such a method include a covalent bonding method, an ionic adsorption method, a hydrophobic adsorption method, a physical adsorption method, an affinity adsorption method, a cross-linking method, and a lattice inclusion method, but the immobilizing method utilizing ionic adsorption or hydrophobic adsorption is particularly simple and convenient.

An enzyme protein, such as that of the PHA synthetase, is a polypeptide formed by a plurality of amino acids and shows the properties of an ionic adsorbing material due to an amino acid having an ionic group on the side chain, such as lysine, hystidine, arginine, aspartic acid, or glutamic acid, and also shows the properties of a hydrophobic adsorbing material due to an amino acid having a hydrophobic side chain, such as alanine, valine, leucine, isoleucine, methionine, tryptophane, phenylalanine or proline, and due to the organic polymer structure. Therefore, it can adsorb, though at a variable level, to a solid surface having an ionic property and/or a hydrophobic property.

Among the magnetic material in the invention, a metal oxide, such as ferrite, has hydroxyl groups on the surface, and they can be advantageously immobilized through a hydrogen bond with carboxyl groups at the surface of the PHA synthetase.

The fixing of the PHA synthetase to a core magnetic material by an ionic adsorption method or a hydrophobic adsorption method can be achieved by mixing the enzyme and the core in a predetermined reaction liquid. In this operation, it is preferable to shake or agitate a reaction vessel with a suitable intensity in order for the enzyme to be uniformly adsorbed on the surface of the core.

As the polarity and amount of the surface charge and the hydrophobicity of the core and the PHA synthetase vary depending on the pH, salt concentration and temperature of the reaction liquid, it is desirable to regulate the solution within a permissible range, according to the properties of the core to be employed. For example, in the case where the core principally shows an ionic adsorption property, it is possible to increase the charge amount, which contributes to the adsorption between the core and the PHA synthetase, by reducing the salt concentration. It is also possible to increase opposite charges on both components by a change in the pH. Also, in the case where the core principally shows a hydrophobic adsorption property, the hydrophobicity of both components can be increased by an increase in the salt concentration. It is also possible to investigate the charge state or hydrophobicity of the core and PHA synthetase by electrophoresis or wet angle measurement in advance, and to select solution conditions suitable for the adsorption. Furthermore, such conditions can be determined by a direct measurement of the adsorbed amount of the PHA synthetase by the core. The adsorption amount can be measured, for example, by adding a solution of the PHA synthetase of a known concentration to a core dispersion, then, after an adsorption process, measuring the concentration of the PHA synthetase in the solution and determining the amount of the adsorbed enzyme by subtraction.

In the case of a core on which it is difficult to immobilize the enzyme by the ionic adsorption or the hydrophobic adsorption, a covalent bond method may be adopted if the operation is not too cumbersome and a possibility of enzyme inactivation can be tolerated.

It is furthermore possible to fuse the PHA synthetase to a peptide including an amino acid sequence having a binding ability to the magnetic material to immobilize the PHA synthetase on the surface of such a magnetic material based on a binding property between the peptide portion of the amino acid sequence having the binding ability to the magnetic material and the magnetic material.

An amino acid sequence having a binding ability to the magnetic material can be determined, for example, by a random screening of a peptide library. For example, there can be employed a phage display peptide library prepared by fusing a product of a random synthetic gene to the N-terminus of a surface protein (for example, gene III protein) of an M13 type phage, and, in such a case, an amino acid sequence having a binding ability to the magnetic material can be determined by the following procedure. The phage display peptide library is brought into contact with the magnetic material or a component thereof, and then bound phages and not bound phages are separated by washing. The phages binding to the magnetic material can be eluted, for example, with an acid, then neutralized with a buffer and Escherichia coli is infected therewith to amplify the phages. In this selection, after being repeated several times, plural clones having a binding ability to the object magnetic material are concentrated. Then, in order to obtain a single clone, Escherichia coli is infected again to form colonies on a culture medium plate. After each colony is cultured in a liquid medium, the phages present in the supernatant are precipitated and purified, for example, with polyethylene glycol, and are subjected to a base sequence analysis to determine the structure of the peptide.

Thus obtained amino acid sequence of the peptide having a binding ability to the magnetic material is utilized by fusing it to the PHA synthetase by an ordinary genetic engineering method. The peptide having the binding ability to the magnetic material can be expressed as a fusion to the N-terminus or C-terminus of the PHA synthetase. It can be expressed with an inserted suitable spacer sequence. The spacer sequence preferably includes from about 3 to 400 amino acids, and may include any amino acid. A most preferred spacer sequence does not hinder the function of the PHA synthetase and the binding thereof to the magnetic material.

The enzyme-immobilized magnetic material prepared utilizing the afore-described method may be used in such a prepared state or after a lyophilization, etc.

By defining 1 unit (U) of the amount of the PHA synthetase releasing 1 μmol/minute of CoA in the PHA synthesis reaction by polymerization of 3-hydroxyacyl CoA, the amount of the enzyme to be used in the reaction is selected within a range of 10 to 1,000 Upper 1 g of magnetic material, preferably 50 to 500 U.

The construct in which the magnetic material is covered by the PHA is prepared by charging the enzyme-immobilized magnetic material into an aqueous reaction liquid containing a 3-hydroxyacyl CoA, constituting the raw material of the desired PHA, and causing the PHA synthetase on the surface of the magnetic material to synthesize the PHA. The aqueous reaction liquid should be constructed as a reaction system adjusted to the conditions capable of exhibiting the activity of the PHA synthetase, and is prepared with a buffer normally within a pH range of 5.5 to 9.0, preferably 7.0 to 8.5. However the conditions may be set outside the afore-described range, depending on the optimum pH or pH stability of the PHA synthetase to be employed. Such a buffer can be selected suitably according to the desired pH range, as long as the activity of the employed PHA synthetase can be exhibited, but there can be advantageously employed ordinary buffer utilized in the biochemical reactions, such as acetic acid buffer, phosphoric acid buffer, potassium phosphate buffer, 3-(N-morpholino)propane sulfonic acid (MOPS) buffer, N-tris(hydroxymethyl)methyl-3-aminopropane sulfonic acid (TAPS) buffer, trishydrochloric acid buffer, glycine buffer, 2-(cyclohexylamino)ethane sulfonic acid (CHES) buffer etc. The concentration of the buffer is not particularly limited as long as the activity of the employed PHA synthetase can be exhibited, but is advantageously selected within a range of 5.0 mM to 1.0 M, preferably in a range of 0.1 to 0.2 M. The reaction temperature is suitably selected according to the characteristics of the PHA synthetase to be employed, but is normally selected within the range of 4 to 50° C., preferably 20 to 40° C. However the conditions may be set outside the afore-described range, depending on the optimum temperature or the heat resistance of the PHA synthetase to be employed. The reaction time, though dependent on the stability of the PHA synthetase to be employed, is normally within the range of 1 minute to 24 hours, more desirably 30 minutes to 3 hours. The concentration of 3-hydroxyacyl CoA in the reaction liquid is suitably selected within a range capable of exhibiting the activity of the PHA synthetase to be employed, but is normally selected within the range of 0.1 mmol/L to 1.0 mol/L, preferably 0.2 mmol/L to 0.2 mol/L. Since the pH of the reaction liquid tends to decrease when the concentration of 3-hydroxyacyl CoA in the reaction liquid is high, it is preferable to have a higher concentration in the afore-described buffer if a high concentration is selected for 3-hydroxyacyl CoA.

Also, a compound having a hydroxyl group may be suitably added to the reaction liquid in view of controlling the molecular weight of the PHA and improving the hydrophilicity of the PHA coating film.

The compound having the hydroxyl group to be used in the method of the invention is at least one selected from an alcohol, a diol, a triol, an alkylene glycol, a polyethylene glycol, a polyethylene oxide, an alkylene glycol monoester, a polyethylene glycol monoester, and a polyethylene oxide monoester, and is selected as explained more specifically in the following. The alcohol, diol or triol has a linear or ramified structure with 3 to 14 carbon atoms. The alkylene glycol or alkylene glycol monoester has a linear or ramified carbon chain with 2 to 10 carbon atoms. The polyethylene glycol, polyethylene oxide, polyethylene glycol monoester, or polyethylene oxide monoester has a number-averaged molecular weight within a range from 100 to 20,000.

Such the concentration of a compound having the hydroxyl group is not particularly limited as long as it does not hinder the polymerization reaction of 3-hydroxyacyl CoA by the PHA synthetase, but this compound is preferably added in an amount of 0.01 to 10% (w/v) with respect to the reaction liquid of the PHA synthetase and 3-hydroxyacyl CoA, more preferably 0.02 to 5% (W/v), and may be added either collectively at an early stage of the reaction or in several portions during the reaction time.

Also, in the above-described process, by varying in time the composition, such as type and concentration of the 3-hydroxyacyl CoA in the aqueous reaction liquid, the monomer unit composition of the PHA constituting the particulate construct can be varied in a direction from the inner side to the outer side, in the case of a particulate construct, or in a perpendicular direction, in the case of a flat construct.

In such a construct showing a change in the monomer unit composition, there can be realized a configuration in which the single-layered PHA covers the magnetic material with a continuous change in the composition, thus forming a gradient composition in the direction from the inner side to the outer side or in the perpendicular direction. Such a configuration can be realized, for example, in the course of the synthesis of the PHA, by adding 3-hydroxyacyl CoA of another composition.

In another configuration, the PHA film has step-wise changes in the composition and the magnetic material is covered by plural layers of the PHA with different compositions. Such a configuration can be realized, for example, by synthesizing a PHA with a certain composition of 3-hydroxyacyl CoA, then collecting the construct under preparation from the reaction liquid, for example, by centrifuging, and adding again a reaction liquid having a different composition of 3-hydroxyacyl CoA.

The construct obtained in the afore-described reaction is subjected, if necessary, to a washing step. The washing method for the construct is not particularly limited as long as it does not provide the construct with a change undesirable for the object of preparation of the construct. In the case of a capsule construct with the magnetic material as a core and the PHA as an outer coating, it is possible to eliminate unnecessary components contained in the reaction liquid by precipitating the construct by centrifuging and removing the supernatant. Further washing is possible by adding a washing agent, such as water, a buffer or methanol in which the PHA is insoluble, and executing centrifugation. Also, filtration or the like may be employed in lieu of centrifuging. In the case where the construct is formed by coating a flat-shaped magnetic material with the PHA, it can be washed by immersion in the afore-described washing agent. Further, the construct may be subjected to a drying step, if necessary, or to various secondary processes or chemical modification.

For example, by applying a chemical modification to the PHA that covers the magnetic material, there can be obtained a construct having more useful functions and characteristics.

For example, by introducing a graft chain, there can be obtained a construct in which at least a part of the magnetic material is covered with the PHA having various characteristics, derived from such a graft chain. Also, by cross-linking the PHA, it is possible to control mechanical strength, chemical resistance, heat resistance, etc., of the construct.

The chemical modification method is not particularly limited as long as it can attain the desired functions and structure, but there can be advantageously employed a method of synthesizing a PHA having a reactive functional group in the side chain and executing a chemical modification utilizing the chemical reaction of such a functional group.

The type of the afore-described reactive functional group is not particularly limited as long as it can attain the desired functions and structure, but the afore-described epoxy group can be cited as an example. The PHA having an epoxy group in the side chain can be subjected to a chemical conversion as in the ordinary polymer having an epoxy group. More specifically, there can be carried out a conversion into a hydroxyl group or introduction of a sulfone group. It is also possible to add a compound having thiol or amine, and, more specifically, a graft chain of the polymer can be formed by a reaction under the addition of a compound having an end amino group highly reactive with the epoxy group.

Examples of the compound having an amino group at the end include amino-modified polymers, such as polyvinylamine, polyethylenimine or amino-modified polysiloxane (amino-modified silicone oil). Among these, amino-modified polysiloxane can be commercially available modified silicone oil or can be synthesized by the method described, for example, in J. Amer. Chem. Soc., 78, 2278 (1956), and is expected to provide effects by the addition of a graft chain in the polymer, such as an improvement in the heat resistance.

Also, a ligand-receptor reaction is widely utilized as a highly sensitive reaction technology. The ligand-receptor reaction includes reactions utilizing various specific binding, such as an antigen-antibody reaction, a complimentary property of nucleic acid, or a physiologically active substance and a receptor thereof, such as hormone-receptor, enzyme-substrate, biotin-avidin, etc. Such a reaction is generally carried out by binding the ligand or the receptor with a carrier, then executing a ligand-receptor reaction, and separating the counterpart receptor or ligand from the medium. This reaction is widely utilized in a purification method for separating and purifying an antibody, a hormone, or a nucleic acid of a specified sequence, present in a trace amount in a medium, or a ligand-receptor assay for detecting such a substance.

The reactive functional group of the PHA of the invention can be advantageously utilized for supporting a ligand or a receptor to be used in such a ligand-receptor reaction, and useful functions and characteristics can be expressed by the grafting.

Other examples of chemical conversion of the polymer having an epoxy group include a cross-linking reaction with a diamine compound, such as hexamethylene diamine, succinic anhydride or 2-ethyl-4-methylimidazole, and examples of physicochemical conversion include a cross-linking reaction by electron beam irradiation. Among these, the reaction between the PHA having an epoxy radical in the side chain and hexamethylene diamine proceeds in the following manner to produce cross-linked polymer:

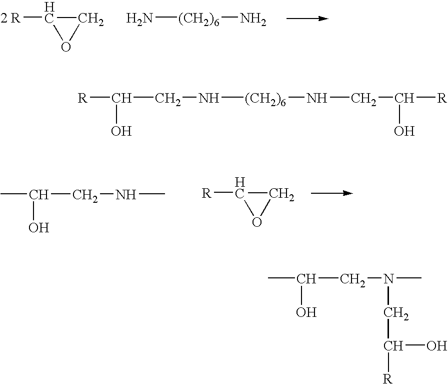

The construct of the invention includes the magnetic material in an amount of 1 to 80 wt. %, preferably 5 to 70 wt. %, and more preferably 10 to 60 wt. %. If the amount of the magnetic material is less than 1 wt. %, the magnetic properties may be insufficient, leading to a magnetic material-containing construct of an insufficient performance. Conversely, if the amount of the magnetic material exceeds 80 wt. %, the functionality of the construct itself may deteriorate, because of the excessive amount of the magnetic material, thus resulting in an unsatisfactory practical performance.

A particle size of the construct of the invention is suitably selected according to the application, etc., but is usually 0.02 to 100 μm, preferably 0.05 to 20 μm.

Also, a thickness of the coating film of the layered construct of the invention is suitably selected according to the application, etc., but is usually 0.02 to 100 μm, preferably 0.05 to 20 μm.

In the case where the construct of the invention is used for immobilizing a bio-related substance or for administration into a living organism, a configuration in which the magnetic material is completely covered with the PHA is more preferred in order to minimize the elution of the magnetic material and inhibition of the interaction of the bio-related substance.

In the obtained construct, the coating of the magnetic material by the PHA can be confirmed by a method of combining a composition analysis, for example, by gas chromatography and a morphological observation under an electron microscope, or a method of judging the structure by employing time of flight secondary ion mass spectrometer (TOF-SIMS) and ion sputtering, thereby judging the structure from the mass spectrum of each constituent layer. However, for a more direct and simpler confirmation, there can be employed a method of combining dyeing with Nile blue A and observing under a fluorescence microscope developed newly by the present inventors. As a result of the intensive investigation for a simple evaluation of the PHA synthesis in a cell-less (in vitro) system, the present inventors have found that Nile blue A, which generates fluorescence via a specific bonding with the PHA and is reported, in Appl. Environ. Microbiol., 44, 238-241 (1982), to be usable for simple judgment of an in vivo PHA production, can also be used for analyzing the PHA synthesis in the cell-less system by selecting a suitable method and conditions of use, and have reached the above-described method. In this method, the PHA synthesis in the cell-less system can be easily judged by mixing the Nile blue A solution of a predetermined concentration, after filtration, with the reaction liquid containing the PHA, irradiating excitation light of a predetermined wavelength and observing the fluorescence generated from the synthesized the PHA alone under a fluorescence microscope. This method, applied to the preparation of the construct of the present invention, allows to directly observe and evaluate the PHA coating the surface of the hydrophobic solution, unless the used magnetic material generates fluorescence under the afore-described conditions.

Also, the distribution of the composition of the PHA that covers the magnetic material, in the direction from the inner side to the outer side, or in the perpendicular direction, can be evaluated by combining an ion sputtering technology and time of flight secondary ion mass spectroscopy (TOF-SIMS).

Utilization of Construct

One of the goals of the invention is to enable the manufacture of a construct that has been difficult to prepare with the ordinary organosynthetic chemical methods, and it is now possible to obtain a construct with excellent characteristics that could not be produced with a capsule construct or a layered construct prepared using the conventional organosynthetic chemical methods. For example, the present invention makes it possible to utilize a novel polymer compound or to provide a novel function and structure, which have been difficult to realize in the conventional organosynthetic methods. More specifically, utilizing an extremely strict molecule identifying ability or stereoselectivity specific to the biological catalytic action, a capsule construct or a layered construct coated with a functional polymer compound or a polymer compound of an extremely high chirality, difficult to realize in the conventional organosynthetic methods, can be produced with an extremely simple process.

In particular, it is possible to prepare a construct coating a magnetic material of excellent dispersion uniformity by an extremely simple process without applying subjecting a metal or a metal compound having magneticity to an oleophilic treatment.

It is further possible to prepare, in an extremely simple process, a capsule construct, which covers a magnetic material and which shows excellent dispersibility for the magnetic material, excellent magnetic response, and low elution of metal ions to the exterior. Therefore, this process is applicable to various applications and fields.

The construct of the invention is practically free from being influenced by the elution of the magnetic material during use, since the magnetic material is substantially absent or present only in an extremely small amount on the surface and/or the vicinity thereof of the particle. Therefore, the construct of the invention can be used in the same manner as conventional non-magnetic particles, even in biochemical applications in which a metal component is often avoided, and can be employed, for example, for supporting antigens, antibodies, proteins, nucleic acids, etc., of a wide range as a carrier for ordinary diagnostic drugs and a carrier for drug delivery with a small side effect. Also, it can be used as a carrier for a diagnostic drug in an enzymatic immunoassay, suppressing a non-specific color development of the enzyme resulting from elution of the magnetic material, thus being usable in various detecting methods and showing an extremely high practical performance. Furthermore, the construct of the invention can also be used as a nucleic acid capturing member by supporting a specific nucleic acid or a protein probe for capturing a specified nucleic acid on the surface of the particles. For such applications, the conventional magnetic material-containing polymer particle cannot be used in the PCR method since a metal component, particularly iron, hinders the PCR reaction. On the other hand, the construct of the invention, in which the surface-exposed magnetic material is practically absent, does not hinder the PCR reaction, and can therefore be used for the PCR method in a state supporting the captured nucleic acid. Therefore, the construct of the invention can be used extremely advantageously in wide technical fields including inspection, diagnostic and therapeutic fields utilizing nucleic acid and industrial fields utilizing nucleic acid.

However, the construct of the invention, the methods of utilizing thereof and the producing method therefor are not limited to those explained in the foregoing.

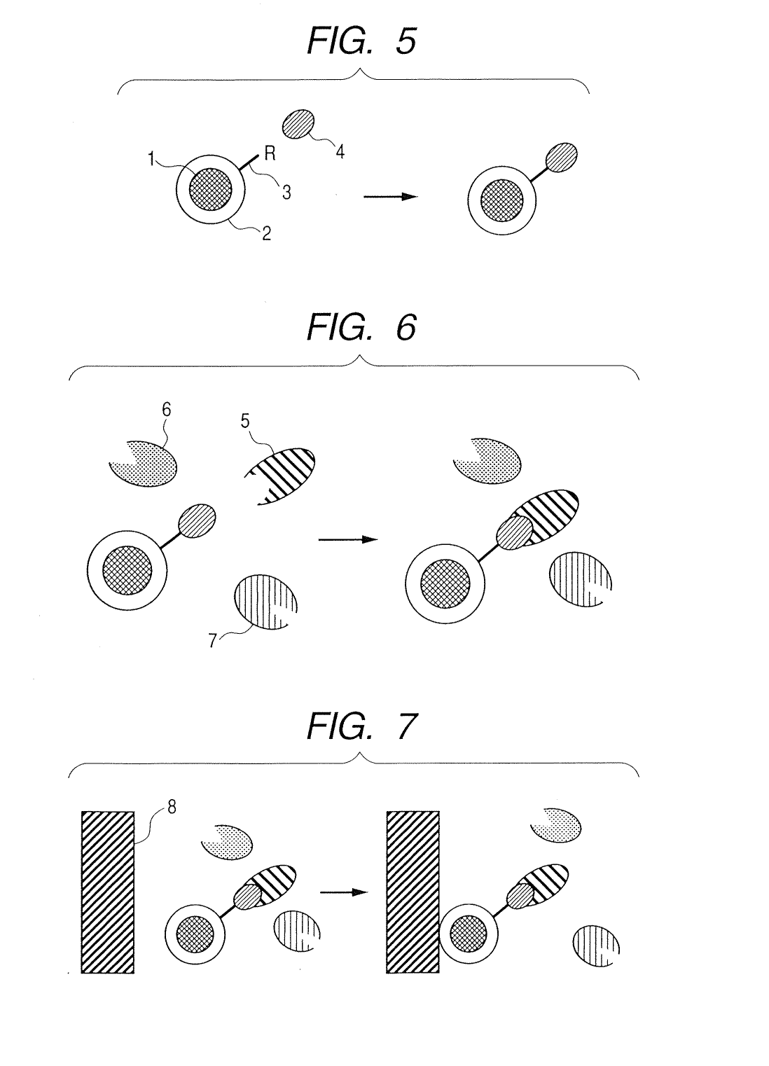

Methods for Separation/Recovery, Detection and Screening of Target Component First, features of the present invention will be explained with reference to FIGS. 5 to 7.

In the method for separation/recovery, detection and screening of a target component of the invention, a molecule having a binding affinity to the target component is utilized as a means for selectively obtaining the target component, in a state of a construct in which the molecule 4 having a binding affinity to the target component (target component-binding molecule) is borne and immobilized in advance on the surface of a carrier. More specifically, the carrier to be employed has a structure including a coating layer of an organic polymer material on the surface of a base material of the carrier. For example, a base material constituted of a magnetic material is employed as the carrier base material 1, and at least a part of the surface of the carrier base material 1 is covered by polyhydroxyalkanoate 2, which is a polymer material of a high biological affinity, as the coating layer of the organic polymer material, provided on the surface of the base material. In the case where the carrier base material contains a magnetic material, a magnetic carrier having a coating of a polyhydroxyalkanoate (also represented as PHA) is also represented as a “PHA magnetic construct.” For immobilizing the target component-binding molecule 4 on a carrier having a coating with polyhydroxyalkanoate 2, such as the PHA magnetic construct, there is utilized a site 3, present in the coating of polyhydroxyalkanoate 2, that can selectively hold the target component-binding molecule 4. By employing a base material of a magnetic material as the carrier base material 1, a PHA magnetic construct carrying the target component-binding molecule can be prepared by the step of binding the target component-binding molecule 4 on such a PHA magnetic construct.

Then, there is carried out a step of mixing the PHA coated carrier carrying the target component-binding molecule with a liquid containing a target component 5 dissolved or dispersed therein, namely, a specimen or a mixed sample, and, after a step of contacting the target component 5 contained in the specimen or the mixed sample with the target component-binding molecule 4 carried on the carrier, the target component-binding molecule 4 binds the target component 5, whereupon the target component 5 is immobilized on the carrier. On the other hand, other components 6, 7 contained in the specimen or the mixed sample do not bind the target component binding component 4 and show little non-specific attachment to the PHA, which covers the carrier surface. Even if such components 6, 7 other than the target component cause non-selective binding, they can be easily removed by a simple washing operation.

After the target component 5 is fixed on the carrier, the carrier is recovered and separated, for example, by solid-liquid separating means, whereby the carrier on which the target component 5 is immobilized is recovered and separated. Also, in the case of a carrier utilizing a magnetic material as the carrier base material 1, through a magnetic field applied by a magneticity-generating structured member 8 (such as a permanent magnet or an electromagnet), the carrier is collected on the structured member 8 by a magnetic attractive force between the base material of the magnetic material and the structure member 8 generating the magnetic force.

In the method for separation/recovery, detection and screening of target component of the invention, as explained in the foregoing, the target component-binding molecule is held by a polyhydroxyalkanoate, which is a polymer material of a high biological affinity, and the binding of the target component and the target component-binding molecule also takes place on the surface portion coated with the polyhydroxyalkanoate, so that the separation/recovery, detection and screening of target component can be carried out under conditions close to those of a living organism.

The method for separation/recovery, detection and screening of a target component of the invention may be applied to a specimen where components other than the target component are not present. In the case where the target component is present alone in the specimen but at a low concentration in a large volume, such a target component is immobilized to the carrier and is recovered with the carrier, whereby the target component is present at a higher proportion in the separated and recovered carrier and is thus concentrated, thereby facilitating the detection.

The polyhydroxyalkanoate 1 is already explained in detail above in the PHA section, and the magnetic material 2 in the Magnetic Material section, the molecule 4 having a binding affinity to the target component and the target component 5 will be explained below in a section entitled Target Component and Molecule Having Binding Affinity to Target Component; and magnetic member 8 in a section entitled Magnetic Separation and Washing of Target Component Binding Magnetic Construct.

Also, a compound having a hydroxyl group may be suitably added to the reaction liquid in view of controlling the molecular weight of the PHA and improving the hydrophilicity of the PHA coating film.

The compound having the hydroxyl group to be added to the reaction liquid in the method of the invention is at least one selected from an alcohol, a diol, a triol, an alkylene glycol, a polyethylene glycol, a polyethylene oxide, an alkylene glycol monoester, a polyethylene glycol monoester, and a polyethylene oxide monoester, and is selected as explained more specifically in the following. The preferred alcohol, diol or triol has a linear or ramified structure with 3 to 14 carbon atoms. The preferred alkylene glycol or alkylene glycol monoester has a linear or ramified carbon chain with 2 to 10 carbon atoms. The preferred polyethylene glycol, polyethylene oxide, polyethylene glycol monoester, or polyethylene oxide monoester has a number-averaged molecular weight within a range from 100 to 20,000.

Such the concentration of a compound having the hydroxyl group is not particularly limited as long as it does not hinder the polymerization reaction of 3-hydroxyacyl CoA by the PHA synthetase, but the compound is preferably added in an amount of 0.01 to 10% (w/v) with respect to the reaction liquid containing the PHA synthetase and 3-hydroxyacyl CoA, more preferably 0.02 to 5% (w/v), and may be added either collectively at an early stage of the reaction or in several portions during the reaction time.

The construct including a magnetic material as a core, to be used in the method of the invention, includes the magnetic material in an amount of 1 to 80 wt. %, preferably 5 to 70 wt. %, and more preferably 10 to 60 wt. %. If the amount of the magnetic material in the construct is less than 1 wt. %, the magnetic properties may be insufficient, leading to a magnetic material-containing construct of an insufficient performance. Conversely, if the amount of the magnetic material exceeds 80 wt. %, the functionality of the construct itself achieved by a sufficient PHA coating layer on the surface may deteriorate, because of a relative decrease of the content of the PHA coating the surface of the magnetic material constituting the core, thus resulting in an unsatisfactory practical performance.

A particle size of the particulate construct to be used in the method of the invention is suitably selected according to the individual application, etc., but is usually selected within a range of 0.02 to 100 μm, preferably 0.05 to 20 μm.

Target Component, and Molecule Having a Binding Affinity to Target Component

A “target component”, being an object of the method of the invention, is physiologically active and is often present in a specimen, as a mixture with another substance, or, singly, without other substances, at a low concentration in a large volume. It is therefore desired to obtain a method of separating/recovering, detecting or screening only the “target component” in the specimen.

The method of the invention utilizes, for the afore-described objective, a “molecule having a binding affinity to the target component (hereinafter also represented as “target component-binding molecule”)” advantageously utilizable for capturing the “target component” only.

Specific examples of the “target component” and the “molecule having binding affinity to the target component,” considered in the method of the present invention, include a nucleic acid, a protein, a peptide, a sugar chain, a lipid, a low-molecular compound, a composite thereof, and a substance containing such a substance as a portion.

“Nucleic acid” includes a deoxyribonucleic acid, a ribonucleic acid, an oligonucleotide, a polynucleotide, an aptamer, and a ribozyme.

“Protein” includes natural and artificial irregular molecules such as a glycoprotein, a lipoprotein, a membrane protein, a labeling protein, or a low-molecular peptide.

In the case where such a protein is immunoreactive, there are included, for example, an antibody, an antigen, a haptene, and a complex thereof, and, particularly in the case where it is an antibody, there are included a monoclonal antibody, a polyclonal antibody, a recombinant protein antibody, a natural antibody, a chimeric antibody, a hybrid antibody mixture (single or plural), a single chain antibody expressing a phage antibody (including the entire phages expressing single-chain antibodies), and an antibody-protein fusion, and a hybrid mixture thereof. In the case where the protein is a catalytic reactive member, there are included a natural enzyme, a modified enzyme prepared by genetic engineering, a semi-artificial enzyme formed by complexing with a synthetic molecule, such as polyethylene glycol, and a semi-artificial enzyme in which a non-natural amino acid is introduced.

The “antibody” is usually exemplified by IgG (immunoglobulin G), but there can also be employed substances of a lower molecular weight, such as F(ab′)₂, Fab′, Fab or Fv obtained by treating with a proteolytic enzyme, such as pepsin and papain, or a reducing agent, such as dithiothreitol. In addition to IgG, there can also be employed IgM or a fragment of a lower molecular weight of IgM obtained by a process similar to that for IgG. Furthermore, a monoclonal antibody or a polyclonal antibody can be used as the “target component-binding molecule” in the invention. In the case of employing the monoclonal antibody as the “target component-binding molecule” of the invention, for a protein having a repetitive structure, such as a surface antigen of hepatitis B virus or for an antigen containing plural epitopes within the molecule as in a CA19-9 antigen, there may be employed plural monoclonal antibodies, reactive to the respective epitopes, in combination. It is also possible to utilize two or more different identifying epitopes in a combination.

On the other hand, “antigen” includes various substances, such as a protein, a polypeptide, a steroid, a polysaccharide, a lipid, a pollen, a recombinant protein produced by a genetic engineering method, a drug, etc. Thus, the “antigen” considered in the invention includes, among all the substances capable of inducing an antibody production in a human or in an animal, single or multiple substances selected for a particular objective, such as diagnosis, and a mixture containing the same.

“Peptide” in the invention indicates a fragment of a protein regardless of the molecular weight thereof.

“Low-molecular compound” is a molecule, preferably an organic molecule, of a low molecular weight, recognizable by a receptor. The low-molecular compound is usually a compound capable of a specific binding with a protein and is often a physiologically active substance or a drug candidate. In particular, a low-molecular compound with an antigen property may also be called “hapten.” “Sugar chain” includes an oligomer or a polymer of linear or branched structure, formed by a chain of a plurality (several to several tens) of monosaccharide units selected from glucose, mannose, N-acetylglucosamine, frucose, galactose, glucuronic acid, N-acetylglucosamine, and sialic acid. Also, in the method of the invention, there is included a glycoprotein, which is a composite of such a sugar chain and a protein, a glycolipid, which is a composite with a lipid, or, in the case where such a sugar chain is expressed at the surface of a living cell, a cell itself expressing the sugar chain at the surface of the cell membrane or a fragment of the cell membrane.

“Lipid” includes a composite lipid, a natural lipid (acylglycerol), a lipoprotein, which a composite with a protein, a phospholipid, such as lecithin, locating in a tissue boundary lipid membrane, such as a cell organelle membrane, such as a neural tissue, a plasma membrane, mitochondria, a microsome, or a cell nucleus, and a liposome as a double-membrane lipid capsule.

The core material to be employed in the invention may have a particulate shape (form), a flat shape or a film shape, but preferably is in a particulate form at a consideration for a specific separating method by a magnetic operation, and is more preferably constituted of fine particles of a particle size of 0.001 to 10 μm in consideration of dispersion into liquid.

In the case of employing a method of immobilizing the PHA synthetase principally by the ion adsorption method, there can be employed a core having an ionic functional group on the surface, for example, a clay mineral, such as caolinite, bentonite, talc or mica, a metal oxide, such as alumina or titanium dioxide, or an insoluble inorganic salt, such as silica gel, hydroxy apatite, or calcium phosphate gel. Also, a polymer having an ionic functional group, such as an inorganic pigment, an ion exchange resin, a chitosan, or a polyaminopolystyrene including these materials as a principal component, can also be utilized as an ion adsorbing core.

In the case where the base material is constituted of a magnetic material, for example, a metal oxide, such as ferrite, a hydroxyl group is present on the surface thereof and can be advantageously utilized for fixing via a hydrogen bonding with the carboxy group on the surface of the PHA synthetase.

On the other hand, in the case of immobilizing the PHA synthetase principally by hydrophobic adsorption, there can be utilized a core with a non-polar surface, for example various polymers lacking an ionic functional group on the surface or showing a hydrophobic group on the surface, such as a styrenic polymer, an acrylic polymer, a methacrylic polymer, a vinyl ester or a vinylic polymer. More specifically, an organic pigment, such as an azo pigment having plural aromatic rings, a phthalocyanine pigment or an anthraquinone pigment of a condensed polycyclic structure, or carbon black, has a hydrophobic adsorbing property. The hydrophobic adsorption method is also applicable to a magnetic material subjected to an oleophilic treatment.

The fixing of the PHA synthetase to a core by an ionic adsorption method or a hydrophobic adsorption method can be achieved by mixing the PHA synthetase and the core in a predetermined reaction liquid. In this operation, it is preferable to shake or agitate a reaction vessel with a suitable intensity in order for the PHA synthetase to be uniformly adsorbed on the surface of the core.

As the polarity and amount of the surface charge and the hydrophobicity on the core and the PHA synthetase vary depending on the pH, salt concentration and temperature of the reaction liquid, it is desirable to regulate the solution within a permissible range, according to the nature of the core to be employed. For example, in the case where the core principally shows an ionic adsorption property, it is possible to increase the charge amount, contributing to the adsorption between the core and the PHA synthetase by reducing the salt concentration. It is also possible to increase opposite charges on both components by a change in the pH. Also, in the case where the core principally shows a hydrophobic adsorption property, the hydrophobicity of both components can be increased by an increase in the salt concentration. It is also possible to investigate the charge state or the hydrophobicity of the core and the PHA synthetase by an electrophoretic measurement or a wetting angle measurement in advance, and to select a solution condition suitable for the adsorption. Furthermore, such a condition can be determined by a direct measurement of the adsorbed amount of the core and the PHA synthetase. The adsorption amount can be measured, for example, by a method of adding a solution of the PHA synthetase of a known concentration to a core dispersion, then, after an adsorption process, measuring the concentration of the PHA synthetase in the solution and determining the amount of the adsorbed enzyme by a subtraction.

In the case of a core on which the enzyme is difficult to fix by the ionic adsorption or the hydrophobic adsorption, a covalent bonding method may be adopted if the operation is not too cumbersome and a possibility of enzyme deactivation can be tolerated. There can be employed, for example, a method of executing a diazo formation on a core material (solid particle) having an aromatic amino group, and executing a diazo coupling of the enzyme thereto, a method of forming a peptide bond between the core material (solid particle) having a carboxy group or an amino group and the PHA synthetase, a method of alkylation between the core material (solid particle) having a halogen group (halogenoalkyl group) and an amino group of the PHA synthetase, a method of reacting a polysaccharide core particle activated with cyan bromide with an amino group of the PHA synthetase, a method of cross-linking an amino group of the core material (solid particle) and an amino group of the enzyme, a method of reacting the core material (solid particle) having a carboxy group or an amino group with the PHA synthetase in the presence of a compound having an aldehyde group or a ketone group and an isocyanide compound, or a method of executing an exchange reaction between the core material (solid particle) having a disulfide group (—S—S—) and a sulfanyl group (—SH) of the PHA synthetase.

It is also possible to adsorb the PHA synthetase on the surface of the core material (solid particle) by an affinity adsorption. The affinity adsorption is a biological adsorption between a biological polymer and a specified substance called a ligand and showing a specific affinity thereto. It can take place, for example, between an enzyme and a substrate, an antibody and an antigen, a receptor and an information material, such as acetylcholine, or mRNA and tRNA. In the case of immobilizing an enzyme protein by affinity adsorption, there is generally employed a method of binding a substrate or a reaction product of the enzyme, a competitive inhibitor, a coenzyme or an allosteric effector as a ligand to a solid surface and realizing an affinity adsorption on such a solid surface through a binding between such a ligand and an added enzyme protein. However, in the PHA synthetase, in the case where the 3-hydroxyl CoA constituting the substrate therefor is employed as the ligand, an active position of the PHA synthetase, catalyzing the PHA synthesis, is blocked by the binding with the ligand, thus becoming unable to synthesize the PHA. Nevertheless, the PHA synthesizing activity of the PHA synthetase can be maintained even after the fixing by affinity adsorption, by fusing another biological polymer to the PHA synthetase in advance and executing affinity adsorption utilizing a ligand for such a biological polymer. The fusion of the PHA synthetase and the biological polymer may be achieved by a genetic engineering method, or by chemically binding the biological polymer to the PHA synthetase. There may be utilized any biological polymer as long as a ligand thereto is easily available and can be easily coupled to the core, but, in case a fused substance is developed by a gene recombination, the biological polymer to be fused is preferably a protein. More specifically, Escherichia coli in which a fusion gene of the GST gene and a gene sequence of the PHA synthetase are introduced by transformation is utilized for producing a fused protein of the GST and the PHA synthetase, and such a fused protein is added to sepharose coupled with glutathione serving as a ligand for the GST, thereby enabling affinity adsorption of the PHA synthetase of fused protein type on the sepharose.

As already described, it is furthermore possible to fuse a peptide, including an amino acid sequence having a binding ability to the magnetic material, with the PHA synthetase, and to fix the PHA synthetase to the surface of such a magnetic material based on a binding property between the peptide portion of the amino acid sequence having the binding ability to the magnetic material and the magnetic material.

Immobilization of Target Component-Binding Molecule on PHA Construct

For immobilizing a molecule having a binding affinity to the target component (hereinafter represented as a target component-binding molecule) on the surface of the PHA magnetic construct, there can be utilized a physical adsorption by a physical affinity, such as hydrophobicity, an ionic or van der Waals force between the PHA coating the surface and the target component-binding molecule, but, in consideration of reproducibility and stability, it is more desirable to form an irreversible covalent bond by combining a functional group in the side chain of the PHA and a functional group present in the target component-binding molecule either directly or in the presence of a converting/modifying/activating reagent.

As a configuration of the PHA construct to be employed in the method of the invention, there can be employed a PHA construct having an epoxy group on the side chain of the PHA coating the surface. Such an epoxy group can form a covalent bond directly with an amino group (—NH₂) or a sulfanyl group (—SH) provided in the target component-binding molecule. Since the covalent bond can be formed without requiring an agent, this method is useful for immobilizing an easily denatured protein, such as an enzyme protein, or an antibody (Fc) receptor protein, such as an A protein or a G protein. It is also possible to achieve an efficient recovery of a target protein with a histag, by immobilizing iminodiacetic acid (IDA) on the surface coating the PHA and adding a metal ion such as Ni²⁺. This method of forming a covalent bond utilizing the reaction with the epoxy group is also applicable if the target component-binding molecule is a low molecular chemical substance, such as a drug candidate, as long as such a low molecular chemical substance has an amino group (—NH₂) or a sulfanyl group (—SH).

Such an epoxy-containing PHA construct can be converted into a PHA construct having an amino group by a reaction under alkaline conditions with ammonium hydroxide or hexamethylene diamine hydrochlorate salt at 10 to 100 molar amount with respect to the epoxy group. If the target component-binding molecule is a protein or a peptide, such an amino group can form by means of a cross-linking agent, such as NHS (N-hydroxysuccinimide), with a terminal carboxy group in the main chain thereof or a carboxy group of a side chain of a residue of an amino group, such as aspartic acid or glutamic acid present in such a protein or peptide. In such a case, it is necessary to convert the carboxy group of the target component-binding molecule into an activated ester by an NHS treatment, and it is necessary to confirm, in advance, whether the function of the target component-binding molecule is sufficiently sustained after the NHS treatment. This immobilization method utilizing the amide bond formation is also applicable when the target component-binding molecule is a low molecular chemical substance, such as a drug candidate, as long as such a low molecular chemical substance has a carboxy group.

Furthermore, if the target component-binding molecule is a glycoprotein, such as a sugar chain or lectin, a glycolipid, such as a lipopolysaccharide, the amino group can form a stable bond with an aldehyde structure (formyl group: —CHO) in the sugar chain by a Schiff's base (—CH═N—) formation and a reductive amination. This covalent bond forming method utilizing a reaction between the sugar chain and the amino group is accelerated by a partial oxidation of the sugar chain portion, introducing an aldehyde structure in the sugar chain, and also is applicable for immobilizing an antibody molecule, such as IgG, having a sugar chain in the Fc portion thereof. This method utilizing the amino group and the aldehyde structure (formyl group: —CHO) is also applicable when the target component-binding molecule is a low molecular chemical substance, such as a drug candidate, as long as such a low molecular chemical substance has an aldehyde structure (formyl group: —CHO) or can introduce an aldehyde structure (formyl group: —CHO) by a partial oxidation.

Furthermore, such an amino group can be coupled with a target component-binding molecule having a sulfanyl group (—SH) in the presence of a maleimide derivative, a pyridylthio compound or an iodine/bromine acetyl compound. Basic reacting conditions of the amino group and the sulfanyl group (—SH) are, in case of employing a maleimide derivative, 2 to 4 hours at 4° C. to room temperature in 0.1 M sodium phosphate (pH 6.5 to 7.5) Also, in the case of employing a pyridylthio compound, 15 to 20 hours at the room temperature in a PBS buffer (pH 7.5), and, in the case of employing an iodine/bromine acetyl compound, 1 hour at the room temperature and in the absence of light, in a 0.5 M sodium borate solution (pH 8.3), but such reaction conditions may be suitably changed according to the type of the target component-binding molecule and an application thereafter.

Another configuration of immobilizing on the PHA construct to be employed in the method of the invention utilizes a PHA construct having a carboxy group on the side chain of the PHA. If the target component-binding molecule is a protein or a peptide, such a carboxy group can form an amide bond by means of a cross-linking agent, such as NHS (N-hydroxysuccinimide), with a terminal amino group of the main chain thereof or an amino group on a side chain of a residue of an amino group, such as lysine or arginine, present in the protein or peptide. This amide forming reaction can be improved in rate and frequency by converting the carboxy group of the PHA into an activated ester in advance. Also, in this amide forming method utilizing the carboxy group of the PHA side chain, if the target component-binding molecule is a DNA or an oligonucleotide, it can be carried on the PHA by the afore-described reaction by employing DNA or oligonucleotide of which a terminal is converted into an amino group by an already known method. This amide forming method utilizing the carboxy group of the PHA side chain is also applicable when the target component-binding molecule is a low molecular chemical substance, such as a drug candidate, as long as such a low molecular chemical substance has an amino group.

Another configuration of immobilizing on the PHA construct to be employed in the method of the invention utilizes a PHA construct having a halogen, such as a chloro group (—Cl), a bromo group (—Br) or a fluoro group (—F), on the side chain of PHA. Such a halogen, if it is a chloro group or a bromo group, can form a sulfide bond (—S—) with the target component-binding molecule having a sulfanyl group (—SH) under mild conditions.

It is furthermore possible to bind a substance showing a specific binding to the “target component-binding molecule” (for example such a substance being protein A or protein G when the “target component-binding molecule” is an antibody) to be immobilized on the construct coated on the surface with the PHA, to such a surface utilizing the afore-described active functional group, and then to cause a specific binding of the desired “target component-binding molecule” to such a substance showing specific binding with the “target component-binding molecule”, thereby achieving immobilization thereof on the construct. Also, it is possible to immobilize the “target component-binding molecule” after a further modification. As an example of the latter, biotin is immobilized on a carboxy type PHA construct utilizing a reagent NHS-iminobiotin (manufactured by Pierce Inc.), and a target component-binding molecule modified with avidin or streptoavidin is carried on the construct by a specific binding with biotin. Examples of usable binding pair include lectin and sugar, haptene and antibody, protein A or protein G and antibody Fc, other protein pairs showing specific binding, phenylboronic acid and salicylhydroxamic acid, and other pairs of chemical portions, which mutually react but do not react with a protein.

In order to prevent a non-specific adsorption on the construct, it is preferred to coat a non-carrying portion of the surface of the construct with a “blocking agent”, which does not deteriorate the activity of the carried target component-binding molecule. Examples of the block agent suitable for such “blocking treatment” include collagen, gelatin (particularly cold-water fish hide gelatin), skimmed milk, a serum protein, such as BSA, and various compounds including a hydrophobic portion and a hydrophilic portion, which do not react with protein.

Contact and Binding Between PHA Construct Carrying Target Component-Binding Molecule and Target Component

In the method of the invention, contact between a PHA construct carrying a target component-binding molecule and a target component is usually carried out in an aqueous medium, but, if the target component has a low solubility in water as in certain drug candidates, the contact may be carried out in an emulsion system by adding a polar solvent, such as an alcohol, acetone, DMSO (dimethyl sulfoxide) or DMF (dimethylformamide) with a surfactant such as Tween, Triton or SDS, and eventually a non-polar solvent, such as toluene, xylene or hexane, thereby accelerating the binding reaction. However, if a solvent or surfactant is employed, a concentration thereof has to be selected within such a range that does not deteriorate the affinity binding function of the carried target component-binding molecule.

In the method of the invention, in order to stimulate the contact and binding between the PHA construct carrying the target component-binding molecule and the target component, a heating means or an agitating means may be employed within an extent not deteriorating the affinity binding function of the target component-binding molecule, and an ultrasonic means may also be employed.

When the construct to be employed in the method of the invention is a magnetic construct, a manipulation utilizing a magnetic force is also possible for stimulating contact and binding between the target component-binding molecule carried on the surface of the construct and the target component. In a manipulation by the magnetic force, it is possible to repeat the application and release of the magnetic force, utilizing a structured member for generating a magnetic force (such as a permanent magnet or an electromagnet, which may hereinafter be collectively called a magnet). In case of employing an electromagnet, a current supply and a current cut-off are repeated by a switching operation, thereby repeating the capture and release of the magnetic construct. As an example of the operation utilizing a magnet, a probe-shaped electromagnet is inserted into a reaction vessel, and a current supply and a current cut-off to the magnetic are repeated by a switching operation or an on-off operation of a power supply, thereby repeating the capture and release of the magnetic construct. In another example, a magnet is positioned outside a reaction vessel, and an applied intensity of the magnetic field is repeatedly varied by a switching operation, an on-off operation of a power supply, or a regulation of the distance of the magnet to the reaction vessel, thereby repeating the capture and release of the magnetic construct.

In the method of the invention, “binding” between the target component and the target component-binding molecule means a specific binding of a molecule to the other by a chemical or physical action between the pair. Examples of such binding include not only a binding between an antigen and an antibody by a known antigen-antibody reaction, but also binding between biotin and avidin, between a hydrocarbon and lectin, between complementary sequences of nucleic acid and nucleotide, between molecules of an actuator and a receptor, between a co-enzyme and an enzyme, between an enzyme inhibitor and an enzyme, between a peptide sequence and an antibody specific to such a sequence or to all the proteins, between an acid and a base in a polymer, between a dye and a protein binder, between a peptide and a specific protein binder (ribonuclease, S-peptide and ribonuclease S-protein), between a sugar and boric acid, and between similar molecule pairs having an affinity enabling a molecular association in a binding assay, but such an examples are not restrictive. Also, the binding pair may be elements similar to original binding elements, such as analogs of the substance to be analyzed or binding elements, produced by recombination or molecular engineering. In case the binding elements are immuno-reactive, there can be employed an antibody, an antigen, a haptene or a complex thereof, and, in case of employing an “antibody,” there can be utilized a monoclonal antibody, a polyclonal antibody, a recombinant antibody, a natural antibody, a chimera antibody, a mixture(s), a single-chain antibody-displaying phage (including the entire phage), a fragment(s) thereof expressing the single-chain antibody, or a mixture of binding elements of antibody and protein.

Along with the recent progress in the evolution molecular engineering, there has been developed a technology of screening a nucleic acid molecule, or an aptamer (also called a nucleic acid antibody), having a high affinity to a target molecule, such as protein from a random oligonucleotide library (“systematic evolution of ligands by exponential enrichment”; SELEX or in vitro selection). This screening method based on the aptamer has been applied to the preparation of high affinity ligands easier and faster than antibodies (for example, Nature, 355:564 (1992), International Patent Application No. WO 92/14843, Japanese Patent Application Laid-Open Nos. H08-252100 and H09-216895).

Also, binding between a transcription factor being a protein and a nucleic acid having a specified base sequence is expected in clarifying causes of diseases and in applications for effective diagnosis and therapy.

The “binding” considered in the method of the invention naturally includes such nucleic acid-protein affinity binding.

Also, the “binding” considered in the method of the invention includes any and all physical or chemical adhesion, and specific/selective association, regardless whether it is permanent or temporary. In general, it is possible to cause a physical adhesion between a ligand molecule and a receptor by an ionic interaction, a hydrogen bonding, a hydrophobic force or a van der Waals force. The interaction of “binding” may be short in time, as in the case where a chemical change is induced by the binding. This generally applies to a case where the binding component is an enzyme and the “target component-binding molecule” is a substrate for the enzyme. Also, the chemical connection may be irreversible or reversible. The binding may also become specific under a particularly different condition.

In the method of the invention, a practical example of the contacting and binding process between the target component-binding molecule carried on the PHA magnetic construct and the target component is a process of contacting a PHA magnetic construct carrying a target component-binding molecule with a biological specimen containing a natural protein, such as a tissue, a cell homogenate or a fluid, such as serum, thereby causing specific adsorption and binding between such a natural protein with the target component-binding molecule carried on the surface of the PHA magnetic construct.

Another example of such a process employs a construct carrying a protein constituting the target component coupling molecule, thereby causing a selective coupling with an antibody portion displayed on the surface of a suitable bacteriophage, as in the known phage display antibody selecting method.

Also, as another example, there can be conceived a process of contacting with a biological specimen containing a receptor, for example, a liquid, such as a hybridoma supernatant or a phage display fluid, thereby causing the receptor contained in such a biological specimen to be specifically adsorbed on the target component-binding molecule carried on the PHA magnetic construct.

Magnetic Separation and Washing of Magnetic Composite Binding Target Component

In a method of binding the target component in a magnetic composite (magnetic construct in which the target component-binding molecule is carried) and separating and washing the magnetic composite binding the target component, a magnetic separating operation is carried out by repeating exertion and release of the magnetic force with a structured member generating a magnetic force (such as a permanent magnet or an electromagnet). In case of employing an electromagnet, power supply and cut-off to the electromagnet are carried out by a switch operation to capture and release the magnetic composite binding the target component.

In an embodiment, a probe-shaped magnet is used to capture the magnetic composite binding the target component in a container, and remaining liquid is removed from the container. Then, a washing liquid is charged into the container to wash the magnetic composite binding the target component still captured on the probe. Otherwise, the washing operation can be carried out by taking the probe-shaped magnet, on which the magnetic composite binding the target component is captured, from a reaction liquid and moving it to a washing liquid.

In another method, a magnet is positioned outside a container to attract the magnetic composite binding the target component to an internal wall of the container at a replacement of the liquid. By removing such an external magnet, the magnetic composite binding the target component is released and is mixed with the liquid (washing liquid), whereby a washing operation can be carried out.

For such a magnetic separating operation, there can be utilized various magnetic selecting equipment commercially available for the purpose of manipulating magnetic particles. Examples of a magnetic selecting equipment include DYNALMCP, manufactured by DYNAL Inc., MAIA Magnetic Separator, manufactured by Serono Diagnostics Inc., Magnetight™ Separation Stand, manufactured by Takara Shuzo Co., and BioMag Separator, manufactured by Advanced Magnetics Inc.

Elution and Liberation of Target Component from PHA Construct

In the method of the invention, it is also possible, after isolation of the magnetic composite binding the target component, to elute/liberate the binding component from the target component-binding molecule, if necessary. If the target component-binding molecule and the target component are a protein-protein combination, they can be liberated under an ordinary liberating condition (pH 2, 4M guanidine, 2M ammonium thiocyanate, 1% SDS etc.), but the liberated target component protein is often denatured. The liberated target component protein, even if denatured, is acceptable if it is purified by electrophoresis or HLPC, but a restoring process, such as a dialysis, may be required if an original steric structure, etc., of the target component protein is to be confirmed.

Detection of Target Component

In the method of the invention, the detection of the target component may be carried out by any method employable in an immunoassay or a hybridization assay, such as an ordinary colorimetry, a fluorescent method, a chemiluminescence method or a radioisotope method. Also, the target component eluted/liberated by the afore-described method from the target component-binding molecule can be analyzed by similar methods. It is also possible, if the target component is DNA, to verify the base sequence by a sequencer after an amplification by PCR or the like, and, if the target component is a protein, to carry out an enzymatic decomposition, then to separate the enzyme digested fragments by two-dimensional electrophoresis or HPLC and to carry out an electrospray/ionization and a mass spectroscopic analysis, or, to analyze the target component protein either directly or after enzymatic decomposition, by a matrix assisted laser deionization-time of flight mass spectroscopy (MALDIToF-MS).

It is naturally possible also to detect the target component by nuclear magnetic resonance (NMR) spectroscopy, infrared absorption (IR) spectroscopy or ultraviolet absorption (UV) spectroscopy, used either singly or in combination.

### EXAMPLES

The present invention will be explained in more detail in the following Examples. These examples represent the optimum embodiments of the present invention, but the present invention is by no means limited thereto. The following Examples employ a polyhydroxyalkanoate including a 3-hydroxyalkanoic acid unit, but any polyhydroxyalkanoate can be utilized. In the following, “%” refers to mass percent, unless otherwise specified, and the term “part” refers to a part by mass.

First, a method for preparing a magnetic material as a common base material will be shown.

### Reference Example 1

### Preparation of Magnetic Material

In an aqueous solution of ferrous sulfate, a solution of sodium hydroxide of 1.0 to 1.1 equivalents to iron ions was added to prepare an aqueous solution containing ferrous hydroxide. Then, air was blown in while the solution was maintained at a pH of about 8 to carry out an oxidation reaction at 80 to 90° C. to prepare a slurry for generating seed crystals.

Then, to this slurry, an aqueous solution of ferrous sulfate was added in an amount of 0.9 to 1.2 equivalents with respect to the initial alkali amount (sodium component of sodium hydroxide), and the oxidation reaction was carried out by blowing air while the slurry was maintained at a pH of about 8. Magnetic iron oxide particles generated after the oxidation reaction were washed, filtered and dried, and the coagulated particles were broken to obtain a magnetic material (1) of an average particle size of 0.10 μm.

In the following, there is shown an embodiment on scl-PHA synthetase (Reference Examples 2 to 4, Examples 1 to 10).

### Reference Example 2

### Preparation of Transformant Having scl-Pha Synthetase Producing Ability

Strain TB64 was cultured in 100 ml of LB medium (1% polypeptone, 0.5% yeast extract, 0.5% sodium chloride, pH 7.4) overnight at 30° C. Then, the chromosomal DNA was isolated by the method of Marmar et al. The obtained chromosomal DNA was partially digested by a restriction enzyme Sau3Al. A vector pUC18 was also cut by a restriction enzyme BamHI. After terminal dephosphorylation (Molecular Cloning, 1, 572, (1989); Cold Spring Harbor Laboratory Press), Sau3AI partial digestion fragments of the chromosomal DNA were ligated to the cleavage site of the vector using a DNA ligation kit Ver. II (TAKARA SHUZO CO., LTD.). With these ligated chromosomal DNA fragments, Escherichia coli HB 101 was transformed to construct a chromosomal DNA library of strain TB64.

Next, to obtain DNA fragments covering the PHB synthetase gene of strain TB64, an expression screening was carried out. LB culture medium containing 2% glucose was used for screening, and a Sudan black solution was sprayed when the colony grown on the agar medium plate to a suitable size, and a colony showing fluorescence under UV light irradiation was collected. The DNA fragment covering the PHB synthetase gene was obtained by recovering the plasmid from the acquired colony with the alkali method.

The acquired gene fragment was transformed into a vector pBBR122 (Mo Bi Tec) having a wide-host-replication range (Mo Bi Tec) not belonging to any of IncP, IncQ, or IncW incompatibility group. When Ralstogna eutropha TB64 ml strain (PHB synthesis negative strain) was transformed with this recombinant plasmid by electroporation, the PHB synthesizing capacity of the TB64 m1 strain was recovered to show complementarity.

The base sequence was determined by Sanger method on the fragment covering the PHB synthetase gene. As a result it was confirmed that the PHB synthetase gene indicated by SEQ ID NO: 1 was present in the fragment.

Then, an oligonucleotide having the base sequence in the vicinity of the starting codon of the scl-PHA synthetase gene was designed and synthesized (Amersham Pharmacia Biotech), and PCR was carried out by using the oligonucleotide as a primer to amplify the fragment, including the scl-PHA synthetase gene (LA-PCR kit; TAKAPA SHUZO).

The obtained PCR-amplified fragment was completely digested by a restriction enzyme BamHI. A vector pTrc99A was also cut by BamHI. After terminal dephosphorylation (Molecular Cloning, 1, 572, (1989); Cold Spring Harbor Laboratory Press), complete BamHI digestion fragments were ligated using a DNA ligation kit Ver. II (TAKARA SHUZO CO., LTD.).

With the obtained recombinant plasmid vectors, Escherichia coli HB 101 was transformed by the calcium chloride method (TAKARA SHUZO) to recover a recombinant plasmid pTB 64-phb from the transformant.

Then, with pTB 64-phb, Escherichia coli HB 101 was transformed by the calcium chloride method to obtain a pTB 64-phb transformant strain.

### Reference Example 3

Production of scl-PHB synthetase (1)

Construction of Transformant Having GST Fused scl-PHA

### Synthetase Production Capacity

The pTB 64-phb transformant strain was inoculated in 200 ml of an LB medium and incubated at 37° C. with shaking at 125 strokes/min. After being cultured for 12 hours, 200 ml of the culture liquid was inoculated in 200 ml of an LB medium (total 400 ml) and incubated for 12 hours at 37° C. with shaking at 125 strokes/min. The cells were harvested by centrifugation and plasmid DNA was recovered by the normal method.

Then, an oligonucleotide (SEQ ID NO: 3) constituting an upstream primer to the pTB64-phb and an oligonucleotide (SEQ ID NO: 4) constituting a downstream primer were designed and synthesized respectively (Amersham Pharmacia Biotech). Using these oligonucleotides as primers, PCR was carried out by using pTB 64-PHB as a template to amplify a full length of scl-PHA synthetase gene having a BamHI cleavage site upstream and an Xhol cleavage site downstream (LA-PCR kit; TAKARA SHUZO CO., LTD.).

The purified PCR amplification product was digested by BamHI and XhoI, then inserted into the corresponding restriction sites of plasmid pGEX-6P-1 (Amersham Pharmacia Biotech). An E. coli strain (JM109) was transformed with this vector, and consequently, a strain for the expression was obtained. For confirmation, a large amount of the plasmid DNA was prepared using Miniprep (Wizard Minipreps DNA Purification Systems, PROMEGA) and digested by BamHI and XhoI, and the resulting DNA fragment was identified.

Preparation of scl-PHA Synthetase

The obtained expression strain was pre-cultured overnight at 30° C. in 100 mL of 2×YT culture medium (polypeptone 16 g/L, yeast extract 10 g/L, NaCl 5 g/L, pH 7.0) with added ampicillin (100 μg/L). Then, it was added to 10 liters of 2×YT culture medium with added ampicillin (100 μg/L) and culture was carried out for 3 hours at 30° C. Then, isopropyl-β-D-thiogalactopyranocide (IPTG) was added to obtain a final concentration of 1 mmol/L, and the culture was continued for 3 hours at 30° C.

The recovered culture liquid was centrifuged for 10 minutes at 4° C., 78,000 m/s² (=8,000 G), and, after the elimination of the supernatant, the pellet was re-suspended in 500 mL of a PBS solution at 4° C. The cell suspension was poured, 40 ml each time, in a vessel cooled to 4° C. in advance, and, under the pressure of 216 MPa (=2,200 kg/cm2) applied by a French press, the cell suspension was released little by little from the nozzle to disrupt the cells. The cell lysate was centrifuged for 10 minutes at 4° C., 78,000 m/s² (=8,000 G), and the supernatant was recovered. The liquid was filtered with a filter of 0.45 μm to eliminate the solids. The presence of the desired scl-PHA synthetase fused to glutathione transferase (GST) in the supernatant was confirmed by SDS-PAGE.

Then, the GST-fused PHB synthesizing enzyme was purified with glutathione sepharose 4B (Amersham Pharmacia Biotech Inc.). 6.65 ml of 75% slurry of glutathione sepharose 4B was centrifuged for 5 minutes at 4° C., 4,900 m/s² (=500 G), and, after the elimination of supernatant, it was re-suspended in 200 ml of a PBS solution at 4° C. Centrifugation was carried out again for 5 minutes at 4° C., 4,900 m/s² (=500 G), and the supernatant was removed. Then, it was re-suspended in 5 ml of a PBS solution at 4° C. to obtain 50% slurry of glutathione sepharose 4B.

The entire amount of the supernatants prepared before was added to 10 ml of thus obtained 50% slurry of glutathione sepharose 4B, and the mixture was mildly shaken for 30 minutes at the room temperature to cause affinity adsorption of the desired fused protein in the supernatant to the glutathione sepharose 4B. The liquid was centrifuged for 5 minutes at 4° C., 4,900 m/s² (=500 G), and, after the elimination of the supernatant, it was re-suspended in 5 ml of a PBS solution at 4° C., and subjected to similar centrifugation again and the supernatant was removed. The glutathione sepharose 4B on which GST-fused scl-PHA synthetase was immobilized was taken as an immobilized enzyme (1).

Then, after rinsing by repeating the re-suspension in a PBS solution and centrifuging twice, it was finally suspended in 5 ml of Cleavage buffer (Tris-HCl 50 mmol/L, NaCl 150 mmol/L, EDTA 1 mmol/L, dithiothreitol 1 mmol/L, pH 7). Then, 0.5 ml of 4% solution of PreScission Protease (Amersham Pharmacia Biotech) in the cleavage buffer were added, and the mixture was mildly shaken for 4 hours at 5° C. The mixture was centrifuged for 5 minutes at 4° C., 4,900 m/s² (=500 G), and the supernatant was recovered. Then, 1 ml of 50% slurry of glutathione sepharose 4B prepared as explained in the foregoing was centrifuged for 5 minutes at 4° C., 4,900 m/s² (=500 G), to which the above-recovered supernatant was added, and the mixture was mildly agitated to cause glutathione sepharose 4B to adsorb PreScission Protease remaining in the supernatant. Then, centrifugation was carried out for 5 minutes at 4° C., 4,900 m/s² (=500 G), and the supernatant was recovered. The supernatant showed a single band in SDS-PAGE, indicating the purification.

The activity of the contained scl-PHA synthetase was measured in the following manner. At first, bovine serum albumin (Sigma Co.) was dissolved in 0.1 mol/L tris-HCl buffer (pH 8.0) in 3.0 mg/ml, and 100 μl of thus obtained solution was added to 100 μl of the enzyme solution and the mixture was pre-incubated for 1 minute at 30° C. Then, 100 μl of solution of 3-hydroxybutyryl CoA dissolved in 0.1 mol/L tris-HCl buffer (pH 8.0) in 3.0 mmol/L was added. Then, the mixture was incubated for 1 to 30 minutes at 30° C., and the reaction was terminated by adding a solution of trichloroacetic acid dissolved in 0.1 mol/L tris-HCl buffer (pH 8.0) at 10 mg/ml. The solution, after the termination of the reaction, was centrifuged (147,000 m/s² (15,000 G), 10 minutes), and 500 μl of 2.0 mmol/L solution of 5,5′-dithiobis-(2-nitrobenzoic acid) dissolved in 0.1 mol/L tris-HCl buffer (pH 8.0) was added to 500 μL of the supernatant and, after incubation for 10 minutes at 30° C., the optical absorbance at 412 nm was measured. The enzyme activity was calculated by taking an enzyme amount causing release of CoA of 1 μmol in 1 minute as 1 unit (U). As a result there was obtained a specific activity of 7.5 U/ml. The obtained liquid was concentrated by ultra-filtration under the addition of a lyphogel to 10 U/mL, thereby obtaining a purified enzyme liquid (1).

### Reference Example 4

### Preparation of Crude Enzyme Liquid Containing Scl-Pha Synthetase

The KK01 and TL2 strains were cultured for 24 hours at 30° C. in 100 liters of the M9 culture medium containing 0.5% of yeast extract and 0.3% of mineral solution (see below), and the recovered culture liquid was centrifuged for 10 minutes at 4° C., 78,000 M/s² (=8,000 G), and, after the elimination of the supernatant, the cell pellet was re-suspended in 500 ml of a PBS solution at 4° C. The cell suspension was poured, 40 ml each time, in a vessel cooled to 4° C. in advance, and, under the pressure of 216 MPa (=2,200 kg/cm²) applied by a French press, the cell suspension was released little by little from the nozzle to disrupt cells. The disrupted cell suspension was centrifuged for 10 minutes at 4° C., 78,000 M/s² (=8,000 G), and the supernatant was recovered. The liquid was filtered with a filter of 0.45 μm to eliminate the solids, and the activity of the contained scl-PHA synthetase was measured by the afore-described method. As a result, there were obtained relative activities of 1.6 U/mL for the KK01 strain and 1.2 U/mL for the TL2 strain. The liquid was concentrated by ultra-filtration under the addition of a lyphogel to 10 U/mL, thereby obtaining crude enzyme liquid (1) derived from the KK01 strain and crude enzyme liquid (2) derived from the TL2 strain.

### Example 1

The purified scl-PHA synthetase liquid was used to prepare a magnetic capsule construct (1) in the following manner.

To 10 parts by mass of the purified enzyme liquid (1), 1 part by mass of the magnetic material (1) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described enzyme-immobilized magnetic material was suspended in 48 parts by mass of 0.1 mol/L phosphate buffer (pH 7.0), and then, 1 part by mass of 3-hydroxybutyryl CoA (Sigma Aldrich Japan Co.) and 0.1 part by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with PHB on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried under vacuum conditions, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=68,000 and Mw=140,000.

### Example 2

The crude enzyme liquid containing scl-PHA synthetase was used to prepare a magnetic capsule construct (2) in the following manner.

To 10 parts by mass of the crude enzyme liquid (1) 1 part by mass of the magnetic material (1) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described enzyme-immobilized magnetic material was suspended in 48 parts by mass of 0.1 mol/L phosphate buffer (pH 7.0), and then, 1 part by mass of 3-hydroxybutyryl CoA (Sigma Aldrich Japan Co.) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried under vacuum conditions, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=59,000 and Mw=130,000.

### Example 3

The crude enzyme liquid containing scl-PHA synthetase was used to prepare a magnetic capsule construct (3) in the following manner.

To 10 parts by mass of the crude enzyme liquid (2) 1 part by mass of the magnetic material (1) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of 3-hydroxybutyryl CoA (Sigma Aldrich Japan Co.) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried in a vacuum, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=65,000 and Mw=160,000.

### Example 4

The purified scl-PHA synthetase liquid was used to prepare a magnetic capsule construct (4) in the following manner.

To 10 parts by mass of the purified enzyme liquid (1), 1 part by mass of nickel powder of a primary particle size of 0.02 μm (Ni(200)UFMP, Shinku Yakin Co.) as the magnetic material (2) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of 3-hydroxybutyryl CoA (Sigma Aldrich Japan Co.) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of nickel powder of a primary particle size of 0.02 μm (Ni(200)UFMP, Shinku Yakin Co.) as the magnetic material (2) was added to 49 parts by mass of 0.1 mol/L phosphate buffer (pH 7.0), then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried in a vacuum, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=78,000 and Mw=170,000.

### Example 5

The purified scl-PHA synthetase liquid was used to prepare a magnetic capsule construct (5) in the following manner.

To 10 parts by mass of the purified enzyme liquid (1), 1 part by mass of γ-Fe₂O₃ fine powder of a primary particle size of 0.02 μm (NanoTel, CI Chemical Co.) as the magnetic material (3) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of 3-hydroxybutyryl CoA (Sigma Aldrich Japan Co.) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of γ-Fe₂O₃ fine powder of a primary particle size of 0.02 μm (NanoTek, CI Chemical Co.) as the magnetic material (3) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 M/s² (=10,000 G), 4° C., 10 minutes), dried in a vacuum, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=80,000 and Mw=170,000.

### Example 6

The purified scl-PHA synthetase liquid was used to prepare a magnetic capsule construct (6) in the following manner.

To 10 parts by mass of the purified enzyme liquid (1), 1 part by mass of magnetite fine powder of a primary particle size of 0.3 μm (Magnetite EPT500, Toda Kogyo Co.) as the magnetic material (4) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of 3-hydroxybutyryl CoA (Sigma Aldrich Japan Co.) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of magnetite fine powder of a primary particle size of 0.3 μm (Magnetite EPT500, Toda Kogyo Co.) as the magnetic material (4) was added to 49 parts by mass of 0.1 mol/L phosphate buffer (pH 7.0), then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried in a vacuum, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=79,000 and Mw=180,000.

### Example 7

The purified scl-PHA synthetase liquid was used to prepare a magnetic capsule construct (7) in the following manner.

To 10 parts by mass of the purified enzyme liquid (1), 1 part by mass of the magnetic material (1) and 39 parts by mass of PBS were added and mildly shaken for 30 minutes at 30° C. to cause the scl-PHA synthetase to be adsorbed on the surface of the magnetic material. The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), then 1 part by mass of 3-hydroxybutyryl CoA (Sigma Aldrich Japan Co.), 1 part by mass of polyethylene glycol 200 (PEG200, Kishida Kagaku Co., average molecular weight 190 to 210) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material was observed to confirm that the magnetic material was coated with the PHB on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material did not show fluorescence at all.

Also, a part of the synthesized PHB particles was collected by centrifugation (98,000 m/s² (=10,000 G), 4° C., 10 minutes), dried in a vacuum, suspended in chloroform and agitated for 20 hours at 60° C. to extract the PHB constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

Also, a detailed structure analysis by ¹H-NMR (FT-NMR: Bruker DPX400; ¹H resonance frequency: 400 MHz; measured nuclide: ¹H; solvent: CDCl₃; reference: capillary-sealed TMS/CDCl₃; measuring temperature: room temperature) confirmed, in addition to peaks derived from the PHA constituted of a 3-hydroxybutyric acid unit, peaks derived from polyethylene glycol at 3.5 to 3.8 ppm and about 4.2 ppm.

Further, the molecular weight of the PHB was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=22,000 and Mw=45,000.

### Example 8

A ferrite sheet of 30 mm×30 mm×3 mm (NP-S01, Nippon Paint Co., a dispersion of ferrite particles in resin) was immersed for 1 hour in 1% glutaraldehyde, then rinsed with purified water and immersed in the purified enzyme liquid (1) for 30 minutes at 30° C. to fix the enzyme. The unreacted scl-PHA synthetase was removed by rinsing with a PBS solution to obtain an enzyme-immobilized magnetic material.

The afore-described immobilized enzyme was immersed in 0.1 mol/L phosphate buffer (pH 7.0), containing 30 mmol/L of 3-hydroxybutyryl CoA (Sigma Aldrich Japan Co.) and 0.1% of bovine serum albumin (Sigma Co.) and the mixture was mildly shaken for 2 hours at 30° C. After the reaction, unreacted substance, etc., were removed by rinsing with 0.1 mol/L phosphate buffer (pH 7.0).

The ferrite sheet after the reaction was dyed with a 1% aqueous solution of Nile blue A and was observed under a fluorescence microscope (330 to 380 nm excitation filter, 420 nm long path absorption filter, Nikon Corp.). As a result, fluorescence from the surface of the ferrite sheet was observed to confirm that the construct was a laminar construct in which a base material of the ferrite sheet was covered by a PHB film.

Also, the laminar construct was dried under vacuum conditions and immersed in chloroform under agitation for 20 hours at 60° C. to extract the PHB constituting the coating layer. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified monomer unit. As a result, there was confirmed PHB constituted of a 3-hydroxybutyric acid unit.

### Example 9

Evaluation of Coating Property of Magnetic Capsule

### Construct

In order to confirm that the magnetic particles were completely protected and covered with the polymer, 0.1 g each of the obtained magnetic capsule constructs (1) to (7) were immersed for 2 hours in 100 ml of pure water heated to 70° C. and a metal content was measured in the liquid. As a result, the metal content in the water was 3 ppm or less with all the capsule constructs. Based on these facts, the capsule constructs were judged as follows: “metal ions did not elute.”

In the following, there will be shown examples relating to synthesizing enzymes for mcl-PHA and unusual-PHA (Reference Examples 5 to 7, Examples 11 to 28).

### Reference Example 5

Construction of Transformant Having mcl-PHA

### Synthetase Production Capacity

Strain YN2 was cultured in 100 ml of an LB medium (1% polypeptone (NIPPON SEIYAKU CO., LTD.), 0.5% yeast extract (Difco), 0.5% sodium chloride, pH 7.4) overnight at 30° C., and then, the chromosomal DNA was isolated by the method of Marmar et al. The obtained chromosomal DNA was completely digested by a restriction enzyme HindIII. A vector pUC18 was also cut by HindIII. After terminal dephosphorylation (Molecular Cloning, 1, 572, (1989); Cold Spring Harbor Laboratory Press (afore-described), complete HindIII digestion fragments of the chromosomal DNA were ligated to the HindIII cleavage site of the vector (a cloning site) using a DNA ligation kit Ver. II (TAKARA SHUZO CO., LTD.). With these plasmid vectors containing chromosomal DNA fragments, Escherichia coli HB 101 was transformed to construct a DNA library of strain YN2.

Next, to select DNA fragments covering the PHA synthetase gene of strain YN2, a probe for colony hybridization was prepared. Oligonucleotides of SEQ ID NO: 6 and SEQ ID NO: 7 were synthesized (Amersham Pharmacia Biotech), and PCR of chromosomal DNA of YN2 was carried out by using these oligonucleotides as primers. The PCR-amplified DNA fragments were used as a probe. Labeling of the probe was conducted by employing a commercially available labeling enzyme Alk Phos Direct (Amersham Pharmacia Biotech). Using the obtained labeled probe for colony hybridization, an Escherichia coli strain having the recombinant plasmid containing the mcl-PHA synthetase gene was selected from the chromosomal DNA library of YN2 by the colony hybridization method. The plasmid was recovered from the selected strain by the alkali process to give DNA fragment including the mcl-PHA synthetase gene.

This gene fragment was inserted into a vector pBBR122 having a wide-host-replication range (Mo Bi Tec) not belonging to any of IncP, IncQ, or IncW incompatibility group. When Pseudomonas cichorii YN2 ml (a PHA synthesis negative strain) was transformed with this recombinant plasmid by electroporation, the PHA synthesizing capacity of the YN2 ml strain was recovered to show complementarity.

Consequently, it was confirmed that the selected gene fragment contained mcl-PHA synthetase gene region translatable into mcl-PHA synthetase in Pseudomonas cichorii YN2 ml.

The base sequence of this DNA fragment was determined by the Sanger method. As a result, it was shown that there were base sequences represented by SEQ ID NO: 8 and SEQ ID NO: 9, each encoding a polypeptide. With respect to these mcl-PHA synthetase genes, PCR was carried out by using the chromosomal DNA as a template to produce the complete mcl-PHA synthetase gene. More specifically, an upstream primer (SEQ ID NO: 10) and a downstream primer (SEQ ID NO: 11) corresponding to the mcl-PHA synthetase gene of SEQ ID NO: 8 and an upstream primer (SEQ ID NO: 12) and a downstream primer (SEQ ID NO: 13) corresponding to the mcl-PHA synthetase gene of SEQ ID NO: 9 were synthesized, respectively (Amersham Pharmacia Biotech).

Using these primers, PCR was carried out for each of the base sequences shown by SEQ ID NO: 8 and SEQ ID NO: 9, and then, a full length of mcl-PHA synthetase gene was amplified (LA-PCR kit; TAKARA SHUZO CO., LTD.). Next, the obtained PCR amplified fragment and an expression vector pTrc99A were digested by the restriction enzyme HindIII and dephosphorylated (Molecular Cloning, vol. 1, p. 572, (1989); Cold Spring Harbor Laboratory Press), and then, the DNA fragment including a full length PHA synthetase gene excluding unnecessary base sequences at both terminuses was linked to a restriction site of the expression vector pTrc99A by using a DNA ligation kit Ver. II (TAKARA SHUZO CO., LTD.).

An E. coli strain (Escherichia coli HB101: TAKARA SHUZO) was transformed with each of the obtained recombinant plasmids by the calcium chloride method. The obtained recombinants were cultured and the recombinant plasmids were amplified. Then, the recombinant plasmids were respectively recovered. The recombinant plasmid having a DNA of SEQ ID NO: 8 was designated as pYN2-C1, and the recombinant plasmid having a DNA of SEQ ID NO: 9 was designated as pYN2-C2. An E. coli strain (Escherichia coli HB101fB fadB deletion strain) was transformed with pYN2-C1 and pYN2-C2, respectively, by the calcium chloride method to obtain recombinant E. coli strains having respective recombinant plasmids, i.e., a pYN2-C1 recombinant strain and a pYN2-C2 recombinant strain.

### Reference Example 6

### Production of mcl-PHA Synthetase 1

Construction of Transformant Having mcl-PHA

### Synthetase Production Capacity

For the pYN2-C1, an upstream primer (SEQ ID NO: 14) and a downstream primer (SEQ ID NO: 15) were designed and synthesized respectively (Amersham Pharmacia Biotech). PCR was carried out using these primers and template pYN2-C1 to synthesize a full length PHA synthetase gene having a BamHI restriction site upstream and a XhoI restriction site downstream (LA-PCR kit, TAKARA SHUZO CO., LTD.).

Similarly, for pYN2-C2, an upstream primer (SEQ ID NO: 16) and a downstream primer (SEQ ID NO: 17) were designed and synthesized, respectively (Amersham Pharmacia Biotech). PCR was carried out using these primers and the template pYN2-C2 to amplify the full length PHA synthetase gene having a BamHI restriction site upstream and a XhoI restriction site downstream (LA-PCR kit, TAKARA SHUZO CO., LTD.).

Respective purified PCR amplification products were digested by BamHI and XhoI and then inserted into the corresponding restriction sites of plasmid pGEX-6P-1 (Amersham Pharmacia Biotech). An E. coli strain JM109 was transformed with these vectors to obtain expressing strains. For confirmation, each plasmid DNA was prepared by Miniprep (Wizard Minipreps DNA Purification Systems, PROMEGA) in a large amount and digested by BamHI and XhoI, and the resulting DNA fragment was identified.

Preparation of Mcl-Pha Synthetase

Each obtained strain was pre-cultured in 10 ml of an LB-Amp medium overnight, and then, a 0.1 ml culture was transferred to 10 ml of the LB-Amp medium and cultured at 37° C. and 170 rpm for 3 hours under shaking. Then, IPTG was added to the culture (enc concentration 1 mmol/L), and then, the culture was continued for 4 to 12 hours at 37° C.

The E. coli cells induced with IPTG were collected (78,000 m/s² (=8,000 G), 2 minutes, 4° C.) and re-suspended in a 1/10 volume of phosphate buffer physiological saline (PBS; 8 g NaCl, 1.44 g Na₂HPO₄, 0.24 g KH₂PO₄, 0.2 g KCl, 1,000 ml purified water) at 4° C. The cells were disrupted by freeze, thawing and sonication, and subjected to centrifugation (78,000 m/s² (=8,000 G), 10 minutes, 4° C.) to remove solid impurities. Confirming that the aimed expressing protein was present in the supernatant by SDS-PAGE, the induced and expressed GST fusion protein was purified by using Glutathione Sepharose 4B (Amersham Pharmacia Biotech). The Glutathione Sepharose was previously treated to avoid nonspecific adsorption, that is, the Glutathione Sepharose was washed with an equivalent amount of PBS for three times (78,000 m/s² (=8,000 G), 1 minutes, 4° C.), and then, an equivalent amount of 4% bovine serum albumin PBS was added thereto and processed at 4° C. for one hour. After the process, the Sepharose was washed with an equivalent amount of PBS twice, and re-suspended in an 1/2 amount of PBS. The pre-treated 40 μl of Glutathione Sepharose was added to 1 ml of the above cell free extract and gently stirred at 4° C. to adsorb fusion proteins GST-YN2-C1 and GST-YN2-C2 onto Glutathione Sepharose, respectively. After centrifugation (78,000 M/s² (=8,000 G), 1 minutes, 4° C.) to collect the Glutathione Sepharose, it was washed with 400 μl of PBS for three times. Thereafter, 40 μl of 10 mmol/L of glutathione was added thereto and stirred for one hour at 4° C. to elute the adsorbed fusion protein. After centrifugation (78,000 m/s² (=8,000 G), 2 minutes, 4° C.), the supernatant was recovered and dialyzed against PBS to purify the GST fusion protein. A single band was confirmed by SDS-PAGE.

Then, 500 μg of each GST fusion protein was digested by PreScission protease (Amersham Pharmacia Biotech, 5U), and the protease and the GST were removed therefrom by passing through Glutathione Sepharose. The flow-through fraction was further loaded to the Sephadex G200 column equilibrated with PBS, and then, expression proteins YN2-C1 and YN2-C2 were obtained as final purified products. By SDS-PAGE, single bands (60.8 kDa and 61.5 kDa, respectively) were confirmed.

The above-described enzymes were concentrated with a bioliquid concentrating agent (Mizubutorikun AB-1100, Atto Corp.) to obtain 10 U/ml of purified enzyme solutions.

The enzyme activity was measured by the above-described method. The protein concentration of the sample was determined by using a micro BCA protein assay reagent kit (Pierce Chemical Co.). The results are shown in Table 1.

TABLE 1

| | Specific Activity |
|---|---|
| pYN2-C1 | 4.1 U/mg protein |
| pYN2-C2 | 3.6 U/mg protein |

### Reference Example 7

### Production of mcl-PHA Synthetase (2)

Each one of strains P91, H45, YN2, and P161 was inoculated in 200 ml of an M9 medium containing 0.5% of yeast extract (Difco) and 0.1% of octanoic acid, and incubated at 30° C. under shaking at 125 strokes/min. After 24 hours, the cells were harvested by centrifugation (98,000 m/s² (=10,000 G), 4° C., 10 minutes), and then, the cells were re-suspended in 200 ml of 0.1 mol/L Tris-HCl buffer (pH 8.0) and further subjected to centrifugation for washing. The cells were re-suspended in 2.0 ml of 0.1 mol/L Tris-HCl buffer (pH 8.0), disrupted by an ultrasonic homogenizer and then centrifuged (118,000 m/s² (=12,000 G), 4° C., 10 minutes) to collect the supernatant, thereby obtaining the crude enzyme.

Each purified enzyme activity was measured by the above-described method, and the results are shown in Table 2.

TABLE 2

| | Activity |
|---|---|
| P91 | 0.1 U/ml |
| H45 | 0.2 U/ml |
| YN2 | 0.4 U/ml |
| P161 | 0.2 U/ml |

### Example 11

### Preparation of Magnetic Capsule Construct (8)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), then 1 part by mass of 3-hydroxyoctanoyl CoA (prepared according to Eur. J. Biochem., 250, 432-439 (1997)) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (8).

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material (1) did not show fluorescence at all.

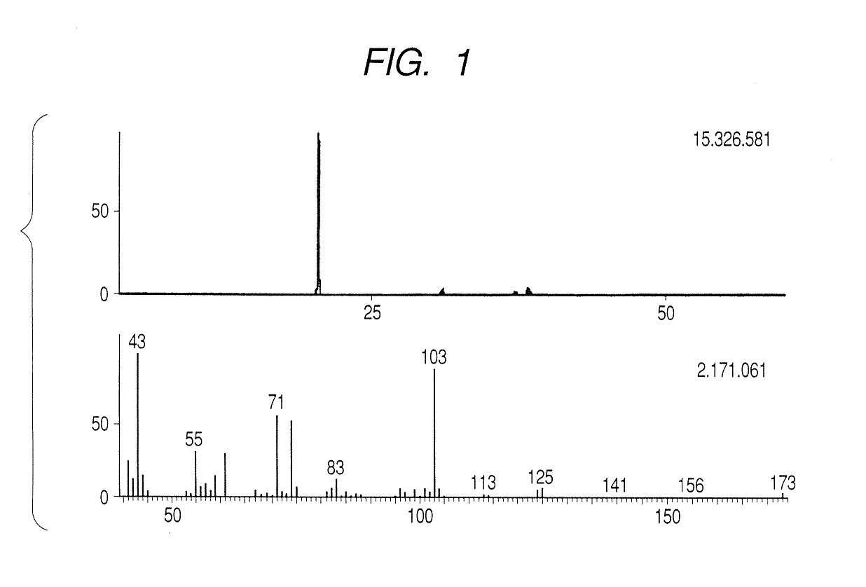

Also, a part of the synthesized PHA particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, as shown in FIG. 1, there was confirmed PHA constituted of a 3-hydroxyoctanoic acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=20,000 and Mw=39,000.

### Example 12

### Preparation of Magnetic Capsule Construct (9)

To 10 parts by mass of the mcl-PHA synthetase solution (10 U/ml) derived from pYN2-C2 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), an then, 1 part by mass of (R)-3-hydroxyoctanoyl CoA (prepared according to Eur. J. Biochem., 250, 432-439 (1997) (afore-described) and 0.1 parts by mass of bovine serum albumin (Sigma Co.) were added, and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (9).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, as in Example 11, there was confirmed PHA constituted of a 3-hydroxyoctanoic acid unit.

### Example 13

### Preparation of Magnetic Capsule Constructs (10) to (13)

To 99 parts by mass of the crude mcl-PHA synthetase derived from strain YN2, H45, P91 or P161, 1 part by mass of the magnetic material (1) was added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in PBS solution and centrifuged again (98,000 m/s² (=10,000 G), 4° C., 10 minutes) to obtain enzyme-immobilized magnetic material.

Each enzyme-immobilized magnetic material was suspended in 48 parts by mass of 0.1 mol/L phosphate buffer (pH 7.0), and then, 1 part by mass of (R)-3-hydroxyoctanoyl CoA (prepared according to Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, in any of the reaction liquids of the enzyme-immobilized magnetic materials, fluorescence from the surface of the magnetic material (1) was observed to confirm that the magnetic material (1) was coated with the PHA on the surface. These products were taken as magnetic capsule constructs (10) to (13).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, as in Example 11, there was confirmed PHA constituted of a 3-hydroxyoctanoic acid unit.

### Example 14

### Preparation of Magnetic Capsule Construct (14)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-phenylvaleryl CoA (prepared by hydrolyzing 3-hydroxyphenylvaleryl ester, obtained by a Reformatsky reaction, to obtain 3-hydroxy-5-phenylvaleric acid, and then following a method described in Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (14).

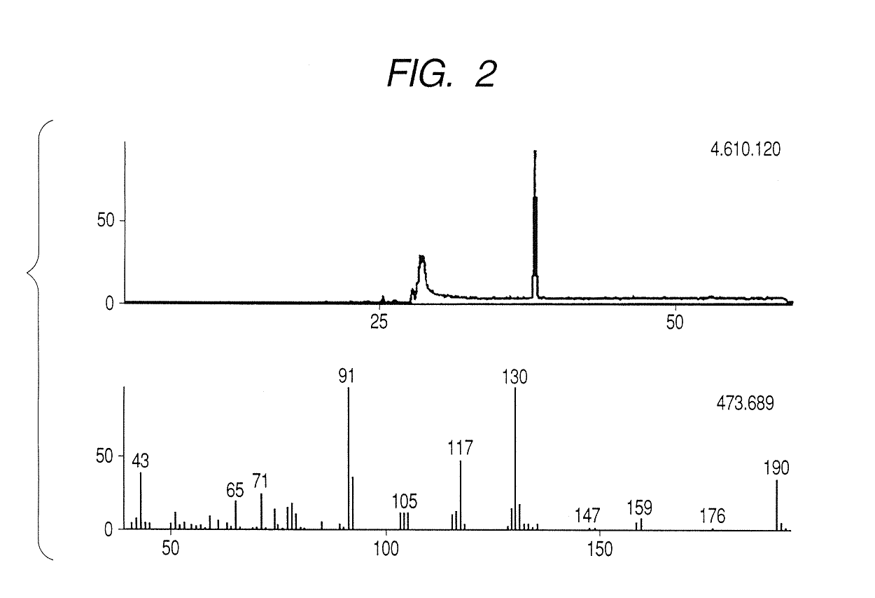

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, as shown in FIG. 2, there was confirmed PHA constituted of a 3-hydroxy-5-phenylvaleric acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=17,000 and Mw=35,000.

### Example 15

### Preparation of Magnetic Capsule Construct (15)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-(4-fluorophenyl)valeryl CoA (prepared by hydrolyzing 3-hydroxyphenylvaleryl ester, obtained by a Reformatsky reaction, to obtain 3-hydroxy-5-(4-fluorophenylvaleric) acid, and then following a method described in Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (15).

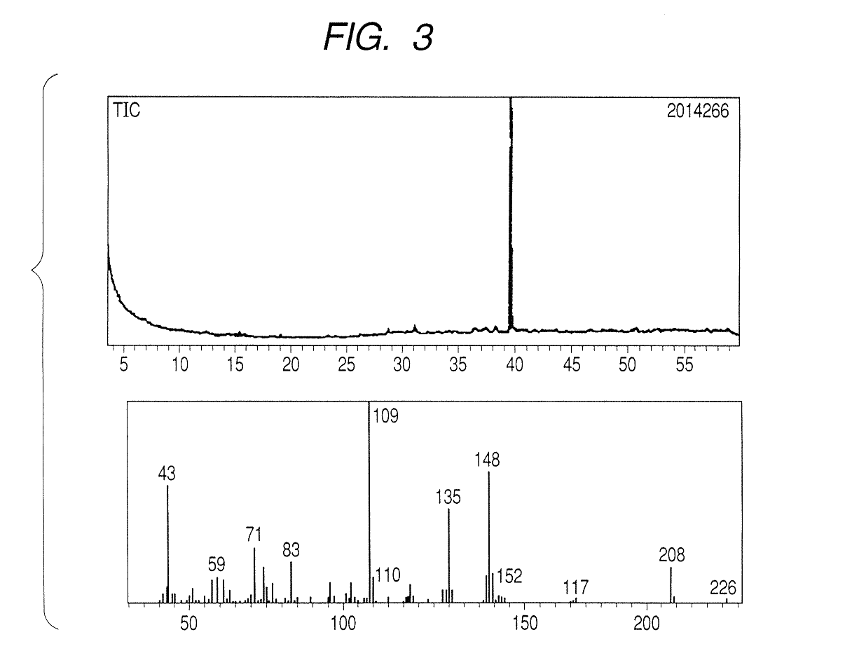

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, as shown in FIG. 3, there was confirmed PHA constituted of a 3-hydroxy-5-(4-fluorophenyl)valeric acid unit.

### Example 16

### Preparation of Magnetic Capsule Construct (16)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxyoctanoyl CoA (prepared according to Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (16).

Also, the capsule construct was recovered by a centrifugation (98,000 m/s² (=10,000 G), 4° C., 10 minutes), and, after drying, the mass of the polymer formed on the surface of the construct was measured by a time-of-flight secondary ion mass spectrometer (TOF-SIMS IV, CAMECA). The obtained mass spectrum confirmed that the surface of the capsule construct was principally comprised of a homopolymer of polyhydroxy octanoate. Also, the measurements of the similar TOF-SIMS mass spectrum under gradual scraping of the surface of the capsule construct by ion sputtering confirmed a polyhydroxyoctanoate homopolymer in all the cases. These results confirmed that the capsule construct of the present example was formed by coating the hydrophilic inorganic particles directly with the hydrophobic polyhydroxy octanoate homopolymer.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=25,000 and Mw=47,000.

### Example 17

### Preparation of Magnetic Capsule Construct (17)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxypimelyl CoA (prepared according to J. Bacteriol., 182, 2753-2760 (2000)) and 0.1 parts by mass of bovine serum albumin (Sigma Co.) were added and the mixture was mildly shaken for 10 minutes at 30° C. Then, to this reaction liquid under mild shaking at 30° C., a 0.1 mol/L phosphate buffer (pH 7.0), containing 1 part by mass of (R)-3-hydroxyoctanoyl CoA (prepared according to Eur. J. Biochem., 250, 432-439 (1997)) and 0.1 parts by mass of bovine serum albumin (Sigma Co.), was added at a rate of 1 part by mass per minute by a microtube pump (MP-3N, Tokyo Rikakikai Co.). After 1 hour and 30 minutes, the generated granulates were recovered by centrifugation (98000 m/s², 4° C., 10 minutes), and, after elimination of the supernatant, 25 parts by mass of 0.1 mol/L phosphate buffer (pH 7.0), containing 1 part by mass of (R)-3-hydroxyoctanoyl CoA (prepared according to Eur. J. Biochem., 250, 432-439 (1997)) and 0.1 part by mass of bovine serum albumin (Sigma Co.), were added to the granulates and the mixture was mildly shaken for 20 minutes at 30° C.

After the reaction, a 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (17).

Also, the capsule construct was recovered by a centrifugation (98,000 m/s² (=10,000 G), 4° C., 10 minutes), and, after drying, the mass of the polymer formed on the surface of the construct was measured by a time-of-flight secondary ion mass spectrometer (TOF-SIMS IV, CAMECA). The obtained mass spectrum confirmed that the surface of the capsule construct was comprised of a polyhydroxy octanoate homopolymer. Also, the measurements of the similar TOF-SIMS mass spectrum under gradual scraping of the surface of the capsule construct by ion sputtering revealed a copolymer of 3-hydroxyoctanoic acid and 3-hydroxypimelic acid (molar ratio 21:1), in which the proportion of 3-hydroxyoctanoic acid gradually decreased while that of 3-hydroxypimelic acid gradually increased toward the interior of the granulate, and which eventually changed to a polyhydroxy pimelate homopolymer. These results confirmed that the capsule construct of the present example was formed by coating the hydrophilic granular base material with polyhydroxy pimelate having a hydrophilic functional group, then with a copolymer of 3-hydroxy pimelic acid having a hydrophilic functional group and 3-hydroxy octanoic acid having a hydrophobic functional group with a gradually increasing proportion of 3-hydroxy octanoic acid toward the outer surface, and finally, coating the outermost layer with a polyhydroxy octanoate homopolymer.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=23,000 and Mw=43,000.

### Example 18

### Preparation of Magnetic Capsule Constructs (18) to (21)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 0.8 part by mass of (R,S)-3-hydroxy-5-phenoxyvaleryl CoA (prepared by hydrolyzing 3-hydroxy-5-phenoxyvaleryl ester, obtained by a Reformatsky reaction of 3-phenoxypropanal and ethyl bromoacetate, to obtain 3-hydroxy-5-phenoxyvaleric acid and then according to a process in Eur. J. Biochem., 250, 432-439 (1997)), 0.2 parts by mass of (R, S)-3-hydroxy-7,8-epoxyoctanoyl CoA (prepared by epoxylating an unsaturated part of 3-hydroxy-7-octenoic acid, prepared by a method in Int. J. Biol. Macromol., 12, 85-91 (1990) and then according to a process in Eur. J. Biochem., 250, 432-439 (1997)), and 0.1 parts by mass of bovine serum albumin (Sigma Co.) were added and the mixture was mildly shaken for 2 hours at 30° C. to obtain a magnetic capsule construct (18).

As a comparative reference, a magnetic capsule construct (19) was prepared in the same manner as explained above, except that (R, S)-3-hydroxy-7,8-epoxyoctanoyl CoA was replaced by 3-hydroxyoctanoyl CoA.

A 10 μl aliquot of the above sample was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, in each sample, fluorescence from the surface of the magnetic material (1) was observed to confirm that the magnetic material (1) was coated with the PHA on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material (1) did not show fluorescence at all.

Also, a part of the synthesized PHA particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from such a measurement are shown in Table 3.

TABLE 3

| | Magnetic | Magnetic |
|---|---|---|
| | Capsule | Capsule |
| Monomer Unit | Construct (18) | Construct (19) |
| 3-hydroxy-5-phenoxy | 75% | 71% |
| Valeric Acid | | |
| 3-hydroxy-7,8-epoxy | 25% | — |
| Octanoic Acid | | |
| 3-hydroxy Octanoic | — | 29% |
| Acid | | |

The operations of subjecting 50 parts by mass of the afore-described magnetic capsule construct (18) to centrifugation (98,000 M/s² (=10,000 G), 4° C., 10 minutes) to recover the capsule construct and suspending the same in 50 parts by mass of purified water and dissolving 0.5 part by mass of hexamethylene diamine in the suspension were repeated three times. After dissolution was confirmed, water was removed by lyophilization (thus providing a magnetic capsule construct (20)). Then, the magnetic capsule construct (20) was reacted for 12 hours at 70° C. (thus providing a magnetic capsule construct (21)).

Each of the magnetic capsule constructs (20) and (21) was suspended in chloroform and agitated for 20 hours at 60° C. to extract the PHA constituting the outer coating, which, after elimination of chloroform by drying under vacuum, was subjected to a differential scanning calorimeter (DSC; Perkin Elmer Inc., Pyris 1, temperature increase rate: 10° C./min). As a result, the magnetic capsule construct (20) showed a clear exothermic peak at about 90° C., indicating that a reaction between an epoxy group in the polymer and hexamethylene diamine took place and that the cross-linking between polymers was proceeding. On the other hand, the magnetic capsule construct (21) did not show a clear heat flow, indicating that the cross-linking reaction was almost completed.

Also, infrared absorption was measured on similar samples (FT-IR; Perkin Elmer, 1720×). As a result, peaks for amine (around 3340 cm⁻¹) and epoxy (around 822 cm⁻¹) observed in the magnetic capsule construct (20) disappeared in the magnetic capsule construct (21).

These results indicate that a cross-linked polymer can be obtained by reacting a PHA having an epoxy unit in the side chain and hexamethylene diamine.

On the other hand, a similar evaluation was conducted on the magnetic capsule construct (19) as a comparative reference, but a result clearly showing mutual cross-linking of polymers as explained above could not be obtained.

### Example 19

### Preparation of Magnetic Capsule Constructs (22) to (23)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 0.8 part by mass of (R,S)-3-hydroxy-5-phenoxyvaleryl CoA (prepared by hydrolyzing 3-hydroxy-5-phenoxyvaleryl ester, obtained by a Reformatsky reaction of 3-phenoxypropanal and ethyl bromoacetate, to obtain 3-hydroxy-5-phenoxyvaleric acid and then following a process in Eur. J. Biochem., 250, 432-439 (1997)), 0.2 parts by mass of (R, S)-3-hydroxy-7,8-epoxyoctanoyl CoA (prepared by epoxylating an unsaturated part of 3-hydroxy-7-octenoic acid, prepared by a process in Int. J. Biol. Macromol., 12, 85-91 (1990) and then following a process in Eur. J. Biochem., 250, 432-439 (1997)), and 0.1 parts by mass of bovine serum albumin (Sigma Co.) were added and the mixture was mildly shaken for 2 hours at 30° C. to obtain a magnetic capsule construct (22).

10 μl of the afore-described magnetic capsule construct (22) was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, in each sample, fluorescence from the surface of the magnetic material (1) was observed to confirm that the magnetic material (1) was coated with the PHA on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material (1) did not show fluorescence at all.

Also, a part of the synthesized PHA particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from such a measurement were 78% for 3-hydroxy-5-phenoxyvaleric acid and 22% for 3-hydroxy-7,8-epoxyoctanoic acid.

The operations of subjecting 50 parts by mass of the afore-described magnetic capsule construct (22) to centrifugation (98,000 M/s² (=10,000 G), 4° C., 10 minutes) to recover the capsule construct and suspending the same in 50 parts by mass of purified water were repeated three times, and water was removed by lyophilization. Then, 10 parts by mass of terminal-amino modified polysiloxane (modified silicone oil TSF4700, GE Toshiba Silicone Co.) were added and reacted for 2 hours at 70° C. The construct was rinsed by repeating operations of suspending methanol and centrifuging (98,000 M/s² (=10,000 G), 4° C., 20 minutes) and dried to obtain a magnetic capsule construct (23) having a polysiloxane graft chain.

### Example 20

### Preparation of Magnetic Capsule Construct (24)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 M/s² (=10,000 G), 4° C., 10 minutes), and the precipitate was suspended in a PBS solution and centrifuged again (98,000 M/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 0.8 part by mass of (R,S)-3-hydroxy-5-phenoxyvaleryl CoA (prepared by hydrolyzing 3-hydroxy-5-phenoxyvaleryl ester, obtained by a Reformatsky reaction of 3-phenoxypropanal and ethyl bromoacetate, to obtain 3-hydroxy-5-phenoxyvaleric acid and then following a process in Eur. J. Biochem., 250, 432-439 (1997)), 0.2 parts by mass of (R)-3-hydroxypimelyl CoA (prepared according to J. Bacteriol., 182, 2753-2760 (2000)), and 0.1 parts by mass of bovine serum albumin (Sigma Co.) were added and the mixture was mildly shaken for 2 hours at 30° C. to obtain a magnetic capsule construct (24).

A 10 μl aliquot of the afore-described magnetic capsule construct (24) were put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface.

Also, a part of the magnetic capsule construct (24) was collected by centrifugation (98,000 M/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from such a measurement were 83% for 3-hydroxy-5-phenoxyvaleric acid and 17% for 3-hydroxypimelic acid.

### Example 21

### Preparation of Magnetic Capsule Construct (25)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 M/s² (=10,000 G), 4° C., 10 minutes), and the precipitate was suspended in a PBS solution and centrifuged again (98,000 M/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 0.8 part by mass of (R,S)-3-hydroxy-5-phenoxyvaleryl CoA (prepared by hydrolyzing 3-hydroxy-5-phenoxyvaleryl ester, obtained by a Reformatsky reaction of 3-phenoxypropanal and ethyl bromoacetate, to obtain 3-hydroxy-5-phenoxyvaleric acid and then following a process in Eur. J. Biochem., 250, 432-439 (1997)), 0.2 parts by mass of (R)-3-hydroxy-8-bromooctanoyl CoA (prepared according to Eur. J. Biochem., 250, 432-439 (1997)), and 0.1 parts by mass of bovine serum albumin (Sigma Co.) were added and the mixture was mildly shaken for 2 hours at 30° C. to obtain a magnetic capsule construct (25).

A 10 μl aliquot of the afore-described magnetic capsule construct (25) was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface.

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from such a measurement were 89% for 3-hydroxy-5-phenoxyvaleric acid and 11% for 3-hydroxy-8-bromooctanoic acid.

### Example 22

### Preparation of Magnetic Capsule Construct (26)

To 10 parts by mass of the mcl-PHA synthetase solution (10 U/ml) derived from pYN2-C1 recombinant strain, 1 part by mass of nickel powder of a primary particle size of 0.02 μm (Ni(200)UFMP, Shinku Yakin Co.) (magnetic material (2)) as magnetic metal synthesized by a gaseous method, and 39 parts by mass of PBS were added and mildly shaken for 30 minutes at 30° C. to cause the mcl-PHA synthetase to be adsorbed on the surface of the magnetic material (2). The mixture was centrifuged (98,000 m/s² (=10,000 G) 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-phenylvaleryl CoA (prepared by hydrolyzing 3-hydroxyphenylvaleryl ester to obtain 3-hydroxy-5-phenylvaleric acid and then following a process in J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (2) was observed to confirm that the magnetic material (2) was coated with the PHA on the surface. This product was taken as a magnetic capsule construct (26).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, there was confirmed PHA constituted of a 3-hydroxy-5-phenylvaleric acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=18,000 and Mw=36,000.

### Example 23

### Preparation of Magnetic Capsule Construct (27)

To 10 parts by mass of the mcl-PHA synthetase solution (10 U/ml) derived from pYN2-C1 recombinant strain, 1 part by mass of γ-Fe₂O₃ fine powder of a primary particle size of 0.02 μm (NanoTek, CI Chemical Co.) synthesized via a gaseous method (magnetic material (3)), and 39 parts by mass of PBS were added and mildly shaken for 30 minutes at 30° C. to cause the mcl-PHA synthetase to be adsorbed on the surface of the magnetic material (3). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-phenylvaleryl CoA (prepared by hydrolyzing 3-hydroxyphenylvaleryl ester to obtain 3-hydroxy-5-phenylvaleric acid and then following a process in Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (3) was observed to confirm that the magnetic material (3) was coated with the PHA on the surface. This product was taken as a magnetic capsule construct (27).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, there was confirmed PHA constituted of a 3-hydroxy-5-phenylvaleric acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=17,000 and Mw=35,000.

### Example 24

### Preparation of Magnetic Capsule Construct (28)

To 10 parts by mass of the mcl-PHA synthetase solution (10 U/ml) derived from pYN2-C1 recombinant strain, 1 part by mass of magnetite fine powder of a primary particle size of 0.3 μm (Magnetite EPT500, Toda Kogyo Co.) synthesized by a wet process (magnetic material (4)), and 39 parts by mass of PBS were added and mildly shaken for 30 minutes at 30° C. to cause the mcl-PHA synthetase to be adsorbed on the surface of the magnetic material (4). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-phenylvaleryl CoA (prepared by hydrolyzing 3-hydroxyphenylvaleryl ester to obtain 3-hydroxy-5-phenylvaleric acid and then following a process in Eur. J. Biochem., 250, 432-439 (1997)) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (4) was observed to confirm that the magnetic material (4) was coated with the PHA on the surface. This product was taken as a magnetic capsule construct (28).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, there was confirmed PHA constituted of a 3-hydroxy-5-phenylvaleric acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=15,000 and Mw=34,000.

### Example 25

### Preparation of Magnetic Capsule Construct (29)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxyoctanoyl CoA (prepared according to Eur. J. Biochem., 250, 432-439 (1997)), 1 part by mass of polyethylene glycol 200 (PEG200, Kishida Kagaku Co., average molecular weight 190 to 210) and 0.1 parts by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material (1) did not show fluorescence at all.

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, there was confirmed PHA constituted of a 3-hydroxyoctanoic acid unit.

Also, a detailed structure analysis by ¹H-NMR (FT-NMR: Bruker DPX400; ¹H resonance frequency: 400 MHz; measured nuclide: ¹H; solvent: CDCl₃; reference: capillary-sealed TMS/CDCl₃; measuring temperature: room temperature) confirmed, in addition to peaks derived from the PHA constituted of a 3-hydroxyoctanoic acid unit, peaks derived from polyethylene glycol at 3.5 to 3.8 ppm and about 4.2 ppm.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the result of Mn=7,000 and Mw=13,000, and a molecular weight reducing effect was obtained.

### Example 26

### Preparation of Laminar Construct

A ferrite sheet of 30 mm×30 mm×3 mm (NP-S01, Nippon Paint Co., a dispersion of ferrite particles in resin) was immersed for 1 hour in 1% glutaraldehyde, then rinsed with pure water and immersed in a solution (10 U/ml) of mcl-PHA synthetase derived from pYN2-C1 strain for 30 minutes at 30° C. to fix the enzyme. The unreacted mcl-PHA synthetase was removed by rinsing with a PBS solution to obtain an enzyme-immobilized magnetic material.

The afore-described immobilized enzyme was immersed in 0.1 mol/L phosphate buffer (pH 7.0), containing 30 mmol/L of 3-hydroxyoctanoyl CoA (prepared according to Eur. J. Biochem., 250, 432-439 (1997)), and 0.1% of bovine serum albumin (Sigma Co.) and the mixture was mildly shaken for 2 hours at 30° C. After the reaction, unreacted substance, etc., were removed by rinsing with 0.1 mol/L phosphate buffer (pH 7.0).

The ferrite sheet after the reaction was dyed with a 1% aqueous solution of Nile blue A and was observed under a fluorescence microscope (330 to 380 nm excitation filter, 420 nm long path absorption filter, Nikon Corp.). As a result, fluorescence from the surface of the ferrite sheet was observed to confirm that the construct was a laminar construct in which a base material of the ferrite sheet was covered by a PHA film.

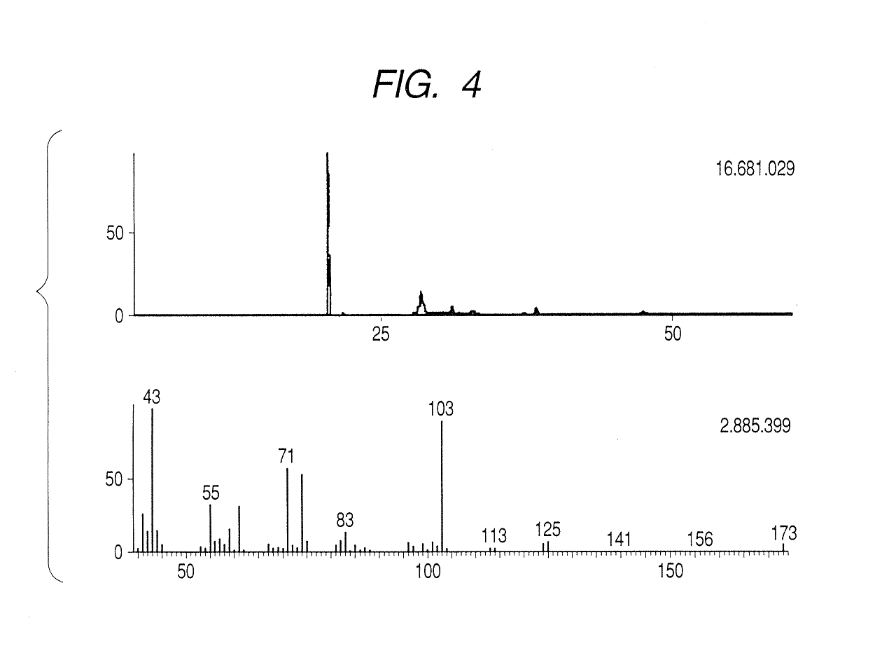

Also, the laminar construct was dried in a vacuum and immersed in chloroform under agitation for 20 hours at 60° C. to extract the PHA constituting the coating layer. The extract was filtered through a membrane filter of 0.45 μm pore size and concentrated under a reduced pressure by using a rotary evaporator. Then, the extract was subjected to methanolysis by a conventional method and analyzed by gas chromatography-mass spectroscopy (GC-MS, Shimadzu QP-5050, an EI method) to identify the methyl-esterified PHA monomer unit. As a result, there was confirmed PHA constituted of a 3-hydroxyoctanoic acid unit, as shown in FIG. 4.

### Example 27

### Evaluation of Coating Property of Magnetic Capsule Construct

In order to confirm that the magnetic particles were completely protected and covered with the polymer, 0.1 g each of the obtained magnetic capsule constructs (8) to (29) was immersed for 2 hours in 100 ml of purified water heated to 70° C. and a metal content in the water was measured. As a result, the metal content was 3 ppm or less for all the capsule constructs. Based on these facts, the capsule constructs were judged as follows: “metal ions did not elute.”

### Example 29

### Preparation of Magnetic Capsule Construct (30)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), then 1 part by mass of (R)-3-hydroxy-5-(4-vinylphenyl)valeryl CoA (prepared according to Eur. J. Biochem., 250, 432-439 (1997)), and 0.1 parts by mass of bovine serum albumin (Sigma Co.) were added and the mixture was mildly shaken for 2 hours at 30° C.

After the reaction, a 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface. This product was taken as a magnetic capsule construct (30).

Also, a part of the particles was collected by centrifugation (98,000 M/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). As a result, it was confirmed that the PHA was constituted of a (R)-3-hydroxy-5-(4-vinylphenyl)valeric acid unit.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=21,000 and Mw=39,000.

### Example 30

### Preparation of Magnetic Capsule Construct (31)

To 10 parts by mass of the mcl-PHA synthetase solution (10 U/ml) derived from pYN2-C1 recombinant strain, 1 part by mass of nickel powder of a primary particle size of 0.02 μm (Ni(200)UFMP, Shinku Yakin Co.) (magnetic material (2)) as a magnetic metal synthesized by a gaseous method, and 39 parts by mass of PBS were added and mildly shaken for 30 minutes at 30° C. to cause the mcl-PHA synthetase to be adsorbed on the surface of the magnetic material (2). The mixture was centrifuged (98,000 M/s² (=10,000 G), 4° C., 10 minutes), and the precipitate was suspended in a PBS solution and centrifuged again (98,000 M/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-(4-methylphenoxy)valeryl CoA (prepared according to Eur. J. Biochem., 250, 432-439 (1997)) and 0.1 part by mass of bovine serum albumin (Sigma Co.) were added and the mixture was mildly shaken for 10 minutes at 30° C. Then, to this reaction liquid under mild shaking at 30° C., a 0.1 mol/L phosphate buffer (pH 7.0), containing 1 part by mass of (R)-3-hydroxy-5-(4-methylphenyl)valeryl CoA (prepared according to Eur. J. Biochem., 250, 432-439 (1997)) and 0.1 parts by mass of bovine serum albumin (Sigma Co.), was added at a rate of 1 part by mass per minute by a microtube pump (MP-3N, Tokyo Rikakikai Co.). After 1 hour and 30 minutes, the generated granulates were recovered by centrifugation (98,000 m/s², 4° C., 10 minutes), and, after elimination of the supernatant, 25 parts by mass of 0.1 mol/L phosphate buffer (pH 7.0), containing 1 part by mass of (R)-3-hydroxy-5-(4-methylphenyl)valeryl CoA (prepared according to Eur. J. Biochem., 250, 432-439 (1997)) and 0.1 part by mass of bovine serum albumin (Sigma Co.), were added to the granulates and the mixture was mildly shaken for 20 minutes at 30° C.

After the reaction, a 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (2) was observed to confirm that the magnetic material (2) was coated with the PHA on the surface. This product was taken as a magnetic capsule construct (31).

Also, the capsule construct was recovered by a centrifugation (98,000 m/s² (=10,000 G), 4° C., 10 minutes), and, after drying, the mass of the polymer formed on the surface of the construct was measured by a time-of-flight secondary ion mass spectrometer (TOF-SIMS IV, CAMECA). The obtained mass spectrum confirmed that the surface of the capsule construct was principally comprised of a homopolymer of polyhydroxy (4-methylphenyl)valeric acid. Also, the measurements of the similar TOF-SIMS mass spectrum under gradual scraping of the surface of the capsule construct by ion sputtering showed a copolymer of 3-hydroxy-5-(4-methylphenyl)valeric acid and 3-hydroxy-5-(4-methylphenoxy)valeric acid, in which the proportion of 3-hydroxy-5-(4-methylphenyl)valeric acid gradually decreased while that of 3-hydroxy-5-(4-methylphenoxy)valeric acid gradually increased toward the interior of the granulate, and which eventually changed to a polyhydroxy (4-methylphenoxy)valeric acid homopolymer. These results confirmed that the capsule construct of the present example was formed by coating the surface of base material with a high polarity polyhydroxy(4-methylphenoxy)valeric acid, then with a copolymer of 3-hydroxy-5-(4-methylphenoxy)valeric acid and 3-hydroxy-5-(4-methylphenyl)valeric acid with a gradually increasing proportion of 3-hydroxy-5-(4-methylphenyl)valeric acid toward the outer surface, and finally, coating the outermost layer with a low polarity polyhydroxy(4-methylphenyl)valeric homopolymer acid.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the following results: Mn=17,000 and Mw=36,000.

### Example 31

### Preparation of Magnetic Capsule Construct (32)

To 10 parts by mass of the mcl-PHA synthetase solution (10 U/ml) derived from pYN2-C1 recombinant strain, 1 part by mass of γ-Fe₂O₃ fine powder of a primary particle size of 0.02 μm (NanoTek, CI Chemical Co.) synthesized in gaseous method (magnetic material (3)), and 39 parts by mass of PBS were added and mildly shaken for 30 minutes at 30° C. to cause the mcl-PHA synthetase to be adsorbed on the surface of the magnetic material (3). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-phenylsulfanylvaleryl CoA (prepared according to Eur. J. Biochem., 250, 432-439 (1997))) and 0.1 part by mass of bovine serum albumin (Sigma Co.) were added and the mixture was mildly shaken for 2 hours at 30° C.

After the reaction, a 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (3) was observed to confirm that the magnetic material (3) was coated with the PHA on the surface. This product was taken as a magnetic capsule construct (32).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). As a result, it was confirmed that PHA was constituted of a (R)-3-hydroxy-5-phenylsulfanylvaleric acid unit.

### Example 32

### Preparation of Magnetic Capsule Constructs (33) and (34)

To 10 parts by mass of the mcl-PHA synthetase solution (10 U/ml) derived from pYN2-C1 recombinant strain, 1 part by mass of magnetite fine powder of a primary particle size of 0.3 μm (Magnetite EPT500, Toda Kogyo Co.) synthesized by a wet process (magnetic material (4)), and 39 parts by mass of PBS were added and mildly shaken for 30 minutes at 30° C. to cause the mcl-PHA synthetase to be adsorbed on the surface of the magnetic material (4). The mixture was centrifuged (98,000 m/s² (=10,000 G), 4° C., 10 minutes), and the precipitate was suspended in a PBS solution and centrifuged again (98,000 M/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 0.8 parts by mass of (R)-3-hydroxy-5-phenylvaleryl CoA (prepared according to Eur. J. Biochem., 250, 432-439 (1997)), 0.2 parts by mass of (R)-3-hydroxy-5-(4-vinylphenyl)valeryl CoA (prepared according to Eur. J. Biochem., 250, 432-439 (1997)), and 0.1 parts by mass of bovine serum albumin (Sigma Co.) were added and the mixture was mildly shaken for 2 hours at 30° C. to obtain a sample 1.

As a comparative reference, a sample 2 was obtained in the same process as explained above, except that (R)-3-hydroxy-5-(4-vinylphenyl)valeryl CoA was replaced by (R)-3-hydroxy-5-(4-methylphenyl)valeryl CoA (prepared according to Eur. J. Biochem., 250, 432-439 (1997)).

A 10 μl aliquot of each sample was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (4) was observed to confirm that the magnetic material (4) was coated with the PHA on the surface. These products were taken as a magnetic capsule constructs (33) and (34).

As a control, 1 part by mass of the magnetic material (4) was added to 49 parts by mass of 0.1 mol/L phosphate buffer (pH 7.0), then, after mild shaking for 2.5 hours at 30° C., similarly dyed with Nile blue A and observed under a fluorescence microscope. As a result, the surface of the magnetic material (4) did not show fluorescence at all.

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from the measured results are shown in Table 4.

TABLE 4
Composition of PHA of external coating of capsule
construct (¹H-NMR, unit %)

| | Magnetic | Magnetic |
|---|---|---|
| | Capsule | Capsule |
| Monomer Unit | Construct 33 | Construct 34 |
| 3-hydroxy-5-valeric Acid | 83% | 85% |
| 3-hydroxy-5-(4- | 17% | — |
| vinylphenyl) Valeric Acid | | |
| 3-hydroxy-5-(4- | — | 15% |
| methylphenyl) Valeric | | |
| Acid | | |

### Example 33

### Preparation of Capsule Construct 35

An epoxylating reaction was conducting on the magnetic capsule construct prepared in Example 32. 1 part by mass of the magnetic capsule construct 4 was charged in a four-necked flask and was agitated with 6 parts by mass of distilled water. The interior of the flask was heated to 40° C., and 1 part by mass of a 30% hexane solution of peracetic acid was drop-wise added continuously and was reacted for 5 hours under agitation at 40° C. The reaction proceeded without mutual coagulation of the particles of the magnetic capsule construct. After the reaction, the reaction liquid was cooled to the room temperature and filtered to recover the magnetic capsule construct. The recovered magnetic capsule construct was re-suspended in distilled water and centrifuged (29,400 M/s² (=3,000 G), 4° C., 30 minutes). After the separation, the magnetic capsule construct was rinsed by suspending in distilled water again and centrifuging again. This rinsing operation was repeated three times. Thereafter, drying under vacuum conditions was conducted to obtain a desired magnetic capsule construct (35).

Also, a part of the particles was collected by centrifugation (98,000 M/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from the measured results were 85% for 3-hydroxy-5-phenylvaleric acid, 11% for 3-hydroxy-5-(4-vinylphenyl)valeric acid and 4% for 3-hydroxy-5-(4-epoxyphenyl)valeric acid.

Since this reaction system was an inhomogeneous reaction, it is estimated that the surface of the capsule construct was epoxylated while the interior of the coating layer remained unreacted.

### Example 34

Cross-linking Reaction of Capsule Construct

The operations of subjecting 50 parts by mass of the magnetic capsule construct (35) prepared in the Example 33 to centrifugation (98,000 M/s² (=10,000 G), 4° C., 10 minutes) to recover the capsule construct and suspending the same in 50 parts by mass of purified water were repeated three times, and 0.5 parts by mass of hexamethylene diamine were dissolved in the suspension. After dissolution was confirmed, water was removed by lyophilization (thus providing a magnetic capsule construct (36)). Then, the magnetic capsule construct (36) was reacted for 12 hours at 70° C. (thus providing a magnetic capsule construct (37)).

Each of the magnetic capsule constructs (36) and (37) was suspended in chloroform and agitated for 20 hours at 60° C. to extract the PHA constituting the outer coating, which, after elimination of chloroform by drying under vacuum conditions, was subjected to a differential scanning calorimeter (DSC; Perkin Elmer Inc., Pyris 1, temperature increase rate: 10° C./min). As a result, the magnetic capsule construct (36) showed a clear exothermic peak at about 90° C., indicating that a reaction between an epoxy group in the polymer and hexamethylene diamine took place and the cross-linking between polymers was proceeding. On the other hand, the magnetic capsule construct (37) did not show a clear heat flow, indicating that the cross-linking reaction was almost completed.

Also, infrared absorption was measured on similar samples (FT-IR; Perkin Elmer, 1720×). As a result, peaks for amine (around 3.340 cm⁻¹) and epoxy (around 822 cm⁻¹) observed in the magnetic capsule construct (36) disappeared in the magnetic capsule construct (37).

These results indicate that a cross-linked polymer can be obtained by reacting the PHA having an epoxy unit in the side chain and hexamethylene diamine.

On the other hand, a similar evaluation of the magnetic capsule construct (34) was conducted as a comparative reference, but a result clearly showing the mutual cross-linking of polymers as explained above was not obtained.

### Example 35

### Grafting of Capsule Construct

The operations of subjecting 50 parts by mass of the afore-described magnetic capsule construct (35) prepared in the Example 33 to centrifugation (98,000 m/s² (=10,000 G), 4° C., 10 minutes) to recover the capsule construct and suspending the same in 50 parts by mass of purified water were repeated three times, and water was removed by lyophilization. Then, 10 parts by mass of terminal-amino modified polysiloxane (modified silicone oil TSF4700, GE Toshiba Silicone Co.) was added and reacted for 2 hours at 70° C. The construct was rinsed by repeating operations of suspending methanol and centrifuging (98,000 m/s² (=10,000 G), 4° C., 20 minutes) and dried to obtain a magnetic capsule construct (38) having a polysiloxane graft chain.

### Example 36

### Preparation of Capsule Construct

An oxidation cleaving reaction of the vinyl group was conducted on the magnetic capsule construct (33) prepared in the Example 32. 10 parts by mass of the magnetic capsule construct was transferred to a three-necked flask, and 300 parts by mass of distilled water containing 50 ppm of hydrogen peroxide was added. Agitation was conducted for 3 hours at room temperature, under blowing of ozone at a rate of 1 part by mass per hour. The reaction proceeded without coagulation of the particles of the magnetic capsule construct. After the reaction, the reaction liquid was filtered to recover the magnetic capsule construct. The magnetic capsule construct was suspended again in distilled water and was centrifuged (29,400 m/s² (=3,000 G), 4° C., 30 minutes) to wash off the remaining hydrogen peroxide-containing water. This rinsing operation was repeated twice. Thereafter, drying under vacuum conditions was conducted to obtain a magnetic capsule construct (39).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from the measured results were 84% for 3-hydroxy-5-phenylvaleric acid, 11% for 3-hydroxy-5-(4-vinylphenyl)valeric acid and 5% for 3-hydroxy-5-(4-carboxyphenyl)valeric acid.

Since this reaction system was an inhomogeneous reaction, it was estimated that the surface of the capsule construct was subjected to oxidation cleaving while the interior of the coating layer remained unreacted.

### Example 37

### Preparation of Capsule Construct (40)

1 part by mass of the magnetic capsule construct (32) prepared in the Example 31 was added to 150 parts by mass of a commercially available aqueous solution of hydrogen peroxide (Mitsubishi Gas Chemical Co., containing 31% of hydrogen peroxide, according to standard JIS K-8230) and 30 parts by mass of deionized water, and the mixture was transferred to an eggplant-shaped flask and was reacted on an oil bath for 1 hour at 100° C. After the reaction, it was cooled to the room temperature, and the magnetic capsule construct was separated by centrifugation (29,400 m/s² (=3,000 G), 4° C., 10 minutes). The magnetic capsule construct was suspended again in distilled water and was centrifuged again to wash off the remaining hydrogen peroxide-containing water. This rinsing operation was repeated twice. Thereafter, drying under vacuum was conducted to obtain a magnetic capsule construct (40).

Also, a part of the particles was collected by centrifugation (98,000 M/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from the measured results were 64% for 3-hydroxy-5-phenylsulfanylvaleric acid, 16% for 3-hydroxy-5-phenylsufinylvaleric acid and 20% for 3-hydroxy-5-phenylsulfonylvaleric acid.

Since this reaction system was an inhomogeneous reaction, it was estimated that the surface of the capsule construct was subjected to oxidation while the interior of the coating layer remained unreacted.

### Example 38

### Preparation of Capsule Construct (41)

1 part by mass of the magnetic capsule construct (32) prepared in the Example 31 was added to 90 parts by mass of a commercially available aqueous solution of hydrogen peroxide (Mitsubishi Gas Chemical Co., containing 31% of hydrogen peroxide, according to standard JIS K-8230) and 30 parts by mass of deionized water, and the mixture was transferred to an eggplant-shaped flask and was reacted on an oil bath for 1 hour at 100° C. After the reaction, it was cooled to the room temperature, and the magnetic capsule construct was separated by centrifugation (29,400 m/s² (=3,000 G), 4° C., 10 minutes). The magnetic capsule construct was suspended again in distilled water and was centrifuged again to wash off the remaining hydrogen peroxide-containing water. This rinsing operation was repeated twice. Thereafter, drying under vacuum was conducted to obtain a magnetic capsule construct (41).

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). Percentages of the side chain units calculated from the measured results were 61% for 3-hydroxy-5-phenylsulfanylvaleric acid, 31% for 3-hydroxy-5-phenylsufinylvaleric acid and 8% for 3-hydroxy-5-phenylsulfonylvaleric acid.

Since this reaction system was an inhomogeneous reaction, it was estimated that the surface of the capsule construct was subjected to oxidation while the interior of the coating layer remained unreacted.

### Example 39

### Preparation of Magnetic Capsule Construct (42)

To 10 parts by mass of the mcl-PHA synthetase solution (10 U/ml) derived from pYN2-C1 recombinant strain, 1 part by mass of the magnetic material (1) and 39 parts by mass of PBS were added and mildly shaken for 30 minutes at 30° C. to cause the mcl-PHA synthetase to be adsorbed on the surface of the magnetic material (1). The mixture was centrifuged (98,000 m/s² (=10,000 G), 4° C., 10 minutes), and the precipitate was suspended in a PBS solution and centrifuged again (98,000 m/s² (=10,000 G), 4° C., 10 minutes) to obtain an enzyme-immobilized magnetic material.

The afore-described immobilized enzyme was suspended in 48 parts by mass of 0.1 mol/L phosphate buffer (pH 7.0), and then, 1 part by mass of (R)-3-hydroxy-5-(4-vinylphenyl)valeryl CoA (prepared according to Eur. J. Biochem., 250, 432-439 (1997)), 1 part by mass of polyethylene glycol 200 (PEG200, Kishida Kagaku Co., average molecular weight 190 to 210) and 0.1 part by mass of bovine serum albumin (Sigma Co.) were added and the mixture was mildly shaken for 2 hours at 30° C.

A 10 μl aliquot of the above reaction solution was put on a slide glass, to which 10 μl of a 1% solution of Nile blue A in water was added. These solutions were mixed on the slide glass, covered with a cover glass, and observed under a fluorescence microscope (330 to 380 nm excitation filter, 420 nm long path absorption filter, Nikon Corp.). As a result, fluorescence from the surface of the magnetic material (1) was observed to confirm that the magnetic material (1) was coated with the PHA on the surface.

As a control, 1 part by mass of the magnetic material (1) was added to 49 parts by mass of 0.1 mol/L phosphate buffer (pH 7.0), and then, after mild shaking for 2.5 hours at 30° C., similarly dyed with Nile blue A and observed under a fluorescence microscope. As a result, the surface of the magnetic material (1) did not show fluorescence at all.

Also, a part of the particles was collected by centrifugation (98,000 m/s² (=10,000 G), 4° C., 10 minutes), dried in a vacuum, suspended in chloroform and agitated for 20 hours at 60° C. to extract the PHA constituting the external coating. The extract was subjected to an analysis by ¹H-NMR (equipment: FT-NMR: Bruker DPX400; measured nuclide: ¹H; solvent: deuterated chloroform (containing TMS)). As a result, PHA was confirmed to be constituted of a (R)-3-hydroxy-5-(4-vinylphenyl)valeric acid unit. Also, there were confirmed, in addition to peaks derived from the (R)-3-hydroxy-5-(4-vinylphenyl)valeric acid unit, peaks derived from PEG at 3.5 to 3.8 ppm and at about 4.2 ppm.

Further, the molecular weight of the PHA was evaluated by gel permeation chromatography (GPC: Toso HLC-8020, column: Polymer Laboratory PLgel MIXED-C (5 μm), solvent: chloroform, column temperature: 40° C., converted as polystyrene) to obtain the results of Mn=7,700 and Mw=14,000, and a molecular weight reducing effect was obtained.

### Example 40

### Preparation of Laminar Construct

A ferrite sheet of 30 mm×30 mm×3 mm (NP-S01, Nippon Paint Co., a dispersion of ferrite particles in resin) was immersed for 1 hour in 1% glutaraldehyde and then rinsed with purified water and immersed in a solution (10 U/ml) of a PHA synthetase derived from pYN2-C1 strain for 30 minutes at 30° C. to fix the enzyme. The unreacted PHA synthetase was removed by rinsing with a PBS solution to obtain an enzyme-immobilized magnetic material.

The afore-described immobilized enzyme was immersed in 0.1 mol/L phosphate buffer (pH 7.0), containing 30 mmol/L of (R)-3-hydroxy-5-(4-methylthio(phenoxy))valeryl CoA (prepared according to Eur. J. Biochem., 250, 432-439 (1997)) and 0.1% of bovine serum albumin (Sigma Co.) and the mixture was mildly shaken for 2 hours at 30° C. After the reaction, unreacted substance, etc., were removed by rinsing with 0.1 mol/L phosphate buffer (pH 7.0).

The sheet after the reaction was dyed with a 1% aqueous solution of Nile blue A and was observed under a fluorescence microscope (330 to 380 nm excitation filter, 420 nm long path absorption filter, Nikon Corp.). As a result, fluorescence from the surface of the sheet was observed to confirm that the construct was a laminar construct in which a base material of the ferrite sheet was covered by a PHA film.

Also, the laminar construct was dried in a vacuum, and immersed in chloroform under agitation for 20 hours at 60° C. to extract the PHA constituting the coating layer. The extract was subjected to an analysis by ¹H-NMR (equipment: FT-NMR: Bruker DPX400; measured nuclide: ¹H; solvent: deuterated chloroform (containing TMS)). As a result, there was confirmed PHA constituted of a 3-hydroxy-5-(4-methylthio(phenoxy)valeric acid unit.

### Example 41

### Evaluation of Coating Property of Magnetic Capsule Construct

In order to confirm that the magnetic particles were completely protected and covered with the polymer, 0.1 g each of the obtained magnetic capsule constructs (30) to (42) was immersed for 2 hours in 100 ml of pure water heated to 70° C., and a metal content in the water was measured. As a result, the metal content was 3 ppm or less with all the capsule constructs. Based on these facts, it was deemed that metal ions did not elute.

### Example 43

α-fetoprotein (AFP) Immunoassay by Anti-AFP Antibody Carried on Magnetic Capsule Construct (18)

1. Immobilization of Antibody on Epoxylated PHA Magnetic Capsule Construct

The epoxylated magnetic capsule construct prepared in the example (18) was uniformly dispersed in a phosphate buffer (0.1 M; pH 7.4) (theoretical concentration 5 to 10×10⁸ particle/mL), and an anti-AFP antibody dissolved in a similar phosphate buffer was added in such a manner that the final ratio of the antibody to the magnetic capsule construct (18) became 5-10 μg/10⁷, and the mixture was mildly agitated by pipetting.

After a reaction for 1 hour at 30° C. by a rotary shaker, bovine serum albumin (BSA) for blocking was added so as to reach a final concentration of 0.3%, and a reaction was carried out for another 15 hours to obtain the antibody immobilized on the surface of the magnetic capsule construct (18).

2. Reaction with target component (antigen: AFP)

In a 5 mL Eppendorf tube (precoated with BSA), 500 μL of the dispersion of the anti-AFP-immobilized magnetic capsule construct (18), prepared in step 1, was mixed with 20 μL of an AFP solution of a concentration of 1 μg/mL and was reacted for 30 minutes at 37° C. The tube was brought into contact with a magnet to collect the magnetic capsule construct, and the supernatant was removed by decantation. Thereafter, 2 mL of a 0.04% NaCl solution was added and agitated in the tube. Then, the magnetic capsule construct was collected by the magnetic force and the supernatant was removed by decantation in the same manner as explained above. The similar rinsing operation was repeated three times.

3. Reaction with enzyme-labeled secondary antibody

An alkali phosphatase-labeled AFP antibody Fab′ was prepared according to J. Immunoassay, 4, 209 (1983) and Biochemistry, 11(12), 2291 (1972) and was mixed and dissolved in 0.1 M trishydrochloric acid buffer (containing 2% BSA, 1 mM MgCl₂ and 0.1 mM ZnCl₂; pH 7.5) and 500 μL of the mixture were added to the antigen AFP-binding capsule construct, prepared in step 2, and reacted for 10 minutes at 37° C. Then, the AFP-binding magnetic capsule construct reacted with the enzyme-labeled secondary antibody was rinsed in a similar manner as in step 2.

4. Detection

Into the tube containing the AFP-binding magnetic capsule construct reacted with the enzyme-labeled secondary antibody, 500 μL of glycine-NaOH buffer (0.1 M; pH 10.3; containing MgCl₂ (1 mM) and egg white albumin (250 mg/L) were added and, after a reaction for 5 minutes at 37° C., 500 μL of the afore-described buffer containing 4-nitrophenylphosphoric acid (final concentration 5.5 mM) were added and reacted for 60 minutes at 37° C. After the reaction, 500 μL of a 1 M NaOH aqueous solution were added to terminate the reaction, and absorbances in ultraviolet and visible regions were measured. As a result, there was detected absorbance at 405 nm, resulting from a reaction product by the labeled enzyme alkali phosphatase, thus confirming the recovery of the AFP.

### Example 44

Recovery of Concanavalin A Utilizing Cellopentaose-Immobilized Aminated Magnetic Capsule Construct (35)

1. Amination of Magnetic Capsule Construct (35)

The magnetic capsule construct (35) prepared in Example 33 was subjected to amination by adding 2,2′-(ethylenedioxy)-dimethylamine followed by a reaction for 15 hours at 30° C. After the reaction, it was rinsed five times with bis-2-methoxyethyl ether to eliminate remaining amine, and further rinsed with distilled water three times to obtain aminated magnetic capsule construct (35′).

2. Immobilization of Cellopentaose on Magnetic Capsule Construct (35′)

The magnetic capsule construct (35′) obtained in the step 1 was uniformly dispersed in a phosphate buffer as in Example 1 and was agitated, for 30 hours at 30° C., with a product of oxidation in advance of a non-reducing terminal of D-(+)-cellopentase (Sigma Co.) with sodium periodate to generate an aldehyde structure (—CHO: formyl group), thereby immobilizing cellopentaose on the magnetic capsule construct (35′). Thereafter, butanedienic anhydride was added and reacted to block excessive amino groups remaining on the surface of the magnetic capsule construct, and rinsing with distilled water was conducted three times to obtain a cellopentaose-immobilizing magnetic capsule construct (35′).

3. Recovery of Concanavalin A

Concanavalin A (Sigma Co.) and BSA were dissolved in the afore-described phosphate buffer, and the cellopentaose-immobilizing magnetic capsule construct (35′) obtained in the step 2 was added and reacted for 15 hours at 30° C., and the magnetic capsule construct was recovered by a magnetic force. The recovered particles were processed with sodium dodecylsulfate (SDS) and the elute was subjected to an SDS-PAGE analysis, which showed a substantially single band at 104 kDa, indicating the recovery of Concanavalin A.

### Example 45

Screening of DNA Fragment with Magnetic Capsule Construct (39)

1. Synthesis and amination of model nucleic acid M13p18ssDNA

A 20-mer oligonucleotide having a complimentary base sequence to a single-chain DNA of E. coli M13 phage mp18 and represented by a SEQ ID NO: 18 was synthesized by an auto DNA synthesizer (381A; ABI Inc.):

| | |
|---|---|
| SEQ ID NO: 18: | 5′-GTTGTAAAACGACGGCCAGT-3′ |

Then, an amino group (—NH₂) was introduced at the 5′ of the afore-described oligonucleotide 20-mer (compound “20”), utilizing a deoxyuridylate derivative monomer having an amino group (compound “19”) instead of an ordinary amidide reagent. Then, there were carried out, according to ordinary methods, a cleavage from the CPG support, a deprotection and a purification with a high speed performane liquid chromatography (HPLC).

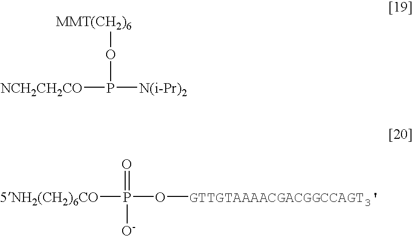

2. Immobilization of Aminated Probe Oligonucleotide on Magnetic Capsule Construct (39)

The carboxylated magnetic capsule construct (39) obtained in Example 36 was rinsed in advance with 0.01 M sodium hydroxide solution, and the obtained particles were reacted with EDC (1-ethyl-3-(3-diethylaminopropyl)-carbodiimide hydrochlorate) and aminated oligonucleotide of a theoretical amount of about double that of the carboxy group for 18 hours at 4° C., thereby obtaining a magnetic capsule construct carrying the probe oligonucleotide.

3. Synthesis and Labeling of Target Model Oligonucleotide

With respect to the oligonucleotide of the afore-described SEQ ID NO: 18, a completely complementary 20-mer oligonucleotide (SEQ ID NO: 19), an oligonucleotide with a mismatch in one base (SEQ ID NO: 20) and an oligonucleotide with a mismatch in two bases (SEQ ID NO: 21) were respectively synthesized by an automatic synthesizing equipment and purified by the ordinary method:

| | |
|---|---|
| SEQ ID NO: 19: | 5′-ACTGGCCGTCGTTTTACAAC-3′ |
| SEQ ID NO: 20: | 5′-ACTGGCCGTCCTTTTACAAC-3′ |
| SEQ ID NO: 21: | 5′-ACTGGCGGTCGTTATACAAC-3′. |

Each of the three purified model oligonucleotides was subjected to amination of the 5′ end, utilizing a deoxyuridylate derivative monomer (compound “19”) as in the step 1.

Separately, 170 mg of a cyanine dye (compound “21”) was dissolved in 5 ml of dry DMF, and 50 μl of dry pyridine was added according to a method disclosed in Japanese Patent No. 03368011. Then, 128 mg of DSC (disuccimidyl carbonate) was added, and agitation was conducted for 20 hours in a dark place at the room temperature. 150 ml of diethyl ether was added to the mixture, and the precipitate was collected, rinsed with diethyl ether and dried. The obtained active ester (compound “22”) was used for labeling the target model oligonucleotide (completely complementary strand: SEQ ID NO: 19).

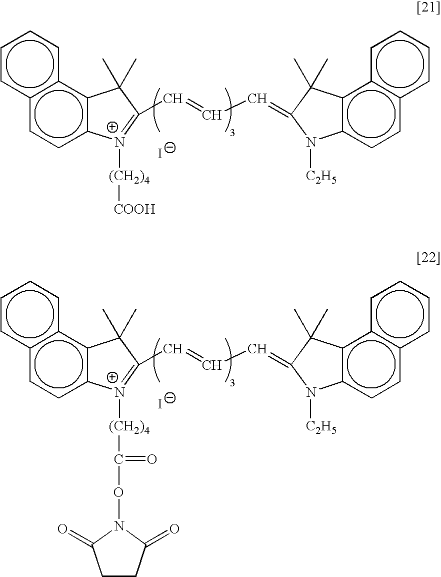

Also, compound [23], which was an active ester of an azulene dye, was used for labeling amino compounds of the afore-described oligonucleotides of mismatching in one base (SEQ ID NO: 20) and in two bases (SEQ ID NO: 21) (compound “24”).

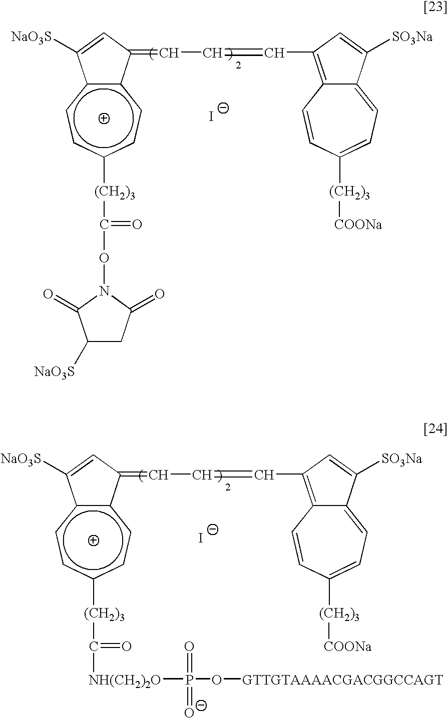

4. Screening of Completely Complementary Oligonucleotide

The magnetic capsule construct (39) having the probe oligonucleotide obtained in the step 2 and the labeled target oligonucleotide obtained in the step 3 were regulated so as to obtain a theoretical probe/target ratio of 1/10, then maintained for 2 minutes at 80° C. and returned to room temperature (hybridization condition).

The magnetic capsule construct was separated magnetically, and was subjected to a fluorescence measurement under an excitation with a laser of 780 nm. As a result, fluorescence was observed with a peak at about 820 nm, resulting from the compound of formula [21], thus confirming that the target DNA of SEQ ID NO: 15 was selectively recovered.

### Example 46

Recovery of Nonylphenol Utilizing Magnetic Capsule Construct (39) Carrying Anti-alkylphenol Antibody

1. Immobilization of Anti-Alkylphenol Antibody on Magnetic Capsule Construct (39)

A carboxy group on the PHA side chain of the carboxy-type magnetic capsule construct (39) prepared in the Example 36 was activated with N-ethyl-N′-(dimethylaminopropyl)carbodiimide (EDC) and N-hydroxysuccinimide (NHS) by the conventional method and reacted with twice the theoretical amount of an anti-alkylphenol antibody (Wako Pure Chemical Co.), thereby obtaining a magnetic capsule construct (39) carrying the anti-alkylphenol antibody.

2. Recovery and detection of nonylphenol

According to the method of “alkylphenol ELISA kit” (Wako Pure Chemical Co.), a standard liquid of nonylphenol and the magnetic capsule construct carrying the anti-alkylphenol antibody obtained in the step 1 were mixed and the reaction was accelerated by a switching operation of an electromagnet adhered to an external side of a container. After this operation was conducted for 30 minutes at 30° C., the electromagnet was maintained in an on condition to capture the magnetic capsule construct on the internal wall of the container, and the reaction liquid was removed. Then, a rinsing liquid was added to the container, and the rinsing was accelerated by a switching operation of the electromagnet. After repeating the rinsing step for 5 minutes with a new rinsing liquid three times, a solution of a chromophore-carrying substrate was added and an absorbance was measured. As a result, a strong absorption was observed at 450 nm, thus indicating that nonylphenol could be recovered and detected by this method.

### Example 47

Separation and Recovery of Liposome Utilizing Magnetic Capsule Construct (25) Carrying a Long-Chain Alkane

1. Immobilization of Dodecane on Magnetic Capsule Construct (25), Utilizing Dodecanethiol

1-dodecanethiol was dissolved in hexane. Then, sodium iodide, potassium carbonate and diethyl amine were added and mixed with the bromo-type magnetic capsule construct (25) prepared in Example 21 under agitation for 20 hours at room temperature to obtain a magnetic capsule construct carrying dodecane through a sulfide bond (—S—).

2. Recovery And Detection of Fluorescent Liposome

Liposome including FITC inside was prepared utilizing a “Liposome Kit” manufactured by Sigma Co. and according to a protocol thereof and was mixed under agitation with the dodecane-carrying magnetic capsule construct obtained in the step 1. After mixing for 15 minutes, a probe-shaped electromagnet was charged in a container and was turned on to capture the particles on the probe-shaped electromagnet. Then, the magnetic capsule construct was transferred to a container containing a rinsing liquid, and then released therein by turning off the electromagnet and was agitated for 15 minutes. After repeating this rinsing operation three times, a fluorescence measurement was conducted. As a result, a strong fluorescence was observed at 520 nm resulting from FITC, thus indicating that nonylphenol could be recovered and detected by this method.

### Example 48

### Separation and Recovery of Transfer Protein Utilizing Magnetic Capsule Construct (39) Carrying Consensus Binding Sequence Gene Fragment

Utilizing an affinity of a transcription factor ATF-2 having a basic leucine zipper and a consensus binding sequence thereof, separation and recovery of ATF-2 was carried out.

1. Synthesis Of Fragment Of Terminal Thiol-Type ATF-2 Consensus Binding Sequence

An automatic DNA synthesizer was employed to synthesize a single-stranded nucleic acid (TGACATCA, SEQ ID NO: 22). Also, at a 5′ terminus of the ssDNA of SEQ ID NO: 18, a sulfanyl group (—SH) was introduced (compound “25”) during the synthesis using the automatic DNA synthesizer with a Thio-Modifier (Glenn Research Inc.). Then, DNA was recovered by an ordinary deprotection, purified by a conventional method and used in the following experiments.

5′-HS—(CH₂)₆—O—PO₂—O-TGACATCA-3′ [25]

2. Immobilization of Terminal Thiol DNA Fragment on Magnetic Capsule Construct

A carboxy group on the PHA side chain of the carboxy-type magnetic capsule construct (39) prepared in Reference Example 8 was activated with N-ethyl-N′-(dimethylaminopropyl)carbodiimide (EDC) and N-hydroxysuccinimide (NHS) by an ordinary method, then activated to thiol by 2-(2-pyridinyldithio)ethanamine (PDEA) dissolved in a borate buffer (pH 8.5; regulated with NaOH) and subjected to an immobilization operation by adding the terminal thiol DNA fragment. After the reaction, the active group remaining on the magnetic capsule construct was deactivated with cysteine-NaCl.

3. Binding Reaction and Labeling

According to a protocol of Clontech Inc., the DNA fragment-carrying magnetic capsule construct obtained in the step 2 was introduced into a PBS solution of AFT-2, and the binding reaction based on the affinity of the DNA fragment and AFT-2 was accelerated by a magnet in a similar manner as in Example 46. After the reaction, rinsing was conducted in a similar manner as in Example 46, and an antigen-antibody reaction was conducted by a conventional method in a solution of anti-AFT-2 antibody (polyclonal) labeled with FITC.

4. Rinsing and Detection

After the antigen-antibody reaction, rinsing with a magnet and fluorescence detection were carried out in the same manner as in Example 46. As a result, there was confirmed a fluorescence having a maximum at 520 nm due to the label FITC, thus confirming recovery and detection based on the affinity binding of the transfer protein and the consensus sequence DNA fragment.

### INDUSTRIAL APPLICABILITY OF THE INVENTION

The construct of the invention, showing excellent dispersibility of a magnetic material and a magnetic response, low elution of metal ions to the exterior and excellent biological compatibility, can be widely applicable to various uses and fields.

Also, according to the invention, a capsule construct or a laminar construct, in which a magnetic material is coated, can be produced with an extremely simple process with a low environmental impact.

In particular, the present invention makes it possible to produce a construct, which coats a magnetic material with excellent dispersibility, without an oleophilic treatment on a metal or a metal oxide having magneticity, and a manufacture thereof.

Also, the capsule construct coating the magnetic material, obtained by the invention, can efficiently fix a target component-binding molecule by utilizing a side chain of a polyhydroxyalkanoate constituting the coating polymer, also shows an excellent dispersibility, and can achieve efficient separation, recovery and detection of a target component in a specimen under conditions close to those in vivo, because of the use of the high biological affinity polyhydroxyalkanoate as the polymer material coating the surface.

What is claimed is:

1. A method for separating a target component contained in a specimen, the method comprising:
a step of preparing a carrier in which a molecule having a binding affinity to the target component is immobilized on a surface;
a step of mixing the carrier and the specimen;
a step of binding the target component contained in the specimen mixed in the mixing step with the molecule immobilized on the carrier surface and having the binding affinity; and
a step of separating the target component together with the carrier from the specimen through the binding to the molecule having the binding affinity,
wherein the carrier includes a magnetic material and at least a part of the carrier is coated with a polyhydroxyalkanoate including at least one monomer unit selected from a group consisting of monomer units represented by chemical formulas [1] to [10] and [A] to [D]: 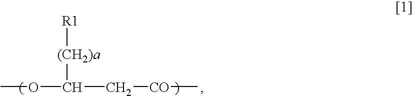
wherein a monomer unit is at least one selected from the group consisting of monomers having following combinations of R1 and a:
a monomer unit in which R1 is a hydrogen atom and a is any integer from 3 to 10;
a monomer unit in which R1 is a halogen atom and a is any integer from 1 to 10;
a monomer unit in which R1 is a chromophore and a is any integer from 1 to 10;
a monomer unit in which R1 is a carboxyl group or a salt thereof and a is any integer from 1 to 10; and
a monomer unit in which R1 is 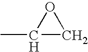
and a is any integer from 1 to 7; 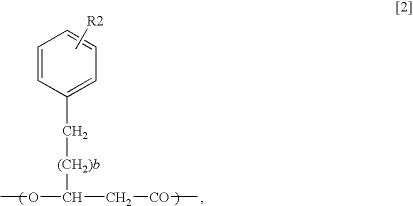
wherein b represents any integer from 0 to 7; R2 represents any one selected from the group consisting of a hydrogen atom, a halogen atom, —CN, —NO₂, —CF₃, —C₂F₅, —C₃F₇, a CH₃ group, a C₂H₅ group, a C₃H₇ group, a vinyl group, an epoxy group, and COOR21, where R21 representing an H atom, an Na atom or a K atom; and, if plural units are present, selections are made independently for each unit; 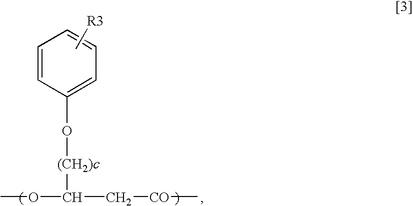
wherein c represents any integer from 1 to 8 and R3 represents any one selected from the group consisting of a hydrogen atom, a halogen atom, —CN, —NO₂, —CF₃, —C₂F₅, —C₃F₇, a CH₃ group, a C₂H₅ group, a C₃H₇ group, and a SCH₃ group; and, if plural units are present, selections are made independently for each unit; 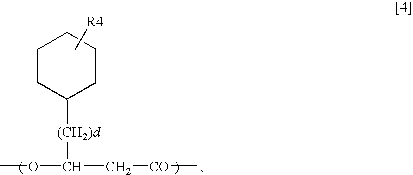
wherein d represents any integer from 0 to 8; R4 represents any one selected from the group consisting of an H atom, a CN group, an NO₂ group, a halogen atom, a CH₃ group, a C₂H₅ group, a C₃H₇ group, a CF₃ group, a C₂F₅ group, and a C₃F₇ group; and, if plural units are present, selections are made independently for each unit; 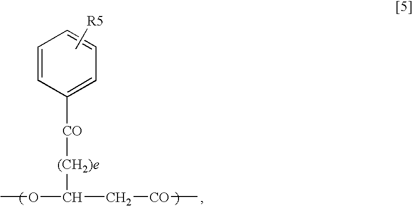
wherein e represents any integer from 1 to 8 and R5 represents any one selected from the group consisting of a hydrogen atom, a halogen atom, —CN, —NO₂, —CF₃, —C₂F₅, —C₃F₇, —CH₃, —C₂H₅, and —C₃H₇; 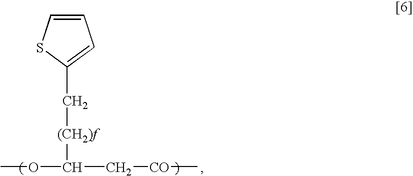
wherein f represents any integer from 0 to 7; 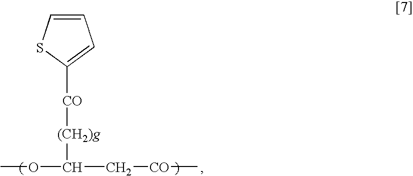
wherein g represents any integer from 1 to 8; 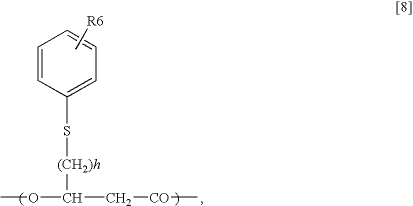
wherein h represents any integer from 1 to 7; R6 represents any one selected from the group consisting of a hydrogen atom, a halogen atom, —CN, —NO₂, —COOR′, —SO₂R″, —CH₃, —C₂H₅, —C₃H₇, —CH(CH₃)₂, and —C(CH₃)₃; R′ represents a hydrogen atom, Na, K, —CH₃, or —C₂H₅; and R″ represents —OH, —ONa, —OK, a halogen atom, —OCH₃, or —OC₂H₅; 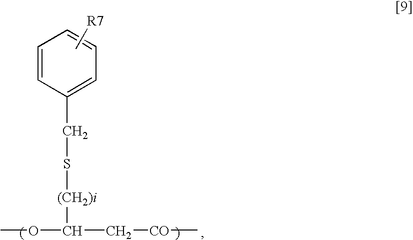
wherein i represents any integer from 1 to 7; R7 represents any one selected from the group consisting of a hydrogen atom, a halogen atom, —CN, —NO₂, —COOR′, and —SO₂R″; R′ represents a hydrogen atom, Na, K, —CH₃, or —C₂H₅; and R″ represents —OH, —ONa, —OK, a halogen atom, —OCH₃, or —OC₂H₅; 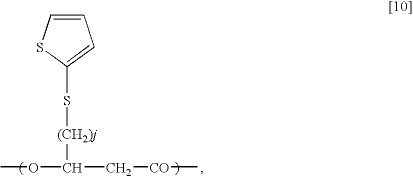
wherein j represents any integer from 1 to 9; 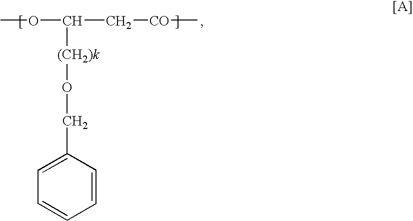
wherein k represents any integer from 1 to 8; 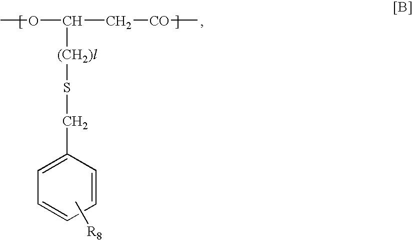
wherein 1 represents any integer from 1 to 8; R₈ represents any one selected from the group consisting of a CH₃ group, a C₂H₅ group, a C₃H₇ group, a (CH₃)₂—CH group, and a (CH₃)₃—C group; and, if plural units are present, selections are made independently for each unit; 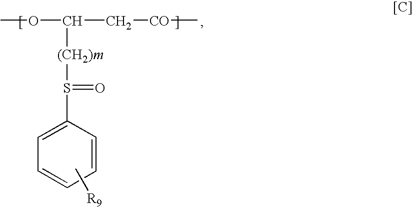
wherein m represents any integer from 1 to 8; R₉ represents an H atom, a halogen atom, a CN group, an NO₂ group, a CH₃ group, a C₂H₅ group, a C₃H₇ group, a (CH₃)₂—CH group, a (CH₃)₃—C group, COOR91, or SO₂R92, where R91 represents H, Na, K, CH₃, or C₂H₅, and where R92 represents OH, ONa, OK, a halogen atom, OCH₃, or OC₂H₅; and, if plural units are present, selections are made independently for each unit; and 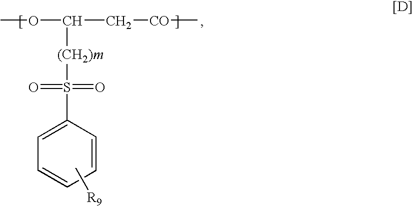
wherein m represents any integer from 1 to 8; R₉ represents an H atom, a halogen atom, a CN group, an NO₂ group, a CH₃ group, a C₂H₅ group, a C₃H₇ group, a CH₃)₂—CH group, a (CH₃)₃—C group, COOR91, or SO₂R92, where R91 represents H, Na, K, CH₃, or C₂H₅, and where R92 represents OH, ONa, OK, a halogen atom, OCH₃, or OC₂H₅; and, if plural units are present, selections are made independently for each unit.

2. The method according to claim 1, wherein the separating step comprises separating the carrier including the magnetic material from the specimen by applying a magnetic field to the carrier.

3. The method according to claim 1, wherein at least a part of the polyhydroxyalkanoate is a chemically modified polyhydroxyalkanoate.

4. The method according to claim 3, wherein the chemically modified polyhydroxyalkanoate is a polyhydroxyalkanoate having at least a graft chain.

5. The method according to claim 1, wherein at least a part of the polyhydroxyalkanoate is a crosslinked polyhydroxyalkanoate.

6. The method according to claim 1, wherein the magnetic material has a spherical shape, and a composition of the polyhydroxyalkanoate coating the magnetic material differs in a radial direction from the magnetic material.

7. The method according to claim 1, wherein the magnetic material comprises a metal or a magnetic metal compound.

8. The method according to claim 1, wherein a polyhydroxyalkanoate synthesizing enzyme is immobilized on the magnetic material.
